(19) United States
(12) Patent Application Publication
(10) Pub. No.: US 2016/0271072 A1
(43) Pub. Date: Sep. 22, 2016

(54) POLYMER NANOPARTICLES CONTAINING MULTIPLE AGENTS AND METHODS THEREOF

(71) Applicant: THE UNIVERSITY OF NORTH CAROLINA AT CHAPEL HILL, Chapel Hill, NC (US)

(72) Inventors: Leaf Huang, Durham, NC (US); Shutao Guo, Chapel Hill, NC (US); Lei Miao, Chapel Hill, NC (US)

(73) Assignee: The University of North Carolina at Chapel Hill, Chapel Hill, NC (US)

(21) Appl. No.: 15/031,052

(22) PCT Filed: Oct. 22, 2014
(86) PCT No.: PCT/US14/61794

§ 371 Date: Apr. 21, 2016

Related U.S. Application Data
(60) Provisional application No. 61/894,171, filed on Oct. 22, 2013.

(51) Int. Cl. A61K 9/51; A61K 31/7068; A61K 31/436; A61K 33/24

(52) U.S. Cl. CPC A61K 9/5153; A61K 33/24; A61K 31/7068; A61K 31/436; A61K 9/5123; A61K 9/5192

(57) ABSTRACT

Disclosed herein are polymer nanoparticles comprising i. a nano-precipitated bioactive compound, wherein the nano-precipitate is encapsulated by a lipid, and ii. a hydrophobic bioactive compound. Also provided herein are methods for preparing the nanoparticles and compositions comprising the nanoparticles and methods for the treatment of a disease or an unwanted condition in a subject comprising administering the polymer nanoparticles.

### CROSS-REFERENCE TO RELATED APPLICATIONS

This application is a national phase entry of International Application No. PCT/US2014/061794 filed Oct. 22, 2014, which claims priority to U.S. Provisional Application No. 61/894,171, filed Oct. 22, 2013, the entire contents of which are incorporated herein by reference.

### STATEMENT OF GOVERNMENT SUPPORT

This invention was made with government support under Grant No. CA149363 awarded by the National Institutes of Health. The government has certain rights in the invention.

### FIELD OF THE INVENTION

The present invention involves the delivery of a combination of bioactive compounds using lipid-comprising polymer nanoparticles.

### BACKGROUND OF THE INVENTION

Active agents, such as drugs, are often difficult to formulate into pharmaceutical formulations that have the desired properties, e.g., delivery of the active agent to a target and release profile of the active agent. While formulating a single active agent into an acceptable formulation is difficult, it is exponentially more difficult to formulate two or more active agents in the same formulation. Though it is desirable to formulate multiple active agents into a single formulation because multi-drug therapy is effective against diseases such as cancer, it is often not possible to overcome the difficulties associated with preparing these formulations.

Polymer nanoparticles are known as active agent delivery vehicles. However, loading of the active agent in the nanoparticle is very difficult and can suffer from small loading efficiency. Existing nanoformulations suffer from low loading efficiency and burst drug release kinetics particularly when used with a drug having low solubility. The co-loading of two active agents is even more difficult. In prior methods, the two active agents are required to have similar physiochemical properties.

Since useful active agents can have different physiochemical properties, it would be beneficial to be able to co-load any active agents into a single nanoparticle, particularly active agents having physiochemical properties that are different from each other. The present disclosure addresses these needs.

### BRIEF SUMMARY OF THE INVENTION

Provided herein are compositions that include nanoparticles comprising, i. at least one nano-precipitated bioactive compound, wherein the precipitate core is encapsulated by a lipid or has at least a portion of its surface coated with a lipid; and ii. a hydrophobic bioactive compound that is different from the nano-precipitated bioactive compound. Because the nanoparticles contain nano-precipitates of bioactive compounds, the nanoparticles are capable of formulating essentially insoluble forms of bioactive compounds. The nanoparticles can also contain a hydrophobic bioactive compound. Also provided herein are methods for the treatment of a disease or an unwanted condition in a subject, wherein the methods comprise administering the nanoparticles. The nanoparticles can comprise any type of nano-precipitated bioactive compound, including but not limited to, polynucleotides, polypeptides, and drugs.

The nanoparticles can be used to deliver bioactive compounds to cells. Therefore, provided herein are methods for delivering two or more bioactive compounds to a cell, wherein the method comprises contacting a cell with a nanoparticle comprising at least one nano-precipitated bioactive compound and at least one hydrophobic bioactive compound.

Nanoparticles can comprise a targeting ligand and are referred to as targeted nanoparticles. These targeted nanoparticles can specifically target the bioactive compound to diseased cells, enhancing the effectiveness and minimizing any possible toxicity of the nanoparticles.

Further provided herein are methods for making the nanoparticles.

These and other aspects of the invention are disclosed in more detail in the description of the invention given below.

### BRIEF DESCRIPTION OF THE FIGURES

FIG. 1 depicts transmission electron microscopy (TEM) images: MBA-PEG-PLGA NPs loaded with 1.9 wt % of DOPA-CDDP core (A), 4.5 wt % of DOPA-CDDP core (B) and 4.5 wt % of DOPA-CDDP core with 2.2 wt % of RAPA (C). The NPs were negatively stained with uranyl acetate.

FIG. 2 depicts the LE (loading efficiency) and EE (encapsulation efficiency) (A), size and polydispersity measured by DLS (B) of PLGA NPs loaded with DOPA-CDDP core; characterization of LE and EE (C), size and polydispersity measured by DLS (D) of PLGA NPs loaded with RAPA. The number of DOPA-CDDP core per MBA-PEG-PLGA NPs (E and F) can be controlled by altering its feeding ratio and is not affected by the presence of RAPA.

FIG. 3 depicts in vitro release kinetics of CDDP and RAPA in PBS at 37° C. from NPs (A). The loading of CDDP and RAPA in (CDDP+RAPA) NPs was 4.5 wt % and 2.2 wt %, respectively. Cellular uptake of MBA-PEG-PLGA NPs determined using ICP-MS (B). The cells were incubated with NPs for 4 h. Cell toxicity of RAPA, CDDP and their combination in A375-luc cells (C); Cell toxicity of RAPA NPs, CDDP NPs and (CDDP+RAPA) NPs in A375-luc cells (D). The apoptosis of A375-luc cells induced by incubation with drugs for 24 h. The number of apoptotic cells was counted by flow cytometry (E).

FIG. 4 shows the apoptosis of A375-luc cells induced by incubation with drugs for 24 h. Cells were stained with Annexin V-FITC/PI and imaged using fluorescent microscopy.

FIG. 5 depicts the effects of empty NPs, RAPA NPs, CDDP NPs and (RAPA+CDDP) NPs on tumor growth (A) and body weight (B) respectively of A375-luc tumor bearing mice. The arrowheads indicate the time of injection. RAPA was dosed intravenously at a dose of 0.15 mg/kg; CDDP was dosed intravenously at a dose of 0.30 mg/kg. The results are displayed as mean±SEM (error bars) of four animals per group.

FIG. 6 shows tumor sections stained with Masson Trichrome, and the blue color captured collagen content. The collagen content was quantified using ImageJ.

FIG. 7 depicts the RAPA and CDDP combination remodeled the tumor microenvironment. (RAPA+CDDP) NP depleted the stroma and showed anti-angiogenesis and blood vessel normalization in tumor.

FIG. 8 depicts the anti-angiogenesis effect of drug on A375-luc xenograft tumor was investigated by CD-31 staining; cancer associated fibroblasts were stained by α-SMA antibody; apoptosis of cells in A375-luc tumor was indicated by TUNEL assay (A). The number denotes the average number of CD-31 positive vessels per microscopic field; the percentage denotes the average percentage of α-SMA±fibroblasts and the percentage of TUNEL positive cells, respectively. Vessel area, quantified using ImageJ, was normalized to PBS control (B).

FIG. 9 depicts anti-angiogenesis effect of drug on A375M xenograft tumor that was investigated by CD-31 staining; apoptosis of cells in A375M tumor was indicated by TUNEL assay (C). The number denotes the average percent of CD-31 positive area per microscopic field and the percentage of TUNEL positive cells, respectively.

FIG. 10 depicts a preferred formulation of MBA-PEG-PLGA NPs loaded with CDDP Cores and GMP Cores. a. TEM image of DOPA-GMP cores; b. TEM image of DOPA-CDDP cores; TEM image of MBA-PEG-PLGA NPs loaded with 5 wt % of DOPA-GMP core and 1 wt % of DOPA-CDDP core. LE: Loading efficiency; EE: encapsulation efficiency.

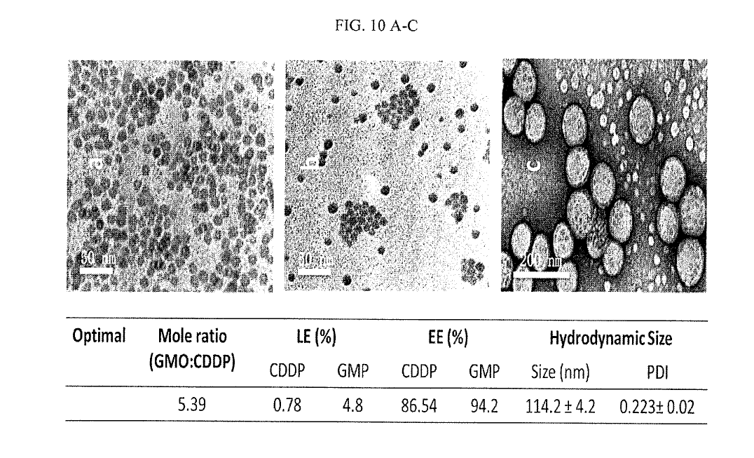

FIG. 11 shows characterization of LE and EE of MBA-PEG-PLGA NPs loaded with DOPA-CDDP core and DOPA-GMP core. Molar ratio between GMP and CDDP in MBA-PEG-PLGA NPs was initially controlled to 5:1, the feed ratio (expected total LE, green bars) of the two drugs were changed from lwt % to 8 wt % accordingly. Actual ratio (purple triangles) between GMP and CDDP in MBA-PEG-PLGA NPs were calculated (A); LE and EE of CDDP and GMP in the aforementioned formulations were studied (B); particle size and polydispersity were measured by DLS (C). Then total feed ratio (Expected ratio) were maintained at 6 wt %, molar ratio between GMP and CDDP were changed from 0.5:1, 1:1, 3:1 to 5:1. Actual ratio (purple triangles) between GMP and CDDP in MBA-PEG-PLGA NPs were calculated (D); LE and EE of CDDP and GMP in the aforementioned formulations were studied (E); particle size and polydispersity were measured by DLS (F). LE: Loading efficiency; EE: encapsulation efficiency.

FIG. 12 depicts the in vitro release kinetics of CDDP and GMP in PBS at 37° C. from NPs. The loading of CDDP and GMP in (CDDP+GMP) NPs was 0.88 wt % and 5 wt %, respectively.

FIG. 13 depicts a cytotoxicity study of free GMP, CDDP and free drug combination at variable molar ratios (A) and the corresponding CI vs Fa plot (C); Cytotoxicity study of free GMP, CDDP and free drug combination at molar ratio 5:1; single MBA-PEG-PLGA NPs loaded with DOPA-GMP core and DOPA-CDDP core separately, MBA-PEG-PLGA NPs co-loaded with DOPA-GMP core and DOPA-CDDP core at molar ratio 5:1 (B) and the corresponding CI vs Fa plot of PLGA combo (D). Note that the total drug concentrations, IC 50 of each treatment are presented, n=5.

FIG. 14 depicts the effects of MBA-PEG-PLGA NPs loaded with single cores or co-loaded with DOPA-GMP and DOPA-CDDP core on tumor growth of bladder tumor bearing mice. The arrows indicate the time of injection. GMP was dosed intravenously at 8 mg/kg and CDDP at 1.6 mg/kg (in both free drug and nanoparticle formulation). The results are displayed as mean±SD of 4-5 animals per group.

FIG. 15 depicts a polymer nanoparticle comprising diverse bioactive compounds that can be prepared by the methods disclosed herein.

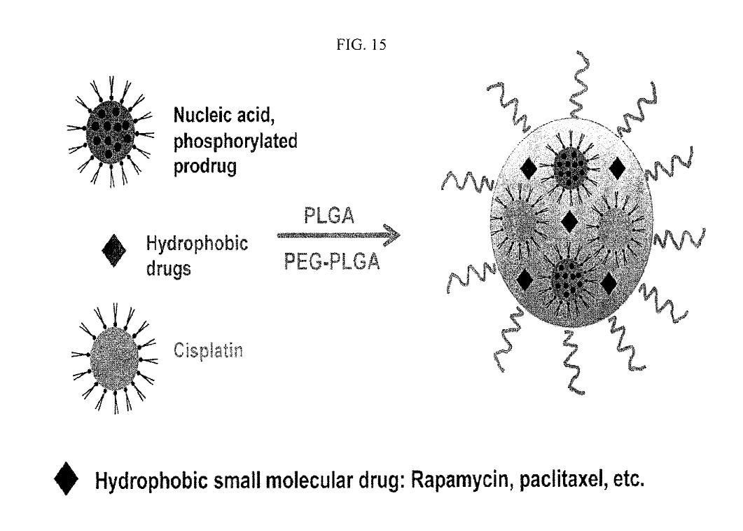

FIG. 16 depicts the cumulative release of CDDP and GMP in PLGA combo and PLGA NPs.

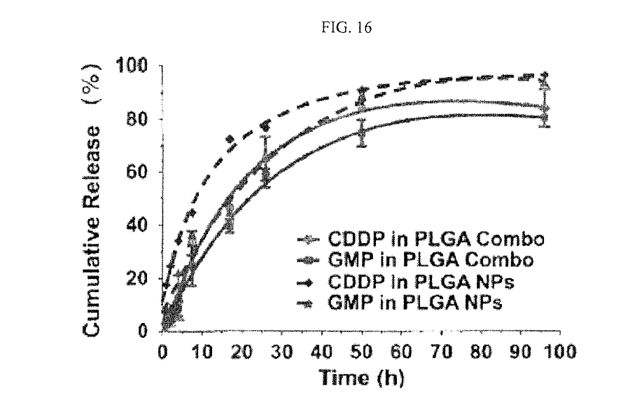

FIG. 17 depicts cell toxicity of MBA-PEG-PLGA NPs in A375-luc cells.

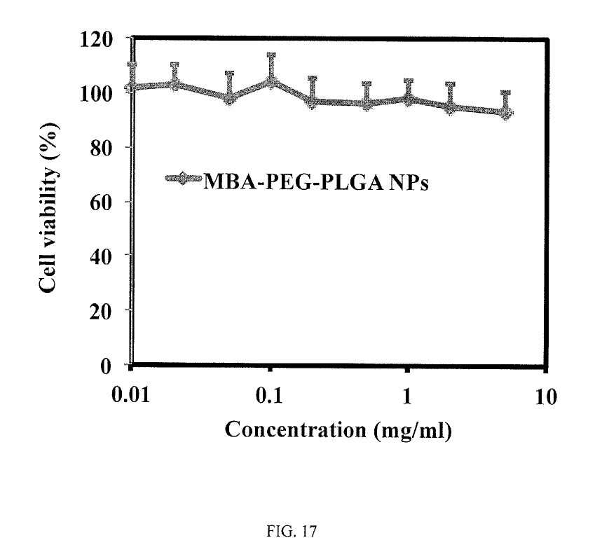

FIG. 18 shows apoptosis of cells induced by free RAPA, CDDP and (RAPA+CDDP). The concentration of RAPA was 0.36 μM and the concentration of CDDP was 2.0 μM. The molar ratio of CDDP to RAPA was 5.5.

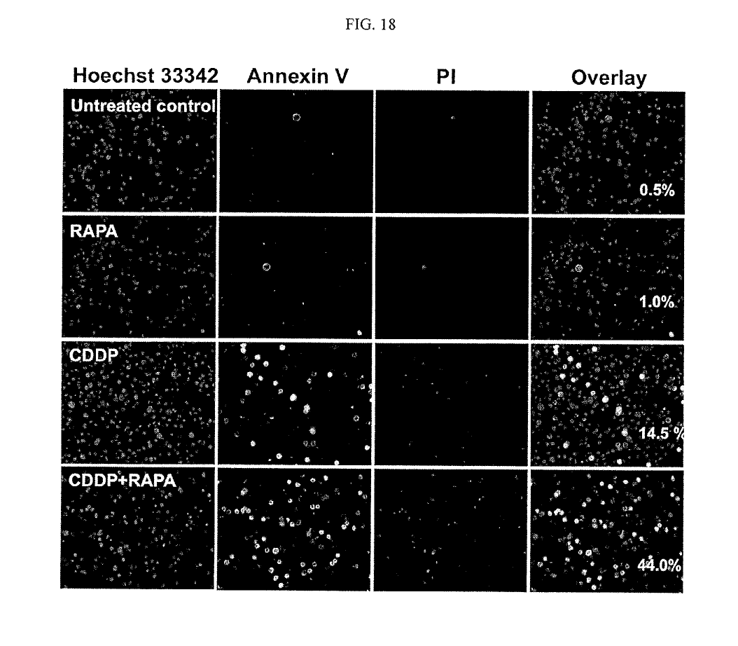

FIG. 19 shows apoptosis of cells induced by RAPA NPs, CDDP NPs and (CDDP+RAPA) NPs. The concentration of RAPA was 0.36 μM and the concentration of CDDP was 2.0 μM. The molar ratio of CDDP to RAPA was 5.5.

FIG. 20 depicts H&E staining of kidney tissues from mice treated with PBS, empty NP, RAPA NP, CDDP NP, and (CDDP+RAPA) NP.

FIG. 21 (A) depicts fabrication of PLGA-PEG-Anisamide NP (PLGA NP) containing CP cores and GMP cores via a single step solvent displacement method. (B) Cisplatin and GMP, which are ratiometrically encapsulated in PLGA NP, are ratiometrically delivered into the tumor and exhibit strong synergistic anti-tumor efficacy.

FIG. 22 depicts (A) dual-drug ratiometric loading in Combo NP. EE and DL of GMP and cisplatin in Combo NP while the total loading of drugs was fixed at 6 wt %; (B) EE and DL of GMP and cisplatin in Combo NP while the feed molar ratio of GMP to cisplatin was fixed at 5:1; (C) TEM image of 5.5 wt % total drug loading of Combo NP with molar ratio of GMP and cisplatin of 5.3:1; (D) EDS spectra of Combo NP; (E) Both platinum from CP cores and fluorine from GMP cores were observed in a single NP indicating actual loading of dual drugs in single NP. XPS spectrum of Combo NP. Molar ratio of GMP and cisplatin was also quantified using atomic ratio of fluorine and platinum. Spectrum of Pt 4F and spectrum of F 1S, from which, area of peaks are integrated for atom quantification.

FIG. 23 depicts (A) ratiometric cellular uptake and release of dual drugs from Combo NP. Uptake of cisplatin and GMP in Combo NP, Sepa NP, and free drugs at 37° C. for 4 h in UMUC3 cells; (B) Accumulative uptake of Combo NP loaded with cisplatin and GMP in UMUC3 Cells; (C) In vitro release kinetics of cisplatin and GMP from Combo NP and single NP in PBS at 37° C.; and (D) intracellular release of cisplatin and GMP from Combo NP; (E) IC₅₀ of free GMP, cisplatin, and Combo free at molar ratio 5.3:1, as well as single drug NP and Combo NP at molar ratio 5.3:1; (F) X-axis indicated the total concentration of dual drugs or single drug formulations. The corresponding CI vs Fa plots of Combo NP and Combo free were shown. DL of cisplatin and GMP in Combo NP is 0.8 wt % and 4.6 wt % respectively, while DL of cisplatin and GMP in single NP is 4.4 wt % and 4.2 wt % respectively. n.s.: no significant difference; * P<0.05; ** P<0.01.

FIG. 24 depicts (A) Tumor inhibition effects of free drugs, Combo free, cisplatin NP, GMP NP, Sepa NP and Combo NP on a stroma-rich UMUC3 bladder cancer xenograft model; (B) Arrows in panel A indicate time of injection. The tumors were treated with three IV injections at a dose of 1.9 mg/kg cisplatin and 12 mg/kg GMP in all the treatment groups. Tumor accumulation of cisplatin and GMP was calculated 10 h post injection of Combo NP, Sepa NP and Combo free at the injection dose of 1.9 mg/kg cisplatin and 12 mg/kg GMP into nude mice bearing stroma-rich bladder cancer xenograft tumors; (C) Anti-tumor effects of multiple low dosing schedule and single high dosing schedule were compared, N=5; * P<0.05; ** P<0.01; ^{#}P>0.2; ^{##}P>0.5; n.s: non-significant difference. ID/g: injected dose per gram tissue (tumor); (D) Dosing schedule and tumor images of single high dose and multiple low doses of Combo NP.

FIG. 25 depicts Apoptosis (A) and proliferation (B) of tumor cells in vivo after administration of different treatments. Expression of XPA and ERCC-1, common in nucleotide excision repair (NER) systems, after three dosage systemic treatments (C). The formation of Pt-DNA adduct (green) in tumor cells detected by anti-Pt-DNA adduct antibody after systemic treatment (D). Bar chart in D is a quantitative analysis of % of Pt-DNA adduct in tissue sections. Five randomly selected microscopic fields were quantitatively analyzed on Image J. * P<0.05; ** P<0.01.

FIG. 26 depicts TEM image of GMP cores (a); TEM image of CP cores (b). GMP and CP cores have similar size and morphology.

FIG. 27 depicts Western blot analysis of Sigma Receptor, an epithelial cell surface marker, in non-small cell lung cancer H460 and bladder cancer cell line UMUC3. Results showed that UMUC3 has an expression of sigma receptor comparable to H460, indicating the feasibility of anisamide targeting effect.

FIG. 28 depicts EE of GMP in GMP NP and cisplatin in cisplatin NP while changing the feed loading of single drug cores in PLGA NP.

FIG. 29 depicts size and PDI of Combo NP with total feed loading fixed at 6 wt % (a) or feed molar ratio of GMP/cisplatin fixed at 5:1 (b).

FIG. 30 depicts Size and PDI of 4.4 wt % cisplatin NP, 4.2 wt % GMP NP and 5.5 wt % Combo NP with molar ratio of GMP and cisplatin 5.3:1 were measured by DLS.

FIG. 31 depicts uptake of total drug from Combo NP modified with or without anisamide at 37° C. for 4 h on UMUC3 cells. Haloperidol acts as an inhibitor of sigma receptor.

FIG. 32 In vitro release kinetics of cisplatin and GMP from Combo NP and single NP in pH 5.6 PBS at 37° C. (n.s.: no significant difference).

FIG. 33 depicts effect of different treatments on UMUC3 tumor weight after three dosages. Arrows indicate the day of injection.

FIG. 34 depicts Western blot of PARP, cleaved PARP, caspase-3 and GAPDH in the tumor lysates after three dose treatment.

FIG. 35 depicts biodistribution of Combo NP, Sepa NP, and Combo free in major organs 10 h post intravenous injection into UMUC3 stroma-rich tumor bearing nude mice. (% ID/g tissue: percentage of injected dose per gram tissue).

FIG. 36 depicts HE staining of major drug accumulating organs after three injections of treatments.

FIG. 37 depicts Hematological test of whole blood collected from healthy nude mice treated with three doses of free drugs and NPs as indicated (n=4). WBC, white blood cell; HCT, hematocrit; PLT, platelet; HGB, hemoglobin; RBC, red blood cell. (If not indicated, no significant difference between PBS group and the treatment group. * P<0.05; ** P<0.01)

### DETAILED DESCRIPTION OF THE INVENTION

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims.

Polymer nanoparticles (NPs) have been prepared that contain multiple bioactive compounds. At least one of the bioactive compounds is in the form of a nano-precipitate, e.g. as described in US Appl. Pub. No. 2012/0201872 and PCT/US2013/061985, each of which is herein incorporated in its entirety. In embodiments, the nano-precipitated core is coated with a single lipid layer. In embodiments, at least one of the bioactive compounds is a hydrophobic bioactive compound. In embodiments, the nanoparticles can comprise additional nano-precipitate cores containing a different bioactive compound. As described fully herein, the bioactive compounds are co-loaded and encapsulated in a polymer nanoparticle.

In particular, a MBA-PEG-PLGA NP encapsulating DOPA-cisplatin (CDDP) cores and rapamycin (RAPA) has been prepared. The methods described herein encapsulate CDDP directly into MBA-PEG-PLGA NP with efficient loading and encapsulation. Unexpectedly, when co-encapsulated with RAPA, DOPA-CDDP cores improved RAPA loading by about 3.48-fold. Additionally, a controlled release profile was demonstrated for both CDDP and RAPA.

Advantageously, cytotoxicity of the combined drugs was enhanced by MBA-PEG-PLGA NP delivery. Combined RAPA and CDDP reduced the number of cancer-associated fibroblasts and the expression of collagen within xenograft tumors, had an anti-angiogenesis effect and normalized tumor blood vessels. Thus, it has been shown that bioactive compounds having different physiochemical properties can be efficiently co-loaded into polymer nanoparticles to treat a variety of diseases with the advantage of synergistic activity between components.

Combined therapy against both tumor and supporting microenvironment can potentially be more effective due to synergistic enhancement of drug action. As described herein, difficulties in co-encapsulation of drugs with diverse physical properties into the same nanoparticulate formulation has been overcome. By way of a non-limiting example, co-encapsulation of hydrophilic, inorganic cisplatin with Rapamycin into PLGA using a nanoprecipitation method is described herein. DOPA-coated cisplatin cores with a hydrophobic surface were engineered. Encapsulating the DOPA-coated cisplatin into PLGA NP was unexpectedly enhanced by the presence of Rapamycin and by modulating the compatibility between Rapamycin and PLGA. These NP induced apoptosis in A375 melanoma tumor cells, exhibited anti-angiogenic effects, and modulated the tumor microenvironment through down regulation of collagen and depletion of cancer associated fibroblasts. Other bioactive compounds can be used as well. Described herein is the co-encapsulation of multiple bioactive compounds having varied physicochemical properties, e.g., gemcitabine cores and cisplatin cores along with hydrophobic Rapamycin.

Tumor microenvironment plays an important role in angiogenesis, tumor progression, invasion and metastasis. Remodeling the tumor microenvironment may be a powerful strategy to sensitize tumor cells to chemotherapy. Therefore, as set forth herein, a potential therapeutic strategy can be based in part on treatment-induced change in the tumor microenvironment. Rapamycin (RAPA), an mTOR inhibitor with anti-angiogenic and anti-tumor activity, can sensitize A375 melanoma cells to cisplatin (CDDP). Combination therapy using both CDDP and RAPA at an optimized molar ratio can promote synergy between the two drugs. However, previous attempts to encapsulate these drugs into poly (lactic-co-glycolic acid) (PLGA) nanoparticles (NPs) were not efficient due to incompatibility between free drug and the polymer matrix. A single drug can be stabilized as previously described in US Appl. Pub. No. 2012/0201872 and PCT/US2013/061985, e.g., by utilizing a hydrophobic nano-precipitation of CDDP stabilized by dioleoylphosphatidic acid (DOPA). Disclosed herein is a targeted polymer, e.g. PLGA, nanoparticle comprising efficient loading with both hydrophobic DOPA-CDDP cores and RAPA at a molar ratio that promotes synergistic anti-tumor activity between CDDP and RAPA.

The PLGA NPs demonstrated the ability to promote changes in the microenvironment supporting A375-luc melanoma xenograft tumors after treatment. Importantly, PLGA NP demonstrated controlled release of both drugs, induced apoptosis in cultured A375-luc melanoma cells, and significantly modulated the vasculature and microenvironment of tumors in an A375-luc xenograft model. As disclosed herein, this method can provide co-loading of other lipid-coated nano cores containing drug or imaging agent to be combined with hydrophobic drugs to offer new treatment options.

Provided herein are nanoparticles as depicted in schemes 1 and 3 below comprising two or more nano-precipitated bioactive compounds, or at least one nano-precipitated bioactive compound and an additional hydrophobic bioactive compound, wherein the nano-precipitated compound is encapsulated or coated on at least a portion thereof by a lipid. In general, a nano-precipitate of a bioactive compound can be prepared by mixing two reverse micro-emulsions containing reactants. In this way, a nano-precipitate is formed and coated with a single lipid layer. Lipid coated cores are described herein and in US Appl. Pub. No. 2012/0201872 and PCT/US2013/061985. Utilizing the methods described herein for nanoparticle formation, bioactive compounds that are difficult to formulate can not only be formulated but advantageously can be formulated along with other bioactive compounds having different physiochemical properties. By converting otherwise non-candidate compounds into potential bioactive compounds and successfully combining them with other agents in a single polymer nanoparticle formulated for delivery, the subject matter described herein makes a significant contribution to medicine, in particular, cancer chemotherapy.

As used herein the term “nano-precipitate” refers to a nano-precipitated bioactive compound or precursor thereof. In embodiments, the bioactive compound has low-solubility in water and oil or is essentially insoluble in water and oil, and a lipid encapsulating or coating at least a portion of the surface of the bioactive compound. The term “low-solubility” means that the nano-precipitated bioactive compound or precursor thereof is not solubilized in water and oil to an appreciable amount.

As used herein, the bioactive compound is prepared as a nano-precipitate by contacting the compound or a precursor of the compound with a species that forms a nano-precipitate of the bioactive compound. As used herein, the nano-precipitated bioactive compound has a lipid coating as described elsewhere herein. Thus, a nano-precipitate is distinguishable from bulk precipitates. Additionally, bulk precipitates do not have nano-sized lipid coated particles. Utilizing the methods disclosed herein, bioactive compounds can be prepared as nano-precipitates and formulated in nanoparticles that contain at least one other different bioactive compound.

In this embodiment, the nano-precipitated bioactive compound is formed as a salt in a reverse microemulsion that results in the nano-precipitated bioactive compound having at least a portion of its surface coated by a lipid. In embodiments, the nano-precipitate consists essentially of the bioactive compound in its nano-precipitated salt form and a lipid coating. Preferably, the nano-precipitate consists of the bioactive compound in its nano-precipitated salt form and a lipid coating. In some cases, more than one bioactive compound can be co-precipitated by a single ion to form mixed insoluble salts that are nano-precipitates. For example, both etoposide phosphate and gemcitabine phosphate can be nano-precipitated using InC13 in the methods described herein. Polymer nanoparticles containing nano-precipitates of mixed Indium salts of etoposide phosphate and gemcitabine phosphate can therefore be prepared. Different bioactive compounds in the polymer nanoparticles can inhibit the same or different biochemical pathways in the target cells to perform additive or synergistic therapeutic activities.

In some instances, a bioactive compound can be higly potent, however, its practical applicablity is severely limited by the high toxcity, low bioavailability, instability or the like. Accordingly, some embodiments are directed to polymer nanoparticles comprising an encapsulated, nano-precipitated bioactive compound, wherein the bioactive compound is highly soluble yet possesses above-mentioned undesirable properties. In some embodiments, such highly soluble bioactive compounds can be precipitated out of a solution using appropriate metal counter ions. Such metal ions include, but not limited to In⁺³, Gd⁺³, Mg⁺², Zn⁺² and Ba⁺².

Importantly, the lack of required seeding material in the nano-precipitate provides for substantially increased loading potential. Loading of the nanoparticle with the nano-precipitate can result in an amount of nano-precipitate of at least 10% wt of said nanoparticle. Preferably, the amount is from about 20 to about 70% wt or from about 20% to about 85% wt; from about 30 to about 60%; and more preferably from about 40% wt to about 50% wt. The nanoparticle can further comprise components that are specifically listed elsewhere herein.

As used herein, the term “ratiometric” refers to the deliberate, controlled loading of an amount of one bioactive compound relative to the amount of one or more other biocative compounds present in the nanoparticles. The term “controlled” refers to a process that is capable of precise loading and is distinguished from methods that cannot exert control on the precise amount of loading. Ratiometric amounts of a first bioactive compound relative to a second bioactive compound are from about 1:1,000 to about 1,000 to 1. Preferably, the ratiometric amounts of bioactive compounds provide a synergystic effect as disclosed elsehwere herein. Advantageously, the co-loading can be performed ratiometrically at desired levels with excellent precision. For example, a ratio can be 1:1, wherein each drug is present at the same ratio either by molar or by weight. A preferred molar ratio is from about 2:1; 3:1; 4:1; 6:1; 7:1; or most preferably about 5:1, DOPA-GMP core:DOPA-CDDP core.

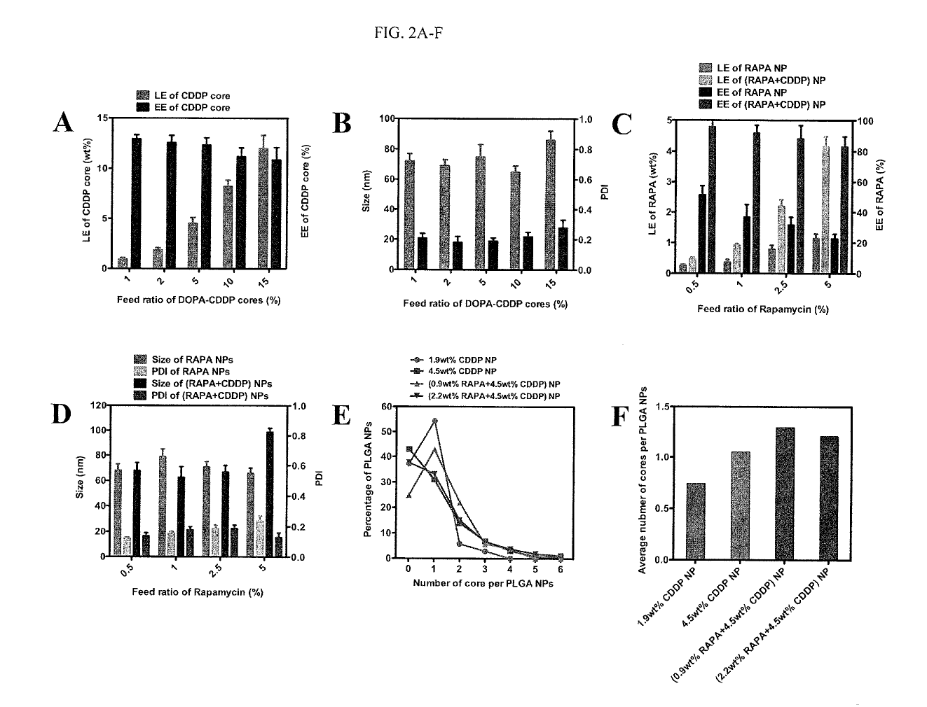

The number of DOPA-CDDP cores per NP can be controlled by adjusting the DOPA-CDDP core: MBA-PEG-PLGA input ratio. Rapamycin had no effect on encapsulation or NP morphology (FIG. 2F).

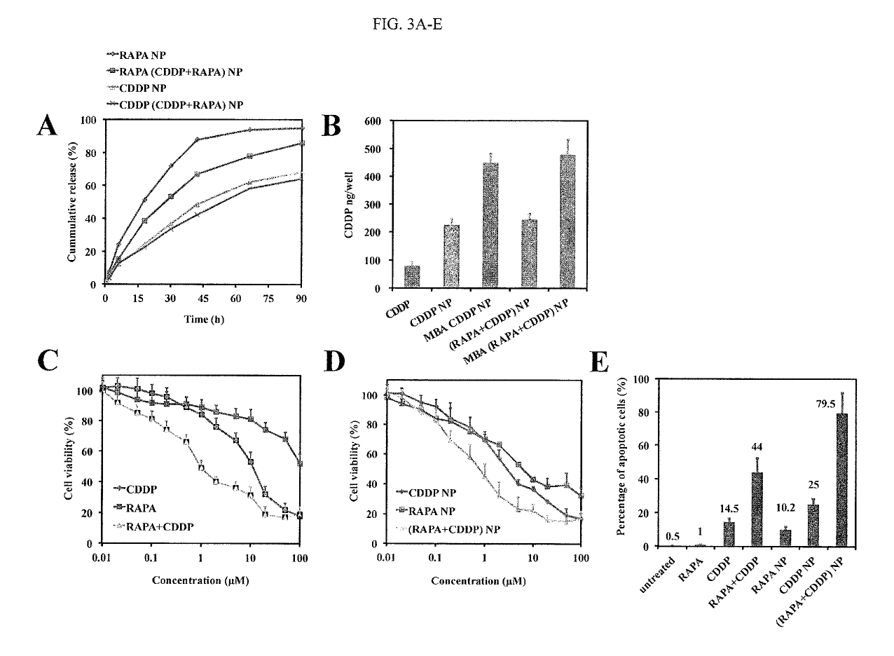

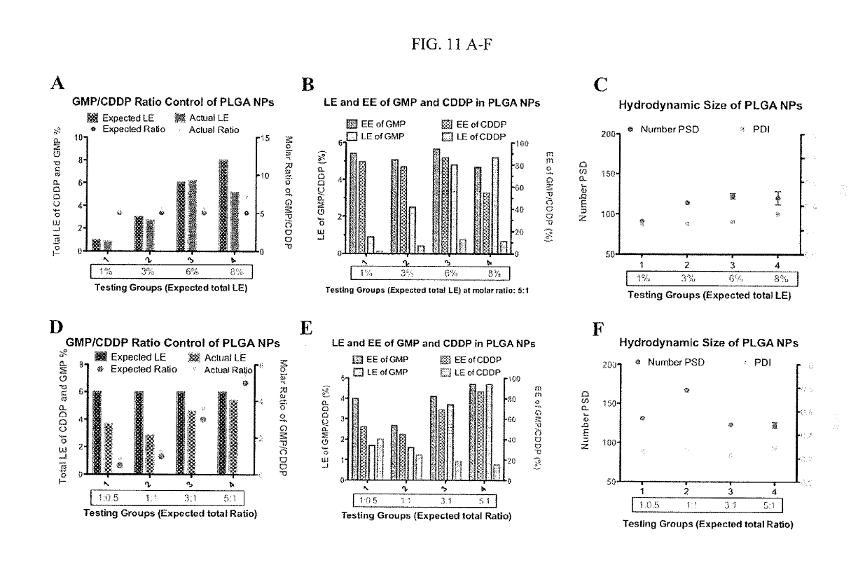

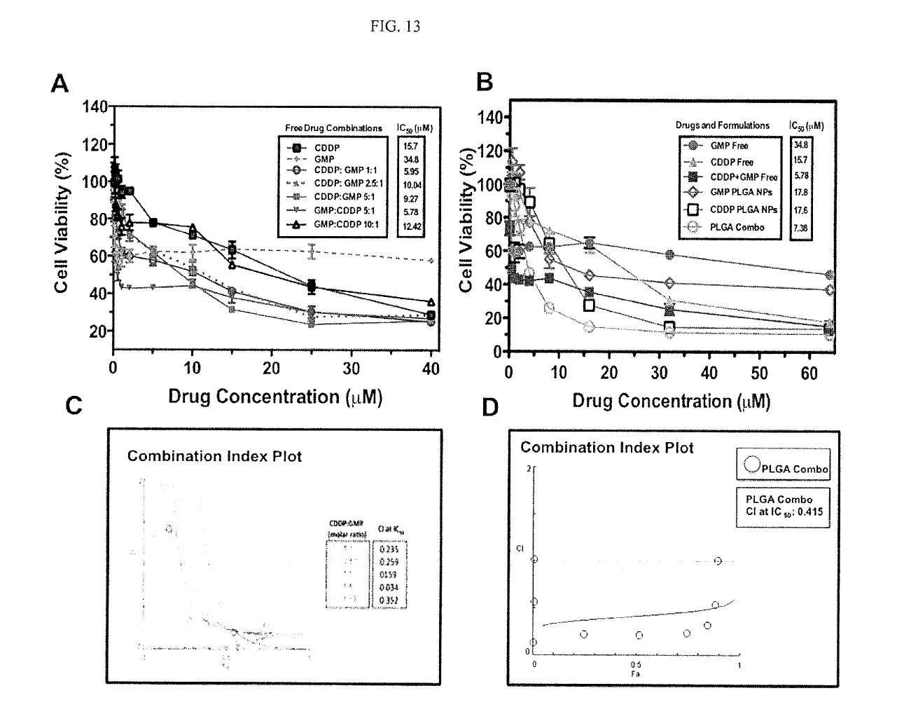

Dual-loaded (DOPA-GMP and DOPA-CDDP) MBA-PEG-PLGA NPs with ratiometric loading were prepared by adjusting the input molar ratio of CDDP and GMP. The calculated ratio of GMP to CDDP in PLGA NPs was nearly the same as the input ratio when input GMP:CDDP was set at 5:1 and the total loading was below 6 wt %. Additionally, the encapsulation efficiency for the two drugs was similar. The ratio of GMP to CDDP in MBA-PEG-PLGA NPs was well controlled when the total input of two drugs to PLGA was set at 6 wt %. This resulted in an encapsulation efficiency as high as 90% (FIGS. 11 D and E). TEM images show that the MBA-PEG-PLGA NPs were spherical and approximately 80-100 nm in diameter. Cores were clearly observed in each particle. These results confirm that the ratio of two drugs with drastically different properties can be controlled by formulating them in this manner Data also show that free GMP and CDDP exhibit synergistic effects on proliferation of cultured UMUC-3 bladder cancer cells withmaximal effect at a ratio of 5:1, which is also the accepted ratio used in clinical treatment (FIG. 13C). Moreover, for effective combination therapy, the formulation must achieve effective release rate for both drugs. Measurement of CDDP and RAPA release by ICP-MS and HPLC (FIG. 3) showed similar, sustained rates for both drugs. Neither drug affected the release rate of the other.

Nanoparticles, through both passive and active targeting, can enhance the intracellular concentration of drugs in cancer cells while avoiding toxicity in normal cells. Surface PEGylated nanoparticles can efficiently deliver nucleic acid, chemo-drugs and proteins to the solid tumors and metastatic sites. However, there still must be sufficient loading of the bioactive, which is difficult particularly for essentially insoluble drugs. The difficulty is exponentially increased when attempting to co-load bioactive compounds into a single polymer nanoparticle. In embodiments where the surface of the nanoparticles is PEGylated, this can increase colloidal stability in circulation and reduce nonspecific uptake by the mononuclear phagocyte system (MPS). In some embodiments, these nanoparticles are also functionalized with anisamide (MBA), to target the sigma receptor over expressed on tumor cells to facilitate cellular uptake. The in vitro and in vivo performance of these nanoparticles can be characterized in terms of tumor-targeted delivery of the bioactive compounds. Additionally, systemic toxicity is examined to establish the safety of these nanoparticles.

As used herein, “high loading capacity” means an improved or better loading capacity of the active compound than any of the known nanoparticle formulations of that particular active compound.

As used herein, “high bioavailability” means a better or improved bioavailability of the bioactive compound in comparison to the bioavailability of the free bioactive compound. By “free bioactive compound” is meant a bioactive compound that is not a precipitate that is encapsulated with a lipid.

As used herein, “less toxicity” means less or not toxic in comparison to the free bioactive compound or any known formulation thereof.

As used herein, “higher rate of absorption” means a better or improved rate of absorption of the active compound in comparison to the free bioactive compound or any known formulation thereof.

As used herein, “improved efficacy” means efficacy of the bioactive compound that is better in kind or degree of both in comparison to any of the known nanoparticle formulations of that particular bioactive compound.

The above properties can be measured and quantified using any of the well-known methods in the art.

In an embodiment, the subject matter described herein is directed to a method of preparing a polymer nanoparticle comprising:
i. forming an organic phase by contacting a first lipid coated nano-precipitated core containing a first bioactive compound with, 1) a different hydrophobic bioactive compound in its free form and 2) a polymer; wherein said contacting is in an organic solvent that is miscible in water to form said organic phase; and
ii. contacting the organic phase with an aqueous solution to form said polymer nanoparticle comprising the first lipid coated core containing a first bioactive compound, and the hydrophobic bioactive compound.

In an embodiment, the subject matter described herein is directed to a method of preparing a polymer nanoparticle comprising:
i. forming an organic phase by contacting a first lipid coated nano-precipitated core containing a first bioactive compound with, 1) a second lipid coated nano-precipitated core containing a second bioactive compound that is different from the first bioactive compound and 2) a polymer; wherein said contacting is in an organic solvent that is miscible in water to form said organic phase; and
ii. contacting the organic phase with aqueous solution to form said polymer nanoparticle comprising the lipid coated core containing a first bioactive compound, and the lipid coated core containing a second bioactive compound that is different from the first bioactive compound.

The methods can further comprise rinsing and purifying the nanoparticles once formed. In embodiments, the aqueous solution is water, which can be deionized. In the above methods, the polymer may be contacted with the organic solvent first or the cores or the hydrophobic bioactive compound may be contacted first followed by the polymer. The methods can be conducted with heating to above room temperature or cooling to below room temperature or can be conducted at room temperature. The only limitation is that the components must be able to withstand the temperature.

In embodiments, the subject matter described herein is directed to polymer nanoparticles comprising ratiometric amounts of bioactive compounds that provide synergistic effects, wherein there is at least one lipid-coated nano-precipitated core containing a bioactive compound and at least one different bioactive compound in a free form or in a lipid-coated nano-precipitated core.

In embodiments, the subject matter described herein is directed to a nanoparticle whereby the presence of a first bioactive compound increases the loading efficiency of a second, third or more bioactive compound.

The nanoparticles described herein can be self-assembling, substantially spherical vesicles. The exterior of the nanoparticle comprises a polymer. Useful polymers include known polymers that are biocompatible. The term “biocompatible” is used herein as it is used in the art to describe polymers that are appropriate for pharmaceutical use. Biocapmatible polymers may be bioresorptive polymers that degrade and are absorbed by the body over time.

Polymer refers to a chemical compound or mixture of compounds formed by polymerization and consisting essentially of repeating structural units. Useful polymers can be synthetic materials used in vivo or in vitro that are capable of forming the nanoparticles and are intended to interact with a biological system. These include, but are not limited to those taught in U.S. Pat. No. 5,514,378 (incorporated herein by reference). Biodegradable copolymers have also been described, including aliphatic polyester, polyorthoester, polyanhydride, poly alpha-amino acid, polyphosphagen, and polyalkylcyanoacrylate. Among aliphatic polyesters, polylactide (PLA), polyglycolide (PGA) and polylactideglycolide (PLGA). Biodegradable polymers include lactic acid polymers such as poly(L-lactic acid) (PLLA), poly(DL-lactic acid) (PLA), and poly(DL-lactic-co-glycolic acid) (PLGA). The co-monomer (lactide:glycolide) ratios of the poly(DL-lactic-co-glycolic acid) are preferably between 100:0 and 50:50. Most preferably, the co-monomer ratios are between 85:15 (PLGA 85:15) and 50:50 (PLGA 50:50). Blends of PLLA with PLGA, preferably PLGA 85:15 and PLGA 50:50, can be used. A particularly useful polymer is poly(lactic-co-glycolic acid) (PLGA). The interior of the nanoparticle is encapsulated by the polymer(s) and comprises the bioactive compounds.

The nano-precipitated core comprising a bioactive compound can be lipid coated. As used herein, the term “lipid” refers to a member of a group of organic compounds that has lipophilic or amphipathic properties, including, but not limited to, fats, fatty oils, essential oils, waxes, steroids, sterols, phospholipids, glycolipids, sulpholipids, aminolipids, chromolipids (lipochromes), and fatty acids. The term “lipid” encompasses both naturally occurring and synthetically produced lipids. “Lipophilic” refers to those organic compounds that dissolve in fats, oils, lipids, and non-polar solvents, such as organic solvents. Lipophilic compounds are sparingly soluble or insoluble in water. Thus, lipophilic compounds are hydrophobic. Amphipathic lipids, also referred to herein as “amphiphilic lipids” refer to a lipid molecule having both hydrophilic and hydrophobic characteristics. The hydrophobic group of an amphipathic lipid, as described in more detail immediately herein below, can be a long chain hydrocarbon group. The hydrophilic group of an amphipathic lipid can include a charged group, e.g., an anionic or a cationic group, or a polar, uncharged group Amphipathic lipids can have multiple hydrophobic groups, multiple hydrophilic groups, and combinations thereof. Because of the presence of both a hydrophobic group and a hydrophilic group, amphipathic lipids can be soluble in water, and to some extent, in organic solvents.

As used herein, “hydrophilic” is a physical property of a molecule that is capable of hydrogen bonding with a water (H₂O) molecule and is soluble in water and other polar solvents. The terms “hydrophilic” and “polar” can be used interchangeably. Hydrophilic characteristics derive from the presence of polar or charged groups, such as carbohydrates, phosphate, carboxylic, sulfato, amino, sulfhydryl, nitro, hydroxy and other like groups.

Conversely, the term “hydrophobic” is a physical property of a molecule that is repelled from a mass of water and can be referred to as “nonpolar,” or “apolar,” all of which are terms that can be used interchangeably with “hydrophobic.” Hydrophobicity can be conferred by the inclusion of apolar groups that include, but are not limited to, long chain saturated and unsaturated aliphatic hydrocarbon groups and such groups substituted by one or more aromatic, cycloaliphatic or heterocyclic group(s). Examples of amphipathic compounds include, but are not limited to, phospholipids, aminolipids and sphingolipids. Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, dioleoyl phosphatidic acid, and dilinoleoylphosphatidylcholine. Other compounds lacking in phosphorus, such as sphingolipid, glycosphingolipid families, diacylglycerols and β-acyloxyacids, also are within the group designated as amphipathic lipids.

Lipids can include cationic lipids. As used herein, the term “cationic lipid” encompasses any of a number of lipid species that carry a net positive charge at physiological pH, which can be determined using any method known to one of skill in the art. Such lipids include, but are not limited to, the cationic lipids of formula (I) disclosed in International Application No. PCT/US2009/042476, entitled “Methods and Compositions Comprising Novel Cationic Lipids,” which was filed on May 1, 2009, and is herein incorporated by reference in its entirety. These include, but are not limited to, N-methyl-N-(2-(arginoylamino) ethyl)-N, N-Di octadecyl aminium chloride or di stearoyl arginyl ammonium chloride] (DSAA), N,N-di-myristoyl-N-methyl-N-2[N′—(N⁶-guanidino-L-lysinyl)] aminoethyl ammonium chloride (DMGLA), N,N-dimyristoyl-N-methyl-N-2[N²-guanidino-L-lysinyl] aminoethyl ammonium chloride, N,N-dimyristoyl-N-methyl-N-2[N′—(N2, N6-di-guanidino-L-lysinyl)] aminoethyl ammonium chloride, and N,N-di-stearoyl-N-methyl-N-2[N′—(N6-guanidino-L-lysinyl)] aminoethyl ammonium chloride (DSGLA). Other non-limiting examples of cationic lipids that can be present include N,N-dioleyl-N,N-dimethylammonium chloride (“DODAC”); N-(2,3-dioleoyloxy) propyl)-N,N,N-trimethylammonium chloride (“DOTAP”); N-(2,3-dioleyloxy) propyl)-N,N,N-trimethylammonium chloride (“DOTMA”) or other N—(N,N-1-dialkoxy)-alkyl-N,N,N-trisubstituted ammonium surfactants; N,N-distearyl-N,N-dimethylammonium bromide (“DDAB”); 3-(N—(N′,N′-dimethylaminoethane)-carbamoyl) cholesterol (“DC-Chol”) and N-(1,2-dimyristyloxyprop-3-yl)-N,N-dimethyl-N-hydroxyethyl ammonium bromide (“DMRIE”); 1,3-dioleoyl-3-trimethylammonium-propane, N-(1-(2,3-dioleyloxy)propyl)-N-(2-(sperminecarboxamido)ethyl)-N,N-dimethy-1 ammonium trifluoro-acetate (DOSPA); GAP-DLRIE; DMDHP; 3-β[⁴N—(¹N,⁸N-diguanidino spermidine)-carbamoyl] cholesterol (BGSC); 3-β[N,N-diguanidinoethyl-aminoethane)-carbamoyl] cholesterol (BGTC); N,N¹,N²,N³ Tetra-methyltetrapalmitylspermine (cellfectin); N-t-butyl-N′-tetradecyl-3-tetradecyl-aminopropion-amidine (CLONfectin); dimethyldioctadecyl ammonium bromide (DDAB); 1,3-dioleoyloxy-2-(6-carboxyspermyl)-propyl amide (DOSPER); 4-(2,3-bis-palmitoyloxy-propyl)-1-methyl-1H-imidazole (DPIM) N,N,N′,N′-tetramethyl-N,N′-bis(2-hydroxyethyl)-2,3 dioleoyloxy-1,4-butanediammonium iodide) (Tfx-50); 1,2 dioleoyl-3-(4′-trimethylammonio) butanol-sn-glycerol (DOBT) or cholesteryl (4′trimethylammonia) butanoate (ChOTB) where the trimethylammonium group is connected via a butanol spacer arm to either the double chain (for DOTB) or cholesteryl group (for ChOTB); DL-1,2-dioleoyl-3-dimethylaminopropyl-β-hydroxyethylammonium (DORI) or DL-1,2-O-dioleoyl-3-dimethylaminopropyl-β-hydroxyethylammonium (DORIE) or analogs thereof as disclosed in International Application Publication No. WO 93/03709, which is herein incorporated by reference in its entirety; 1,2-dioleoyl-3-succinyl-sn-glycerol choline ester (DOSC); cholesteryl hemisuccinate ester (ChOSC); lipopolyamines such as dioctadecylamidoglycylspermine (DOGS) and dipalmitoyl phosphatidylethanolamylspermine (DPPES) or the cationic lipids disclosed in U.S. Pat. No. 5,283,185, which is herein incorporated by reference in its entirety; cholesteryl-3β-carboxyl-amido-ethylenetrimethylammonium iodide; 1-dimethylamino-3-trimethylammonio-DL-2-propyl-cholesteryl carboxylate iodide; cholesteryl-3-β-carboxyamidoethyleneamine; cholesteryl-3-β-oxysuccinamido-ethylenetrimethylammonium iodide; 1-dimethylamino-3-trimethylammonio-DL-2-propyl-cholesteryl-3-β-oxysuccinate iodide; 2-(2-trimethylammonio)-ethylmethylamino ethyl-cholesteryl-3-β-oxysuccinate iodide; and 3-β-N-(polyethyleneimine)-carbamoylcholesterol.

The lipids can contain co-lipids that are negatively charged or neutral. As used herein, a “co-lipid” refers to a non-cationic lipid, which includes neutral (uncharged) or anionic lipids. The term “neutral lipid” refers to any of a number of lipid species that exist either in an uncharged or neutral zwitterionic form at physiological pH. The term “anionic lipid” encompasses any of a number of lipid species that carry a net negative charge at physiological pH. Co-lipids can include, but are not limited to, diacylphosphatidylcholine, diacylphosphatidylethanolamine, ceramide, sphingomyelin, cephalin, cholesterol, cerebrosides and diacylglycerols, phospholipid-related materials, such as lecithin, phosphatidylethanolamine, lysolecithin, lysophosphatidylethanolamine, phosphatidylserine, phosphatidylinositol, cardiolipin, phosphatidic acid, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), palmitoyloleyolphosphatidylglycerol (POPG), dipalmitoylphosphatidylglycerol (DPPG), dioleoyl-phosphatidylethanolamine (DOPE), palmitoyloleoylphosphatidylchol-ine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dioleoyl phosphatidic acid (DOPA), stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, lysophosphatidylcholine, and dioctadecyldimethyl ammonium bromide and the like. Co-lipids also include polyethylene glycol-based polymers such as PEG 2000, PEG 5000 and polyethylene glycol conjugated to phospholipids or to ceramides, as described in U.S. Pat. No. 5,820,873, herein incorporated by reference in its entirety.

Preferably, the amphiphilic lipid having a free phosphate group is dioleoyl phosphatidic acid (DOPA).

While not being bound by any particular theory or mechanism of action, it is believed that the nanoparticles can enter cells through endocytosis and are found in endosomes, which exhibit a relatively low pH (e.g., pH 5.0). Thus, in some embodiments, the bioactive compound is released at endosomal pH. In certain embodiments, the pH level is less than about 6.5, less than about 6.0, less than about 5.5, less than about 5.0, less than about 4.5, or less than about 4.0, including but not limited to, about 6.5, about 6.4, about 6.3, about 6.2, about 6.1, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, or less.

The nanoparticles can be of any size, so long as they are capable of delivering the incorporated bioactive compounds to a cell (e.g., in vitro, in vivo), physiological site, or tissue. As used herein, the term “nanoparticle” refers to particles of any shape having at least one dimension that is less than about 1000 nm. In some embodiments, nanoparticles have at least one dimension in the range of about 1 nm to about 1000 nm, including any integer value between 1 nm and 1000 nm (including about 1, 2, 5, 10, 20, 30, 40, 50, 60, 70, 80, 90, 100, 200, 500, and 1000). In certain embodiments, the nanoparticles have at least one dimension that is about 150 nm Spherical nanoparticles can have a diameter of less than about 100 nm, including but not limited to about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, and 100 nm. In particular embodiments, the nanoparticles have a diameter of less than about 50 nm. In particular embodiments, the nanoparticles have a diameter of between about 40 nm and about 50 nm. In particular embodiments, the nanoparticles have a zeta potential of about −17 mV. Particle size can be determined using any method known in the art, including, but not limited to, sedimentation field flow fractionation, photon correlation spectroscopy, disk centrifugation, and dynamic light scattering (using, for example, a submicron particle sizer such as the NICOMP particle sizing system from AutodilutePAT Model 370; Santa Barbara, Calif.).

The nano-precipitated cores are formed by an emulsion process. An emulsion is a dispersion of one liquid in a second immiscible liquid. The term “immiscible” when referring to two liquids refers to the inability of these liquids to be mixed or blended into a homogeneous solution. Two immiscible liquids when added together will always form two separate phases. The organic solvent used in the presently disclosed methods is essentially immiscible with water. Emulsions are essentially swollen micelles, although not all micellar solutions can be swollen to form an emulsion. Micelles are colloidal aggregates of amphipathic molecules that are formed at a well-defined concentration known as the critical micelle concentration. Micelles are oriented with the hydrophobic portions of the lipid molecules at the interior of the micelle and the hydrophilic portions at the exterior surface, exposed to water. The typical number of aggregated molecules in a micelle (aggregation number) has a range from about 50 to about 100. The term “micelles” also refers to inverse or reverse micelles, which are formed in an organic solvent, wherein the hydrophobic portions are at the exterior surface, exposed to the organic solvent and the hydrophilic portion is oriented towards the interior of the micelle.

An oil-in-water (O/W) emulsion consists of droplets of an organic compound (e.g., oil) dispersed in water and a water-in-oil (W/O) emulsion is one in which the phases are reversed and is comprised of droplets of water dispersed in an organic compound (e.g., oil). A water-in-oil emulsion is also referred to herein as a reverse emulsion. Thermodynamically stable emulsions are those that comprise a surfactant (e.g, an amphipathic molecule) and are formed spontaneously. The term “emulsion” can refer to microemulsions or macroemulsions, depending on the size of the particles. Droplet diameters in microemulsions typically range from about 10 to about 100 nm. In contrast, the term macroemulsions refers to droplets having diameters greater than about 100 nm.

It will be evident to one of skill in the art that sufficient amounts of the aqueous solutions, organic solvent, and surfactants can be added to the reaction solution to form the desired emulsion.

Surfactants are added to the reaction solution in order to facilitate the development of and stabilize the water-in-oil microemulsion. Surfactants are molecules that can reduce the surface tension of a liquid. Surfactants have both hydrophilic and hydrophobic properties, and thus, can be solubilized to some extent in either water or organic solvents. Surfactants are classified into four primary groups: cationic, anionic, non-ionic, and zwitterionic. Preferably, the surfactants are non-ionic surfactants. Non-ionic surfactants are those surfactants that have no charge when dissolved or dispersed in aqueous solutions. Thus, the hydrophilic moieties of non-ionic surfactants are uncharged, polar groups. Representative non-limiting examples of non-ionic surfactants suitable for use for the presently disclosed methods and compositions include polyethylene glycol, polysorbates, including but not limited to, polyethoxylated sorbitan fatty acid esters (e.g., Tween® compounds) and sorbitan derivatives (e,g., Span® compounds); ethylene oxide/propylene oxide copolymers (e.g., Pluronic® compounds, which are also known as poloxamers); polyoxyethylene ether compounds, such as those of the Brij® family, including but not limited to polyoxyethylene stearyl ether (also known as polyoxyethylene (100) stearyl ether and by the trade name Brij® 700); ethers of fatty alcohols. In particular embodiments, the non-ionic surfactant comprises octyl phenol ethoxylate (i.e., Triton X-100), which is commercially available from multiple suppliers (e.g., Sigma-Aldrich, St. Louis, Mo.).

Polyethoxylated sorbitan fatty acid esters (polysorbates) are commercially available from multiple suppliers (e.g., Sigma-Aldrich, St Louis, Mo.) under the trade name Tween®, and include, but are not limited to, polyoxyethylene (POE) sorbitan monooleate (Tween® 80), POE sorbitan monostearate (Tween® 60), POE sorbitan monolaurate (Tween® 20), and POE sorbitan monopalmitate (Tween® 40).

Ethylene oxide/propylene oxide copolymers include the block copolymers known as poloxamers, which are also known by the trade name Pluronic® and can be purchased from BASF Corporation (Florham Park, N.J.). Poloxamers are composed of a central hydrophobic chain of polyoxypropylene (poly(propylene oxide)) flanked by two hydrophilic chains of polyoxyethylene (poly(ethylene oxide)) and are represented by the following chemical structure: HO(C₂H₄O)ₐ(C₃H₆O)_{b}(C₂H₄O)ₐH; wherein the C₂H₄O subunits are ethylene oxide monomers and the C3H6O subunits are propylene oxide monomers, and wherein a and b can be any integer ranging from 20 to 150.

Organic solvents that can be used in the presently disclosed methods include those that are immiscible or essentially immiscible with water. A non-limiting example of an organic solvent that can be used in the presently disclosed methods is tetrahydrofuran (THF). In particular embodiments, the organic solvent is nonpolar or essentially nonpolar. In some embodiments, mixtures of more than one organic solvent can be used in the presently disclosed methods. Surfactants can be added to the solution.

The reaction solution may be mixed to form the microemulsion and the solution may also be incubated for a period of time. This incubation step can be performed at room temperature. In some embodiments, the reaction solution is mixed at room temperature for a period of time of between about 5 minutes and about 60 minutes, including but not limited to about 5 minutes, about 10 minutes, about 15 minutes, about 20 minutes, about 25 minutes, about 30 minutes, about 35 minutes, about 40 minutes, about 45 minutes, about 50 minutes, about 55 minutes, and about 60 minutes. In particular embodiments, the reaction solution is mixed at room temperature for about 15 minutes.

In order to complex the nano-precipitated bioactive compound with a lipid, the surface of the nano-precipitate can be charged, either positively or negatively. In some embodiments, the precipitate will have a charged surface following its formation. Those nano-precipitates with positively charged surfaces can be mixed with anionic lipids, whereas those nano-precipitates with negatively charged surfaces can be mixed with cationic lipids.

In certain embodiments, the surface charge of the nano-precipitate can be enhanced or reversed using any method known in the art. For example, a nano-precipitate having a positively charged surface can be modified to create a negatively charged surface. Alternatively, a nano-precipitate having a negatively charged surface can be modified to create a positively charged surface.

In those embodiments wherein a nano-precipitate is created having a positive surface charge, the surface charge can be made negative through the addition of sodium citrate to the water-in-oil microemulsion. In some embodiments, sodium citrate is added at a concentration of about 15 mM to the microemulsion. In some of these embodiments, the total volume of the 15 mM sodium citrate added to the microemulsion is about 125 μl. Sodium citrate is especially useful for imparting a negative surface charge to the nano-precipitates because it is non-toxic.

In some embodiments, the precipitate has or is modified to have a zeta potential of less than −10 mV and in certain embodiments, the zeta potential is between about −14 mV and about −20 mV, including but not limited to about −14 mV, about −15 mV, about −16 mV, about −17 mV, about −18 mV, about −19 mV, and about −20 mV. In particular embodiments, the zeta potential of the nano-precipitate is about −16 mV.

Following the production of the emulsion, nano-precipitated bioactive having a lipid coating can be purified from the non-ionic surfactant and organic solvent. The nano-precipitate can be purified using any method known in the art, including but not limited to gel filtration chromatography. A nano-precipitate that has been purified from the non-ionic surfactants and organic solvent is a nano-precipitate that is essentially free of non-ionic surfactants or organic solvents (e.g, the nano-precipitate comprises less than 10%, less than 1%, less than 0.1% by weight of the non-ionic surfactant or organic solvent). In some of those embodiments wherein gel filtration is used to purify the nano-precipitate, the precipitate is adsorbed to a silica gel or to a similar type of a stationary phase, the silica gel or similar stationary phase is washed with a polar organic solvent (e.g., ethanol, methanol, acetone, DMSO, DMF) to remove the non-ionic surfactant and organic solvent, and the nano-precipitate is eluted from the silica gel or other solid surface with an aqueous solution comprising a polar organic solvent.

In some of these embodiments, the silica gel is washed with ethanol and the nano-precipitate is eluted with a mixture of water and ethanol. In particular embodiments, the nano-precipitate is eluted with a mixture of water and ethanol, wherein the mixture comprises a volume/volume ratio of between about 1:9 and about 1:1, including but not limited to, about 1:9, about 1:8, about 1:7, about 1:6, about 1:5, about 1:4, about 1:3, about 1:2, and about 1:1. In particular embodiments, the volume/volume ratio of water to ethanol is about 1:3. In some of these embodiments, a mixture comprising 25 ml water and 75 ml ethanol is used for the elution step. Following removal of the ethanol using, for example, rotary evaporation, the nano-precipitate can be dispersed in an aqueous solution (e.g., water) prior to mixing with the organic solvent and the polymer to form the nanoparticles. In certain embodiments, the methods of making the nanoparticles can further comprise toher or an additional purification steps. Purification can be accomplished through any method known in the art, including, but not limited to, centrifugation through a sucrose density gradient or other media which is suitable to form a density gradient. It is understood, however, that other methods of purification such as chromatography, filtration, phase partition, precipitation or absorption can also be utilized.

A DOPA-coated calcium phosphate (CaP) platform can encapsulate phosphate-containing drugs including Gemcitabine Monophosphate, siRNA, and others (US Appl. Pub. No. 2012/0201872). As disclosed herein, multiple drugs can be encapsulated into PLGA NPs efficiency, allowing fine-tuning of the ratio of drug loading.

In an embodiment, encapsulated gemcitabine monophosphate CaP cores and CDDP cores in PLGA NPs can be used for the treatment of cancer. In particular, bladder cancer. Non-overlapping mechanisms of action increase the possibility of synergistic anti-cancer effects between these two drugs in the treatment of bladder cancer. A study of CDDP+GC combination therapy with cultured UMUC-3 (Human Bladder Transitional Cell Carcinoma) cells showed ratio-dependent synergistic effects on cell proliferation. With conventional administration, the ratio between two drugs accumulated within tumor tissues may differ from the initial administered ratio. Use of a single vehicle (liposomes or polymeric nanoparticles) for combination therapy provides an opportunity to address this problem of controlling drug ratios in vivo.

Double emulsion has been employed to encapsulate hydrophilic drugs into PLGA NPs. This method has been limited by low loading efficiency (<1.0%) and limited encapsulation efficiency. Incorporation of both poorly water-soluble and poorly oil soluble CDDP has been even more problematic. Drugs in either DOPA-CDDP cores or CaP cores (eg. DOPA-GMP cores) can be encapsulated into PLGA15k-PEG 3500 nanoparticles (PEG-PLGA NPs) through nanoprecipitation with ratiometric control (Scheme 3). A tethered targeting ligand anisamide (MBA) was further anchored onto PLGA15k-PEG 3500 NPs (MBA-PEG-PLGA NPs), which increased the tumor accumulation of the drug through the enhanced permeability and retention (EPR) effect and specific sigma receptor targeting mechanism.

Scheme 1 depicts a synthetic route for preparing exemplary nanoparticles.

Scheme 2 depicts a synthetic route for preparing MBA-PEG-PLGA.

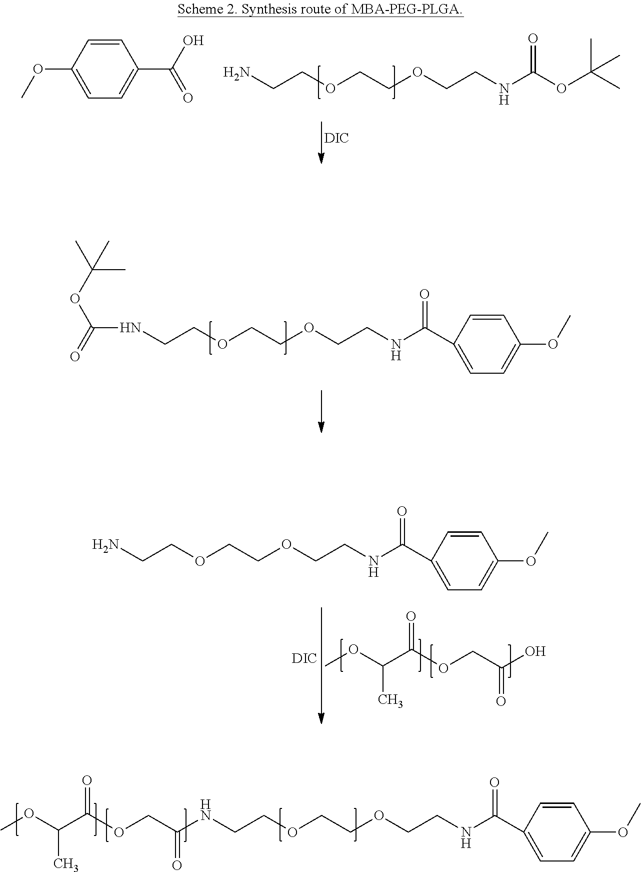

Scheme 3 depicts a synthetic route for preparing MBA-PEG-PLGA NPs containing DOPA-CDDP cores and DOPA-GMP cores.

Scheme 4 depicts the ratiometric loading and delivery of bioactive compounds, GMP and CDDP.

In some embodiments, the first reverse microemulsion has the same or different pH as the second reverse microemulsion.

In some embodiments, the nano-precipitate is washed with ethanol, and the washing step can be performed about 1-5 times, including 1, 2, 3, 4, and 5.

Bioactive compounds include those that can be combined with an ion species to form a nano-precipitate in salt form. Such useful bioactive compounds are disclosed in US Appl. Pub. No. 2012/0201872 and PCT/US2013/061985, each of which is herein incorporated in its entirety. Precursors can combine with a cation, such as In⁺³, Gd₊₃, Mg₊₂, Zn⁺² and Ba⁺² or an anion, such as a halide, to form a nano-precipitate in situ, i.e., during mixing of the reverse micro-emulsions. In the latter instance, preferably, the precursor is cis-diaminedihydroplatinum(II). Preferably, the bioactive compound that is in the form of a nano-precipitate is cisplatin or gemcitabine monophosphate. The latter can be a precipitated core containg calcium phosphate. Bioactive compounds that can be co-loaded include compounds in their free form that are hydrophobic. A preferred hydrophobic drug is rapamycin.

Other compounds will now be described. A “low solubility bioactive compound” is intended any agent that has a desired effect (e.g., therapeutic effect) on a living cell, tissue, or organism, or an agent that can desirably interact with a component (e.g., enzyme) of a living cell, tissue, or organism and that is not appreciably soluble in water and oil or a bioactive compound that can be soluble in water and/or oil, such as a precursor, that is capable of combining with an ion to form a nano-precipitate that is not appreciably solubilized in water and oil. The low solubility bioactive agents are also not appreciably solubilized under physiological conditions. Preferred bioactive agents can be formed into nano-precipitates and have a solubility of less than 10 mg/ml in water at 25° C. Unlike existing technologies, the subject matter described herein advantageously utilizes low-soluble or insoluble active agents and nano-precipitates thereof. Accordingly, it is preferred that the bioactive compound or its nano-precipitate has a solubility of less than 8 mg/ml in water at 25° C. More preferably, the bioactive compound or its nano-precipitate has a solubility of less than 5 mg/ml in water at 25° C. Most preferably, the bioactive compound or its nano-precipitate has a solubility of less than 3 mg/ml in water at 25° C.

In embodiments, low solubility bioactive compounds include compounds that are essentially insoluble in water and oil. The bioactive compounds useful in the polymer nanoparticles described herein combine with an ion (ionic species), e.g. an anion, such as a halide, or a cation, to form a nano-precipitate. In embodiments, the nano-precipitate consists essentially of the bioactive compound and the lipid. In other words, there is no other ionic core material present that is a seeding material.

It is noted that soluble bioactive compounds and, in particular, soluble precursor compounds can be utilized when they are prepared according to the methods described herein to form nano-precipitates as described herein. An example is the precursor of cisplatin that is combined with a halide salt to from a nano-precipitate. Another example is etoposide phosphate (Etopophos®), which is water soluble. However, using the methods described herein, etoposide phosphate contained in a first reverse emulsion can be contacted with InC13 contained in a second reverse emulsions. The In salt of etoposide phosphate formed therein is insoluble and formed a nano-precipitate.

Bioactive compounds can include, but are not limited to, polynucleotides, polypeptides, polysaccharides, organic and inorganic small molecules. The term “bioactive compound” encompasses both naturally occurring and synthetic bioactive compounds. The term “bioactive compound” can refer to a detection or diagnostic agent that interacts with a biological molecule to provide a detectable readout that reflects a particular physiological or pathological event.

Exemplary compounds include inorganic complexes such as platinum coordination complexes that include cisplatin, carboplatin, hydroxyurea, amsacrine, procarbazine, mitotane, mitoxantrone, levamisole, and hexamethylmelamine, paclitaxel.

Other specific bioactive compounds and their ion pairs that can form nano-precipitates are disclosed in US Appl. Pub. No. 2012/0201872 and PCT/US2013/061985, each of which is herein incorporated in its entirety. The essentially insoluble bioactive compound can be a chemotherapeutic drug. In other embodiments, the bioactive compound comprises a polynucleotide of interest or a polypeptide of interest, such as a silencing element (e.g., siRNA) as described elsewhere herein.

The bioactive compound can be a drug, including, but not limited to, antimicrobials, antibiotics, antimycobacterials, antifungals, antivirals, neoplastic agents, agents affecting the immune response, blood calcium regulators, agents useful in glucose regulation, anticoagulants, antithrombotics, antihyperlipidemic agents, cardiac drugs, thyromimetic and antithyroid drugs, adrenergics, antihypertensive agents, cholinergics, anticholinergics, antispasmodics, antiulcer agents, skeletal and smooth muscle relaxants, prostaglandins, general inhibitors of the allergic response, antihistamines, local anesthetics, analgesics, narcotic antagonists, antitussives, sedative-hypnotic agents, anticonvulsants, antipsychotics, anti-anxiety agents, antidepressant agents, anorexigenics, non-steroidal anti-inflammatory agents, steroidal anti-inflammatory agents, antioxidants, vaso-active agents, bone-active agents, antiarthritics, and diagnostic agents. Preferred antiviral drugs include tenofovir, adefovir, acyclovir monophosphate and L-thymidine monophosphate. In a preferred embodiment, the bioactive compound is an anticancer drug. In this embodiment, it is preferred that the bioactive compound is cisplatin and its analogues, etoposide monophosphate, alendronate, pamidronate, and gemcitabine monophosphate and salts, esters, conformers and produgs thereof.

As used herein, the term “deliver” refers to the transfer of a substance or molecule (e.g., a polynucleotide, bioactive compound, drug) to a physiological site, tissue, or cell. This encompasses delivery to the intracellular portion of a cell or to the extracellular space. As used herein, the term “intracellular” or “intracellularly” has its ordinary meaning as understood in the art. In general, the space inside of a cell, which is encircled by a membrane, is defined as “intracellular” space. Similarly, as used herein, the term “extracellular” or “extracellularly” has its ordinary meaning as understood in the art. In general, the space outside of the cell membrane is defined as “extracellular” space.

The methods disclosed herein provide for co-loading a nano-precipitate core and a hydrophobic bioactive compound or a hydrophobic bioactive compound derivative or analog. By the terms “derivative” or “analog,” is meant a chemically modified bioactive agent. For example, a bioactive compound can be made hydrophobic by methods known in the art. By making a hydrophobic analog or derivative of a bioactive compound, the bioactive compound can be employed in the present methods.

If the bioactive drug is hydrophobic, it can be co-loaded as a free drug. Alternatively, hydrophobic forms of a bioactive compound can be co-loaded.

Combination therapy is particularly effective in the treatment of HIV/AIDs and cancer. It provides a general means to enhance therapeutic efficacy, overcome treatment resistance, and diminish adverse effects. (F. Greco, M. J. Vicent, Adv. Drug Deliv. Rev. 2009, 61, 1203; b) H. J. Broxterman, N. H. Georgopapadakou, Drug Resist. Updat. 2005, 8, 183.) Adjustment of doses and molar ratios of combined drugs are used to promote synergistic rather than antagonistic effects. (a) L. D. Mayer, A. S. Janoff, Mol. Interv. 2007, 7, 216; b) L. D. Mayer, T. O. Harasym, P. G. Tardi, N. L. Harasym, C. R. Shew, S. A. Johnstone, E C Ramsay, M. B. Bally, A. S. Janoff, Mol. Cancer Ther. 2006, 5, 1854; c) P. G. Tardi, N. Dos Santos, T. O. Harasym, S. A. Johnstone, N. Zisman, A. W. Tsang, D. G. Bermudes, L. D. Mayer, Mol. Cancer Ther. 2009, 8, 2266.) However, differential pharmacokinetics and distribution of individual drugs within the conventionally administered “cocktail” lead to deviation from the optimized ratio during systemic delivery. This fact makes predicting improved in vivo therapeutic outcomes from in vitro synergistic effects a clinical challenge. (N. Kolishetti, S. Dhar, P. M. Valencia, L. Q. Lin, R. Karnik, S. J. Lippard, R. Langer, O. C. Farokhzad, Proc. Natl. Acad. Sci. USA 2010, 107, 17939.)

Nanomaterial-based delivery is one approach to unifying dual-drug pharmacokinetics. (Tardi, et. al). However, it is historically been difficult to load drugs with substantially different physical chemistry into the designed nano-carriers, which explains why only a few nanoparticulate formulations (Tardi, et. al). have been reported. Although attempts have been made, precise loading and ratiometric delivery of drugs with diverse solubility, steric configuration and other physicochemical properties still remains a challenge. (a) S. Aryal, C. M. Hu, L. Zhang, Mol. Pharm. 2011, 8, 1401; b) C. M. Hu, L. Zhang, Biochem. Pharmacol. 2012, 83, 1104). Moreover, combining individual therapeutic blocks together without interference their own functionalities adds to the complexity of compact nanostructures for combination drug delivery. (a) S. H. Hu, X. Gao, J. Am. Chem. Soc. 2010, 132, 7234; b) X. W. Chen, K. B. Sneed, S. Y. Pan, C. Cao, J. R. Kanwar, H. Chew, S. F. Zhou, Current drug metabolism 2012, 13, 640.

Cisplatin is considered the gold standard in several first-line combination therapies. (J. Valle, H. Wasan, D. H. Palmer, D. Cunningham, A. Anthoney, A. Maraveyas, S. Madhusudan, T. Iveson, S. Hughes, S. P. Pereira, M. Roughton, J. Bridgewater, N. Engl. J. Med. 2010, 362, 1273). A nanoparticulate approach used to enhance the ratio-dependent synergistic cisplatin-related combination therapy must overcome the difficulties in loading cisplatin along with other types of drugs into a single NP and the possible chemical interference with other groups of drugs such as nucleic acids. (a) S. M. Lee, T. V. O'Halloran, S. T. Nguyen, Journal of the American Chemical Society 2010, 132, 17130; b) X. Xu, K. Xie, X. Q. Zhang, E. M. Pridgen, G. Y. Park, D. S. Cui, J. Shi, J. Wu, P. W. Kantoff, S. J. Lippard, R. Langer, G. C. Walker, O. C. Farokhzad, Proceedings of the National Academy of Sciences of the United States of America 2013, 110, 18638). Limited solubility of inorganic cisplatin in both water and oil significantly hinders the development of NP with high drug loading and encapsulation efficacy. (S. Guo, Y. Wang, L. Miao, Z. Xu, C. M. Lin, Y. Zhang, L. Huang, ACS Nano 2013, 7, 9896.)

Gemcitabine monophosphate (GMP), an organic hydrophilic drug, and as described herein has been used for combination therapy with cisplatin. Gemcitabine is used as a first line therapy in combination with cisplatin for the treatment of bladder cancer. However, Gemcitabine relies on nucleoside transporters (J. R. Mackey, R. S. Mani, M. Selner, D. Mowles, J. D. Young, J. A. Belt, C. R. Crawford, C. E. Cass, Cancer research 1998, 58, 4349) to enter into cells where it is subsequently phosphorylated by deoxycytidine kinase to form active intermediates for DNA synthesis interference. GMP is one of the active intermediates of Gemcitabine. (W. Plunkett, P. Huang, V. Gandhi, Anti-cancer drugs 1995, 6 Suppl 6, 7). Since the addition of the first phosphate group in GMP formation is the rate-limiting step, GMP can be an efficient therapeutic drug candidate to demonstrate a synergistic effect in combination with cisplatin. (a) M. A. Moufarij, D. R. Phillips, C. Cullinane, Mol. Pharmacol. 2003, 63, 862; b) O. G. Besancon, G. A. Tytgat, R. Meinsma, R. Leen, J. Hoebink, G. V. Kalayda, U. Jaehde, H. N. Caron, A. B. van Kuilenburg, Cancer Lett. 2012, 319, 23). However, due to the significant difference in physicochemical properties, co-encapsulation of cisplatin and GMP would be expected to be problematic. As set forth herein, the NPs described herein can ratiometrically co-encapsulate and co-deliver native cisplatin and GMP while not compromising the drugs activities.

Dioleoyl phosphatidic acid (DOPA) coated calcium phosphate cores with the capability of loading hydrophilic phosphorylated drugs (such as GMP core) (Y. Zhang, W. Y. Kim, L. Huang, Biomaterials 2013, 34, 3447), small interfering RNA (siRNA) (J. Li, Y. Yang, L. Huang, J. Control. Release 2012, 158, 108), DNA (Y. Hu, M. T. Haynes, Y. Wang, F. Liu, L. Huang, ACS Nano 2013, 7, 5376) and peptides (Z. Xu, S Ramishetti, Y. C. Tseng, S. Guo, Y. Wang, L. Huang, J. Control. Release 2013, 172, 259); as well as DOPA coated cisplatin cores (CP core), where cisplatin serves as both nanocarrier and anti-cancer drug. (Guo et al., ACS Nano 2013; S. Guo, L. Miao, Y. Wang, L. Huang, J. Control. Release 2014, 174, 137). The surface and size similarities between these two categories of cores provide a methodology to unify a wide range of drugs or biomolecules with drastically disparate solubility and polarity into a standardized hydrophobic physicochemical property. As described herein, unifying physicochemical characteristics of dual drugs is a proposed prerequisite for ratio-controlled loading and delivery.

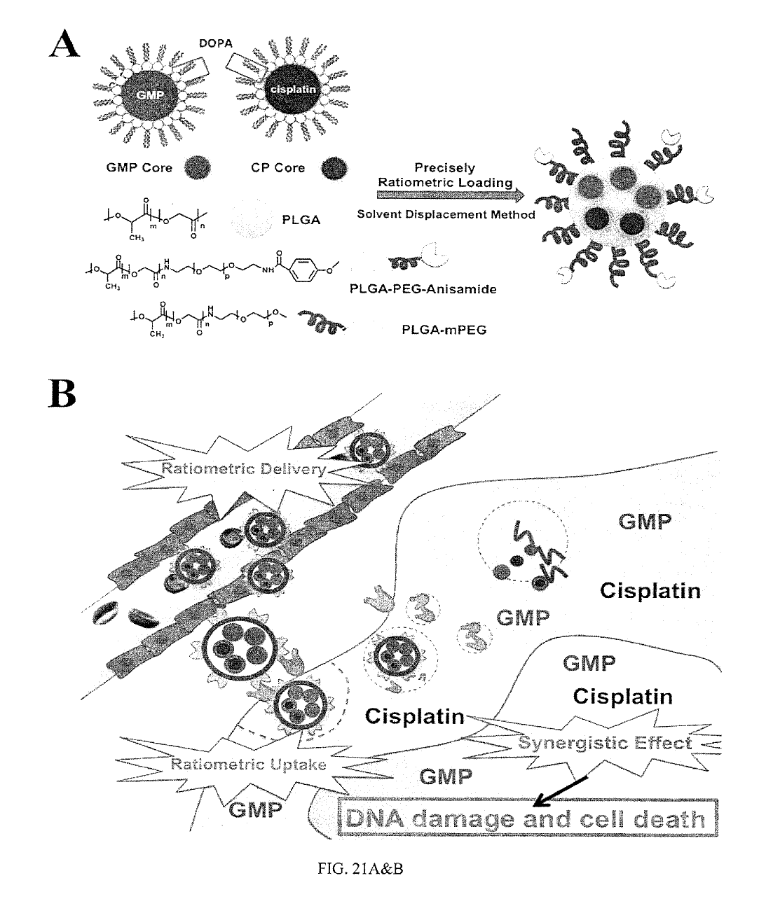

As disclosed herein, NPs have been prepared that provide both ratiometric loading and delivery of cisplatin with GMP. Cisplatin and GMP were formulated into DOPA coated CP cores and DOPA coated GMP cores. As shown in FIG. 21A, PLGA NP are used to incorporate these two separate hydrophobic cores. In this embodiment, CP cores and GMP cores have similar surface properties and these two drugs can be ratiometrically encapsulated into the same PLGA NP.

As described herein, CP cores and GMP cores were co-loaded into single PLGA NP using the solvent displacement method (FIG. 21A) and scheme 3. Ratiometric loading of GMP and cisplatin were determined and ratiometric loading property of PLGA NP was confirmed.

As described herein, this dual-drug containing NP can be ratiometrically delivered to the site of malignancy at the desired ratio (FIG. 21B). As described herein, the NPs were tested in vitro via release kinetics study and cellular uptake study, and in vivo via tumor accumulation analysis.

As described herein, co-delivery of both drugs at the desired ratio can result in synergistic anticancer efficacy. A stroma-rich human bladder cancer xenograft model was used to evaluate the anti-tumor efficacy of dual-drug containing NP at optimized ratio. (J. Zhang, L. Miao, S. Guo, Y. Zhang, L. Zhang, A. Satterlee, W. Y. Kim, L. Huang, J. Control. Release, DOI 10.1016/j.jconre1.2014.03.016). Synergistic anti-cancer effect was further determined via protein based mechanistic analysis. As described herein, cisplatin has been co-encapsulated with another hydrophilic drug in the same NP with precise ratiometric control.

The polymer nanoparticles described herein are useful in mammalian tissue culture systems, in animal studies, and for therapeutic purposes. The polymer nanoparticles comprising a bioactive compound having therapeutic activity when expressed or introduced into a cell can be used in therapeutic applications. The polymer nanoparticles can be administered for therapeutic purposes or pharmaceutical compositions comprising the polymer nanoparticles along with additional pharmaceutical carriers can be formulated for delivery, i.e., administering to the subject, by any available route including, but not limited, to parenteral (e.g., intravenous), intradermal, subcutaneous, oral, nasal, bronchial, opthalmic, transdermal (topical), transmucosal, rectal, and vaginal routes. In some embodiments, the route of delivery is intravenous, parenteral, transmucosal, nasal, bronchial, vaginal, and oral.

As used herein the term “pharmaceutically acceptable carrier” includes solvents, dispersion media, coatings, antibacterial and antifungal agents, isotonic and absorption delaying agents, and the like, compatible with pharmaceutical administration. Supplementary active compounds also can be incorporated into the compositions.

As one of ordinary skill in the art would appreciate, a presently disclosed pharmaceutical composition is formulated to be compatible with its intended route of administration. Solutions or suspensions used for parenteral (e.g., intravenous), intramuscular, intradermal, or subcutaneous application can include the following components: a sterile diluent such as water for injection, saline solution, fixed oils, polyethylene glycols, glycerine, propylene glycol or other synthetic solvents; antibacterial agents, such as benzyl alcohol or methyl parabens; antioxidants, such as ascorbic acid or sodium bisulfite; chelating agents, such as ethylenediaminetetraacetic acid; buffers, such as acetates, citrates or phosphates; and agents for the adjustment of tonicity, such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions or dispersions such as those described elsewhere herein and sterile powders for the extemporaneous preparation of sterile injectable solutions or dispersions. For intravenous administration, suitable carriers include physiological saline, bacteriostatic water, or phosphate buffered saline (PBS). The composition should be sterile and should be fluid to the extent that easy syringability exists. In some embodiments, the pharmaceutical compositions are stable under the conditions of manufacture and storage and should be preserved against the contaminating action of microorganisms, such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In some embodiments, isotonic agents, for example, sugars, polyalcohols, such as manitol or sorbitol, or sodium chloride are included in the formulation. Prolonged absorption of the injectable formulation can be brought about by including in the formulation an agent that delays absorption, for example, aluminum monostearate and gelatin.

Sterile injectable solutions can be prepared by filter sterilization as described elsewhere herein. In certain embodiments, solutions for injection are free of endotoxin. Generally, dispersions are prepared by incorporating the polymer nanoparticles into a sterile vehicle which contains a basic dispersion medium and the required other ingredients from those enumerated above. In those embodiments in which sterile powders are used for the preparation of sterile injectable solutions, the solutions can be prepared by vacuum drying and freeze-drying which yields a powder of the active ingredient plus any additional desired ingredient from a previously sterile-filtered solution thereof.

Oral compositions generally include an inert diluent or an edible carrier. Oral compositions can be prepared using a fluid carrier for use as a mouthwash. Pharmaceutically compatible binding agents, and/or adjuvant materials can be included as part of the composition. The oral compositions can include a sweetening agent, such as sucrose or saccharin; or a flavoring agent, such as peppermint, methyl salicylate, or orange flavoring.

For administration by inhalation, the presently disclosed compositions can be delivered in the form of an aerosol spray from a pressured container or dispenser which contains a suitable propellant, e.g., a gas such as carbon dioxide, or a nebulizer. Liquid aerosols, dry powders, and the like, also can be used.

Systemic administration of the presently disclosed compositions also can be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical or cosmetic carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of individuals. Guidance regarding dosing is provided elsewhere herein.

The present invention also includes an article of manufacture providing a polymer nanoparticle described herein. The article of manufacture can include a vial or other container that contains a composition suitable for the present method together with any carrier, either dried or in liquid form. The article of manufacture further includes instructions in the form of a label on the container and/or in the form of an insert included in a box in which the container is packaged, for carrying out the method of the invention. The instructions can also be printed on the box in which the vial is packaged. The instructions contain information such as sufficient dosage and administration information so as to allow the subject or a worker in the field to administer the pharmaceutical composition. It is anticipated that a worker in the field encompasses any doctor, nurse, technician, spouse, or other caregiver that might administer the composition. The pharmaceutical composition can also be self-administered by the subject.

The delivery of a bioactive compound to a cell can comprise an in vitro approach, an ex vivo approach, in which the delivery of the bioactive compound into a cell occurs outside of a subject (the transfected cells can then be transplanted into the subject), and an in vivo approach, wherein the delivery occurs within the subject itself.

The polymer nanoparticles described herein comprising a bioactive compound can be used for the treatment of a disease or unwanted condition in a subject, wherein the bioactive compound has therapeutic activity against the disease or unwanted condition when expressed or introduced into a cell. The bioactive compound is administered to the subject in a therapeutically effective amount. In those embodiments wherein the bioactive compound comprises a polynucleotide, when the polynucleotide of interest is administered to a subject in therapeutically effective amounts, the polynucleotide of interest or the polypeptide encoded thereby is capable of treating the disease or unwanted condition.

By “therapeutic activity” when referring to a bioactive compound is intended that the molecule is able to elicit a desired pharmacological or physiological effect when administered to a subject in need thereof.

As used herein, the terms “treatment” or “prevention” refer to obtaining a desired pharmacologic and/or physiologic effect. The effect may be prophylactic in terms of completely or partially preventing a particular infection or disease or sign or symptom thereof and/or may be therapeutic in terms of a partial or complete cure of an infection or disease and/or adverse effect attributable to the infection or the disease. Accordingly, the method “prevents” (i.e., delays or inhibits) and/or “reduces” (i.e., decreases, slows, or ameliorates) the detrimental effects of a disease or disorder in the subject receiving the compositions of the invention. The subject may be any animal, including a mammal, such as a human, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

The disease or unwanted condition to be treated can encompass any type of condition or disease that can be treated therapeutically. In some embodiments, the disease or unwanted condition that is to be treated is a cancer. As described elsewhere herein, the term “cancer” encompasses any type of unregulated cellular growth and includes all forms of cancer. In some embodiments, the cancer to be treated is a metastatic cancer. In particular, the cancer may be resistant to known therapies. Methods to detect the inhibition of cancer growth or progression are known in the art and include, but are not limited to, measuring the size of the primary tumor to detect a reduction in its size, delayed appearance of secondary tumors, slowed development of secondary tumors, decreased occurrence of secondary tumors, and slowed or decreased severity of secondary effects of disease.

It will be understood by one of skill in the art that the polymer nanoparticles can be used alone or in conjunction with other therapeutic modalities, including, but not limited to, surgical therapy, radiotherapy, or treatment with any type of therapeutic agent, such as a drug. In those embodiments in which the subject is afflicted with cancer, the polymer nanoparticles can be delivered in combination with any chemotherapeutic agent well known in the art.

The polymer surface of the nanoparticles can be PEGylated. The term “polymer-PEG conjugate” also refers to these polymer-PEG-targeting ligand conjugates and nanoparticles comprising a polymer-PEG targeting ligand conjugate. PEGylation enhances the circulatory half-life by reducing clearance of the nanoparticles by the reticuloendothelial (RES) system.

In some of those embodiments, the surface comprises a polymer-PEG conjugate at a concentration of about 4 mol % to about 15 mol % of the surface, including, but not limited to, about 4 mol %, about 5 mol %, about 6 mol %, about 7 mol %, 8 mol %, about 9 mol %, about 10 mol %, about 11 mol %, about 12 mol %, about 13 mol %, about 14 mol %, and about 15 mol % PEG. Higher percentage values (expressed in mol %) of PEG have also been found to be useful. Useful mol % values include those from about 12 mol % to about 50 mol %. Preferably, the values are from about 15 mol % to about 40 mol %. Also preferred are values from about 15 mol % to about 35 mol %. Most preferred values are from about 20 mol % to about 25 mol %, for example 23 mol %.

The polyethylene glycol moiety of the lipid-PEG conjugate can have a molecular weight ranging from about 100 to about 20,000 g/mol, including but not limited to about 100 g/mol, about 200 g/mol, about 300 g/mol, about 400 g/mol, about 500 g/mol, about 600 g/mol, about 700 g/mol, about 800 g/mol, about 900 g/mol, about 1000 g/mol, about 5000 g/mol, about 10,000 g/mol, about 15,000 g/mol, and about 20,000 g/mol. In some embodiments, the lipid-PEG conjugate comprises a PEG molecule having a molecular weight of about 2000 g/mol. In certain embodiments, the lipid-PEG conjugate comprises DSPE-PEG₂₀₀₀.

In some embodiments, the surface comprises a targeting ligand, thereby forming a targeted nanoparticle. By “targeting ligand” is intended a molecule that targets a physically associated molecule or complex to a targeted cell or tissue. As used herein, the term “physically associated” refers to either a covalent or non-covalent interaction between two molecules.

Targeting ligands can include, but are not limited to, small molecules, peptides, lipids, sugars, oligonucleotides, hormones, vitamins, antigens, antibodies or fragments thereof, specific membrane-receptor ligands, ligands capable of reacting with an anti-ligand, fusogenic peptides, nuclear localization peptides, or a combination of such compounds. Non-limiting examples of targeting ligands include asialoglycoprotein, insulin, low density lipoprotein (LDL), folate, benzamide derivatives, peptides comprising the arginine-glycine-aspartate (RGD) sequence, and monoclonal and polyclonal antibodies directed against cell surface molecules. In some embodiments, the small molecule comprises a benzamide derivative. In some of these embodiments, the benzamide derivative comprises anisamide.

Some targeting ligands comprise an intervening molecule in between the surface and the targeting ligand, which is covalently bound to both the surface and the targeting ligand. In some of these embodiments, the intervening molecule is polyethylene glycol (PEG).

By “targeted cell” is intended the cell to which a targeting ligand recruits a physically associated molecule or complex. The targeting ligand can interact with one or more constituents of a target cell. The targeted cell can be any cell type or at any developmental stage, exhibiting various phenotypes, and can be in various pathological states (i.e., abnormal and normal states). For example, the targeting ligand can associate with normal, abnormal, and/or unique constituents on a microbe (i.e., a prokaryotic cell (bacteria), viruses, fungi, protozoa or parasites) or on a eukaryotic cell (e.g., epithelial cells, muscle cells, nerve cells, sensory cells, cancerous cells, secretory cells, malignant cells, erythroid and lymphoid cells, stem cells). Thus, the targeting ligand can associate with a constitutient on a target cell which is a disease-associated antigen including, for example, tumor-associated antigens and autoimmune disease-associated antigens. Such disease-associated antigens include, for example, growth factor receptors, cell cycle regulators, angiogenic factors, and signaling factors.

In some embodiments, the targeting ligand interacts with a cell surface protein on the targeted cell. In some of these embodiments, the expression level of the cell surface protein that is capable of binding to the targeting ligand is higher in the targeted cell relative to other cells. For example, cancer cells overexpress certain cell surface molecules, such as the HER2 receptor (breast cancer) or the sigma receptor. In certain embodiments wherein the targeting ligand comprises a benzamide derivative, such as anisamide, the targeting ligand targets the associated polymer nanoparticles to sigma-receptor overexpressing cells, which can include, but are not limited to, cancer cells such as small- and non-small-cell lung carcinoma, renal carcinoma, colon carcinoma, sarcoma, breast cancer, melanoma, glioblastoma, neuroblastoma, and prostate cancer (Aydar, Palmer, and Djamgoz (2004) Cancer Res. 64:5029-5035).

Thus, in some embodiments, the targeted cell comprises a cancer cell. The terms “cancer” or “cancerous” refer to or describe the physiological condition in mammals that is typically characterized by unregulated cell growth. As used herein, “cancer cells” or “tumor cells” refer to the cells that are characterized by this unregulated cell growth. The term “cancer” encompasses all types of cancers, including, but not limited to, all forms of carcinomas, melanomas, sarcomas, lymphomas and leukemias, including without limitation, bladder carcinoma, brain tumors, breast cancer, cervical cancer, colorectal cancer, esophageal cancer, endometrial cancer, hepatocellular carcinoma, laryngeal cancer, lung cancer, osteosarcoma, ovarian cancer, pancreatic cancer, prostate cancer, renal carcinoma and thyroid cancer. In some embodiments, the targeted cancer cell comprises a lung cancer cell. The term “lung cancer” refers to all types of lung cancers, including but not limited to, small cell lung cancer (SCLC), non-small-cell lung cancer (NSCLC, which includes large-cell lung cancer, squamous cell lung cancer, and adenocarcinoma of the lung), and mixed small-cell/large-cell lung cancer. In particular, the nanoparticles are for use against melanomas.

In some embodiments, particularly those in which the diameter of the polymer nanoparticles is less than 100 nm, the polymer nanoparticles can be used to deliver bioactive compounds across the blood-brain barrier (BBB) into the central nervous system or across the placental barrier. Non-limiting examples of targeting ligands that can be used to target the BBB include transferring and lactoferrin (Huang et al. (2008) Biomaterials 29(2):238-246, which is herein incorporated by reference in its entirety). Further, the polymer nanoparticles can be transcytosed across the endothelium into both skeletal and cardiac muscle cells. For example, exon-skipping oligonucleotides can be delivered to treat Duchene muscular dystrophy (Moulton et al. (2009) Ann N Y Acad Sci 1175:55-60, which is herein incorporated by reference in its entirety).

Delivery of a therapeutically effective amount of polymer nanoparticles can be obtained via administration of a pharmaceutical composition comprising a therapeutically effective dose of the bioactive compound or the nanoparticles. By “therapeutically effective amount” or “dose” is meant the concentration of a delivery system or a bioactive compound comprised therein that is sufficient to elicit the desired therapeutic effect.

As used herein, “effective amount” is an amount sufficient to effect beneficial or desired clinical or biochemical results. An effective amount can be administered one or more times.

The effective amount of the polymer nanoparticles or bioactive compound will vary according to the weight, sex, age, and medical history of the subject. Other factors which influence the effective amount can include, but are not limited to, the severity of the subject's condition, the disorder being treated, the stability of the compound or complex, and, if desired, the adjuvant therapeutic agent being administered along with the polynucleotide delivery system. Methods to determine efficacy and dosage are known to those skilled in the art. See, for example, Isselbacher et al. (1996) Harrison's Principles of Internal Medicine 13 ed., 1814-1882, herein incorporated by reference.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic (e.g., immunotoxic) and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the presently disclosed methods, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans Levels in plasma can be measured, for example, by high performance liquid chromatography.

The pharmaceutical formulation can be administered at various intervals and over different periods of time as required, e.g., multiple times per day, daily, every other day, once a week for between about 1 to 10 weeks, between 2 to 8 weeks, between about 3 to 7 weeks, about 4, 5, or 6 weeks, and the like. The skilled artisan will appreciate that certain factors can influence the dosage and timing required to effectively treat a subject, including but not limited to the severity of the disease, disorder, or unwanted condition, previous treatments, the general health and/or age of the subject, and other diseases or unwanted conditions present. Generally, treatment of a subject can include a single treatment or, in many cases, can include a series of treatments. Further, treatment of a subject can include a single cosmetic application or, in some embodiments, can include a series of cosmetic applications.

It is understood that appropriate doses of a compound depend upon its potency and can optionally be tailored to the particular recipient, for example, through administration of increasing doses until a preselected desired response is achieved. It is understood that the specific dose level for any particular animal subject can depend on a variety of factors including the activity of the specific compound employed, the age, body weight, general health, gender, and diet of the subject, the time of administration, the route of administration, the rate of excretion, any drug combination, and the degree of expression or activity to be modulated.

One of ordinary skill in the art upon review of the presently disclosed subject matter would appreciate that the presently disclosed compounds and pharmaceutical compositions thereof, can be administered directly to a cell, a cell culture, a cell culture medium, a tissue, a tissue culture, a tissue culture medium, and the like. When referring to the delivery systems of the invention, the term “administering,” and derivations thereof, comprises any method that allows for the compound to contact a cell. The presently disclosed compounds or pharmaceutical compositions thereof, can be administered to (or contacted with) a cell or a tissue in vitro or ex vivo. The presently disclosed compounds or pharmaceutical compositions thereof, also can be administered to (or contacted with) a cell or a tissue in vivo by administration to an individual subject, e.g., a patient, for example, by systemic administration (e.g., intravenous, intraperitoneal, intramuscular, subdermal, or intracranial administration) or topical application, as described elsewhere herein.

The following examples are offered by way of illustration and not by way of limitation.

### EXAMPLES

### Examples 1-4

### Materials and Methods

tBOC-PEG₃₅₀₀-NH₂. HCl and mPEG₃₀₀₀-NH₂. HCl were ordered from JenKem Technology USA Inc. Acid terminated PLGA was purchased from DURECT Corporation. Cisplatin (CDDP) was purchased from Acros Organics. p-Anisic acid, EDC, NHS DIPEA and dichloromethane were obtained from Sigma-Aldrich. Luc-siRNA was purchased from Sigma-Aldrich, and Rapamycin was purchased from ChemieTek.

### Cell Lines:

A375M cells were cultured with RPMI 1640 medium (Gibco).

### Synthesis of PLGA-PEG-MBA:

800 mg Boc-PEG-NH₂.TFA, 278 mg Anisic acid (8 euqi) and 160 ul DIPEA (4 euqi) were dissolved in 10 ml of DCM. 283 μl DIC (8 euqi) was added into the mixture. 26 h later, MBA-PEG-NH2-Boc was purified by precipitation into ether and washed by ether. Yield: 530 mg, 66 wt %. The purity of MBA-PEG-Boc was confirmed using ¹H NMR. The amine group was quantatively consumed.

520 mg Boc-PEG-MBA was dissolved in 4.5 ml of TFA/DCM (1:2, v/v) mixture at room temperature. Two hours later, the solvent is removed under vacuum. The precipitate is re-dissolved in DCM and precipitated into ether. The solid compound is then washed by ether and dried under vacuum. Yield: 470 mg, 90 wt %. NMR spectra show that Boc group was completely de-protected.

445 mg MBA-PEG-NH₂.TFA (3500, 0.131 mmol), 1500 mg PLGA (15 kDa, 0.1 mmol) and 132 μl DIPEA (0.504 mmol) were dissolved in 6 ml of DCM. 250 μl DIC (1.0 mmol) was added into the mixture. Twenty-six hours later, the polymer was then purified by precipitation in methanol and washed by methanol. Yield: 1200 mg, 62 wt %. The structure of MBA-PEG-PLGA was confirmed by ¹H NMR.

Synthesis of mPEG-PLGA:

378 mg mPEG-NH₂.TFA (3000, 0.126 mmol), 1500 mg PLGA (15 kDa, 0.1 mmol) and 88 μl DIPEA (0.5 mmol) were dissolved in 6 ml DCM. 156 μl DIC (1.0 mmol) was added into the mixture. 24 h later, it was purified by precipitation into methanol, washed by methanol. Yield: 1200 mg, 64 wt %.

### Preparation of DOPA-CDDP Cores:

First, 100 μL of 200 mM cis-[Pt(NH₃)₂(H₂O)₂](NO₃)₂ was dispersed in a solution composed of mixture of cyclohexane/Igepal CO-520 (71:29, V:V) and cyclohexane/triton-X100/hexanol (75:15:10, V:V:V) to form a well-dispersed, water-in-oil reverse micro-emulsion. Another emulsion containing KCl was prepared by adding 100 μL of 800 mM KCl in water into a separate 8.0 mL oil phase. One hundred μL of DOPA (20 mM) was added to the CDDP precursor phase and the mixture was stirred. Twenty minutes later, the two emulsions were mixed and the reaction proceeded for another 30 min After that, 16.0 mL of ethanol was added to the micro-emulsion and the mixture was centrifuged at 12,000 g for at least 15 min to remove the cyclohexane and surfactants. After being extensively washed with ethanol 2-3 times, the pellets were re-dispersed in 3.0 ml of chloroform and stored in a glass vial for further modification.

### Preparation of DOPA-GMP Cores:

To prepare the DOPA-GMP cores, 100 μL 60 mM GMP was mixed with 500 μL 25 mM Na₂HPO₄ and then dispersed in 20 mL oil phase containing cyclohexane/Igepal CO-520 (71:29, V:V), while the other emulsion contained 600 μL 2.5 M CaCl₂. Six-hundred mL of 20 mM DOPA in chloroform was added to the phosphate phase. The two separate micro-emulsions were then mixed and stirred for 5 min. Another 400 mL of 20 mM DOPA was added into the emulsion. The emulsion was continually stirred for another 20 min before 40 mL of absolute ethanol was added. After that, the mixture was centrifuged at 12,000 g for at least 15 min to remove the cyclohexane and surfactants. After being extensively washed with ethanol 2-3 times, the pellets were re-dispersed in 2.0 ml of chloroform and stored in a glass vial for further modification.

Preparation of PLGA/PLGA-PEG-MBA (1:1 wt/wt) NPs Loaded with Different Cargos:

The cores and/or drugs were loaded into the MBA-PEG-PLGA NPs using a nanoprecipitation method described herein. The drugs and 10 mg of polymers were dissolved in 200 μl of THF and added dropwise into 2 ml of water under stirring at room temperature. The resulting NP suspension was allowed to stir uncovered for 6 h at room temperature to remove THF. The NPs were further purified by ultrafiltration (15 min, 3000×g, Amicon Ultra, Ultracel membrane with 50,000 NMWL, Millipore, Billerica, Mass.). Then, the PLGA-PEG NPs were re-suspended, washed with water, and collected likewise. To confirm the targeting ability of anisamide, 0.1 wt % of DiI was incorporated into PEG-PLGA NPs as a fluorescent probe.

### Characterization of Nanoparticles (NPs):

The loading and encapsulation efficiency of Rapamycin was measured using High-performance Liquid Chromatography (HPLC, Waters, λ=277 nm); the loading and encapsulation efficiency of CDDP was measured using Inductively Coupled Plasma-Mass Spectroscopy (ICP-MS, NexION™ 300, Perkin Elmer Inc); the loading efficiency of GMP was measured both by Ultraviolet-visible spectrometer (UV, DU®800, Beckman Coulter) and ³H labeled CMP incorporation using a Liquid Scintillation Analyzer (TRI-CARB 2900 TR, Packard Bioscience Co). The size distribution of particle was determined using a Malvern ZetaSizer Nano series (Westborough, Mass.). TEM images of NPs were acquired using a JEOL 100CX II TEM (JEOL, Japan). The NPs were negatively stained with 2% uranyl acetate. Approximately 100 NPs were analyzed for calculating the average number of CDDP cores per PLGA NP.

### Cellular Uptake:

NP uptake in cells was also measured using ICP-MS. A375M-Luc cells were seeded into a 12-well plate (1.5×10⁵ cells/well) containing 1 ml of media. Twenty-four hours later, 1 ml of free drug, targeted PLGA NPs containing CDDP alone or combination and non-targeted PLGA NPs containing CDDP alone or combination at a concentration of 20 μM CDDP were incubated with cells. After four hours, the cells were treated with lysis buffer. The concentration of CDDP was measured using ICP-MS.

In Vitro Release of CDDP and Rapamycin from PLGA NPs:

Rapamycin concentration was determined by high-performance liquid chromatography (HPLC), using CLC-ODS-18 column (5 cm, 4.6×150 mm; Waters Corporation, Milford, Mass.) maintained at 25° C., with an ultraviolet detector at 277 nm. The mixture of 60% acetonitrile and 40% water (v/v) was used as a mobile phase, and delivered at a flow rate of 0.5 mL min⁻¹ The injection volume was 20 μL and the retention time was about 5 min. In addition, Pt concentrations were measured using ICP-MS.

The dialysis technique was employed to study the release of Rapamycin and CDDP from different PLGA NPs in phosphate buffered saline (PBS) (pH 7.4) with 0.25% Tween-80. Rapamycin-loaded micelles with a final Rapamycin concentration of 0.85 mg/mL were placed into a dialysis tube with a molecular weight cutoff of 3000 Da, and dialyzed against 15 mL PBS (pH 7.4) with 0.25% Tween-80 in a thermo-controlled shaker with a stirring speed of 200 rpm at 37° C. Samples of 200 μL were withdrawn at specified times. Rapamycin concentration was determined by RP-HPLC; Pt concentrations were measured using ICP-MS.

The samples taken for measurement were replaced with fresh media and the cumulative amount of drug released into the media at each time point was calculated as the percentage of total drug released to the initial amount of the drug. All experiments were performed in duplicate and the data reported as the mean of three individual experiments.

In Vitro Release of CDDP and GMP from PLGA NPs:

In vitro release of GMP and CDDP from PLGA NPs was carried out using the same dialysis technique under same pH and temperature condition as mentioned above except that no Tween-80 was added into the system. Five hundred μL PLGA NPs loaded with 100 μg/mL GMP and CDDP separately or co-loaded with 100 ug/mL GMP and CDDP at ratio 5:1 were added into the dialysis bag and dialyzed for 96 h. In the preparation of DOPA-GMP core, a trace amount of radioactive cytidine 5′ monophosphate (CMP) [5-3H] disodium salt (Moravek Bio Inc, 1 mCi/mL) was mixed with GMP and served as a marker for the entrapped GMP. At each predetermined time point (1 h, 2 h, 4 h, 8 h, 12 h, 24 h, 36 h, 48 h and 96 h), four hundred μL samples were withdrawn and replaced with fresh media. Pt concentration and GMP concentrations were then determined by ICP-MS and scintilation analyzer respectively at specified times. All experiments were performed in triplicate and the data reported as the mean±SD of three individual experiments

### Cell Viability:

Cells were seeded in 96-well plates for 24 h at a density of 2×10³ cells/well. Cell masses that were viable after 2 days of drug exposure were determined by MTS assay. A CellTiter 96 AQueous One Solution Cell Proliferation Assay (Promega, Madison, Wis.) kit containing the tetrazolium compound MTS was used to assay cell viability according to the manufacturer's protocols.

### Drug Combination Analysis:

Drug combination analysis was performed by using the method described by Chou and Talalay.

### Apoptosis Assays:

Quantification of apoptosis by Annexin V/propidium iodide (PI) staining was performed using a BD ApoAlert annexin V-FITC Apoptosis Kit (BD Biosciences, USA) according to the manufacturer's instructions. Both floating and attached cells were collected 24 h after drug treatment. The concentration of RAPA was 0.36 μM and the concentration of CDDP was 2.0 μM. The molar ratio of CDDP to RAPA was 5.5. Cells were analyzed on a FACS Calibur flow cytometer using CellQuest Pro software (version 5.1.1; BD Biosciences, USA).

### In Vivo Anticancer Efficacy:

Five million A375M cells were injected subcutaneously into female athymic nude mice, 5-6 weeks old and weighing 18-22 g. After 8 days, the mice were randomly divided into four groups (4 mice per group). The mice were treated with weekly IV injections of PLGA NPs and saline as a control. A dose of 0.30 mg/kg of Pt and 0.15 mg/kg of Rapamycin was administered. Thereafter, tumor growth and body weight were monitored. Tumor volume was calculated using the following formula: TV=(L×W²)/2, with W being smaller than L Finally, mice were sacrificed using a CO₂ inhalation method. After the therapeutic experiment was done, major organs were collected after treatment and were formalin fixed and processed for routine H&E staining using standard methods Images were collected using a Nikon light microscope (Nikon). For the preliminary in vivo anticancer study of PLGA NPs co-loaded with DOPA-GMP and DOPA-CDDP cores, experiments were performed on nude mice bearing a combination of UMUC-3 Human Bladder Transitional Cell

### Carcinoma and NIH/3T3 Mouse Embryo Fibroblast Xenograft:

After 8 days, the mice were randomly divided into four groups (2 mice per group). The mice were treated IV injections of PLGA NPs and saline as a control for three times in total. A dose of 1.6 mg/kg of Pt and 8.0 mg/kg of GMP was administered. Tumor growth and body weight were monitored similarly as mentioned above.

### Masson Trichrome Staining:

Paraffin-embedded tumor sections was deparaffinized and rehydrated. The slides were then stained using Masson Trichrome kit (Sigma-Aldrich) according to manufacture instructions.

### TUNEL Assay:

The tumors were fixed in 4.0% paraformaldehyde (PFA), paraffin-embedded, and sectioned at the UNC Lineberger Comprehensive Cancer Center Animal Histopathology Facility. To detect apoptotic cells in tumor tissues, a TUNEL assay, using a DeadEnd™ Fluorometric TUNEL System (Promega, Madison, Wis.), was performed following the manufacturer's protocols. Cell nuclei that were fluorescently stained with green were defined as TUNEL-positive nuclei. TUNEL-positive nuclei were monitored by using a fluorescence microscope (Nikon, Tokyo, Japan). The cell nuclei were stained with 4, 6-diaminidino-2-phenyl-indole (DAPI) Vectashield (Vector Laboratories, Inc., Burlingame, Calif.). TUNEL-positive cells in three slides of images taken at 30× magnification were counted to quantify apoptosis. CD-31 Antibody Staining. In order to observe the vasculature, the sections were incubated with a 1:250 dilution of CD31 primary antibody (Abcam, Cambridge, Mass.) at 4° C. overnight followed by incubation with FITC-labeled secondary antibody (1:200, Santa Cruz, Calif.) for 1 h at room temperature. The sections were also stained by DAPI and covered with a coverslip. The sections were observed using a Nikon light microscope (Nikon Corp., Tokyo, Japan).

Paraffin-embedded tumor sections were sequentially stained with Alexa Fluor® 647 for α-SMA (alpha smooth muscle Actin) immunofluorescence staining and FITC for TUNEL Assay. For immunofluorescence on α-SMA, the slides were deparaffinized through xylene and a graded alcohol series. After antigen was retrieved in antigen retrieval buffer (Tris-EDTA buffer, pH 9.0), all the sections were blocked by 1% bovine albumin (Sigma, USA) for 1 h at room temperature before they were incubated with a primary polycolonal rabbit anti-α-SMA antibody (Abcam, Cambridge, Mass., USA) at 1:100 dilution overnight at 4° C. Immunocomplexes were visualized with the corresponding Alexa Fluor® 647-labeled goat anti rat secondary antibody at a 1:100 dilution for 1 h at room temperature in the dark. Slides were then rinsed with PBS, prefixed with 4% formaldehyde. Apoptosis in situ was then detected by TdT-dependent dUTP-biotin nick end labeling (TUNEL) assay using apoptosis detection kit (Promega, Madison, Wis.) according to manufacturer's instructions. Slides were then rinsed with PBS and coverslipped with Vectashield with DAPI (Vector laboratories, Burlingame, Calif.). All staining was evaluated and digital images were acquired by Eclipse Ti-U inverted microscope (Nikon Corp., Tokyo, Japan)×20 magnification and quantitatively analyzed on Image J (National Institutes of Health).

### Example 1

### Loading

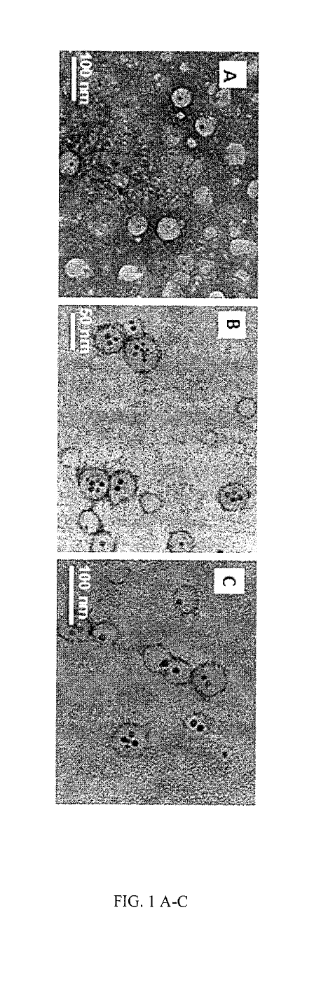

DOPA-CDDP cores, 12 nm in diameter, were loaded into MBA-PEG-PLGA NP with high efficiency (FIG. 1B-D). Twelve wt % of drug loading was achieved with an encapsulation efficiency of 82% (FIG. 2A). RAPA was also encapsulated alongside DOPA-CDDP cores (FIG. 2C). Although RAPA was encapsulated into the polymer matrix of PLGA NP via hydrophobic interactions, encapsulation was greatly limited by compatibility between the drugs and hydrophobic block of copolymers. When the feed ratio of RAPA to PLGA was 5 wt %, the encapsulation efficiency of RAPA was only 23%, correlating to a drug loading of only 1.15 wt % (FIG. 2 C). However, the presence of 4.5 wt % DOPA-CDDP core brings the EE and LE up to 80% and 4 wt %, respectively. The presence of DOPA-CDDP cores enhanced the loading of RAPA by 3.48-fold.

Transmission electron microscopy (TEM) showed spherical NP morphology with a diameter of approximately 40-50 nm, smaller than determined by DLS (FIG. 1 B-D and FIGS. 2 B and D). The number of DOPA-CDDP cores per NP can be controlled by adjusting the feed ratio between DOPA-CDDP cores and MBA-PEG-PLGA. The morphology and encapsulation of DOPA-CDDP cores were not affected by RAPA (FIGS. 2 E and F).

### Example 2

### Release

The release of both drugs at a predetermined, optimized rate and molar ratio is critical for effective combination therapy. These parameters must be fine-tuned to take full advantage of the synergistic action of these drugs. We measured the amount of CDDP and RAPA released from NP by ICP-MS and HPLC (FIG. 3A) Similar, sustained release rates were seen for both CDDP and RAPA. The release rate of each drug was independent from the other. Cellular uptake of MBA-PEG-PLGA NP was measured to confirm MBA-mediated targeting. Anisamide enhanced NP uptake by approximately 2-fold as shown by ICP-MS (FIG. 3B).

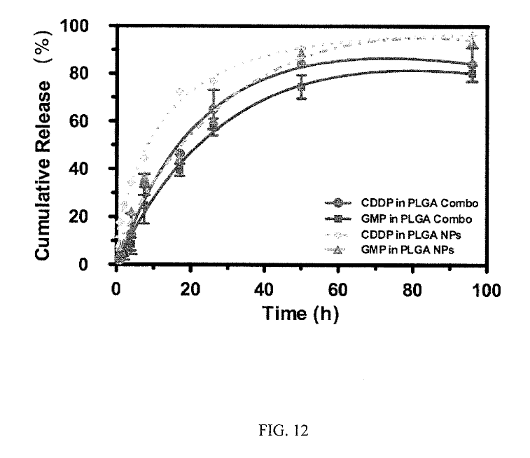

The in vitro release kinetics of CDDP and GMP from MBA-PEG-PLGA NPs were investigated by dialysis in pH 7.4 PBS and 37° C. for 96 hours to mimic physiological conditions. Release profiles of MBA-PEG-PLGA NPs loaded with only DOPA-GMP core, only DOPA-CDDP core or co-loaded with DOPA-GMP core and DOPA-CDDP core were evaluated. Platinum released from particles was measured by ICP-MS. ³H-labeled CMP (cytidine monophosphate) was used as a marker for the measurement of GMP release as previously mentioned. A known burst release phenomenon is often observed with hydrophilic drugs. For example, CDDP incorporated into PLGA15K-PEG5000 NPs showed a release fraction of approximately 50% after an initial 4 hours. Gemcitabine encapsulated PLGA NPs showed 60% liberated drug in less than 6 h. In our studies, there was only negligible burst release for our formulations when the drugs were encapsulated in DOPA-coated cores (FIG. 12).

Grouped T tests showed no significant difference between the release kinetics of the two drugs in combination (p=0.784), consistent with the expected ratiometric release profile. The subtle difference in observed release rate may be due to different composition of DOPA-CDDP and DOPA-GMP cores. This difference will likely become insignificant because of the release rate from MBA-PEG-PLGA NPs, the key rate-limiting step in the procedure. Another interesting observation was that drugs seem to be released more rapidly from their single formulation, also seen with RAPA/CDDP MBA-PEG-PLGA NPs combination. This may result from the differences in number of cores per particle or the interaction between cores of different drugs. It is concluded that release kinetics of CDDP and GMP from MBA-PEG-PLGA NPs also exhibited a ratiometric relationship.

### Example 3

### Synergy

We evaluated the degree of synergy between RAPA and CDDP in terms of cytotoxicity toward A375-luc cells in culture. CDDP+RAPA free drug combination with a CDDP:RAPA molar ratio of 5.5 to 1 resulted in a much lower IC₅₀ of 0.82 μM (CDDP concentration) compared to the IC₅₀ of free CDDP and RAPA (10 μM and 16 μM, respectively) (FIG. 3C). In contrast, (CDDP+RAPA) NP with a CDDP:RAPA molar ratio of 5.5 to 1 resulted in a even lower IC₅₀ of 0.30 μM (CDDP concentration) compared to the IC₅₀ of CDDP NP and RAPA NP (2.1 μM and 1.2 μM, respectively) (FIG. 3D). A combination index (CI) was determined using the Chou-Talalay isobologram equation.^{2,4} The CI at the IC₅₀ for combined free drug was 0.36, indicating synergy. The cytotoxicity of combined CDDP and RAPA was enhanced by NP delivery over free drug combination.

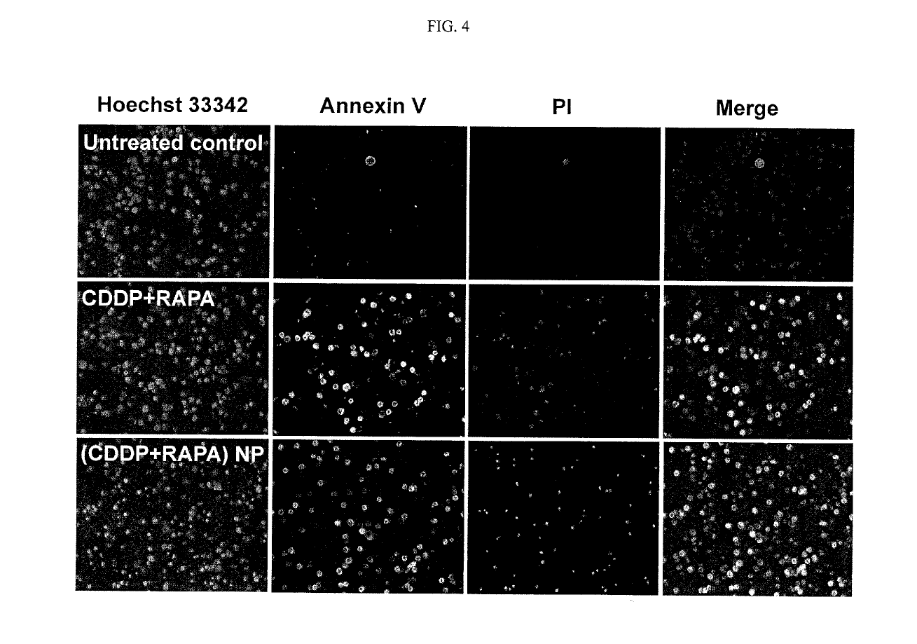

A375-luc cell apoptosis was measured after combined CDDP and RAPA treatment (at the IC₅₀ dose for CDDP) by using FITC-Annexin V/PI staining (FIG. 3E, FIG. 4 and FIG. 19-20). FIG. 3E presents the number of apoptotic cells detected by flow cytometry. Flow cytometry results were consistent with microscopy images (FIG. 4). RAPA sensitized A375-luc melanoma cells to CDDP. mTOR inhibitors may sensitize tumor cells to CDDP by blocking the up-regulation of p21 and inducing apoptosis as a result.

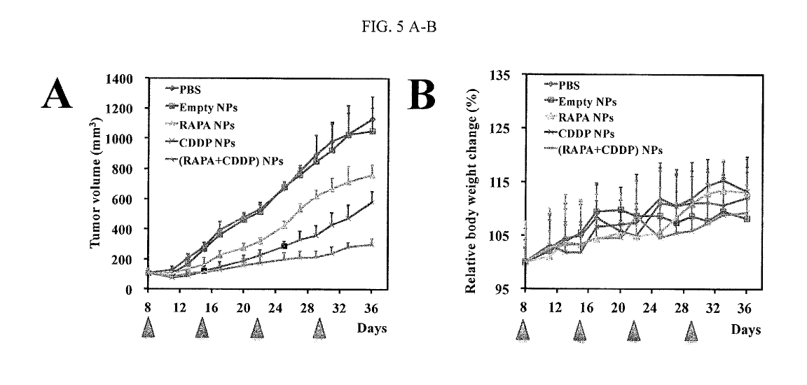

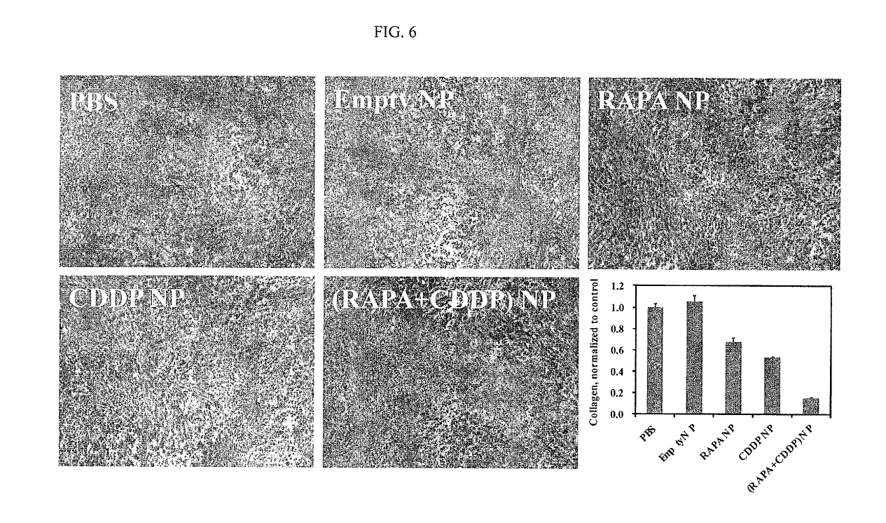

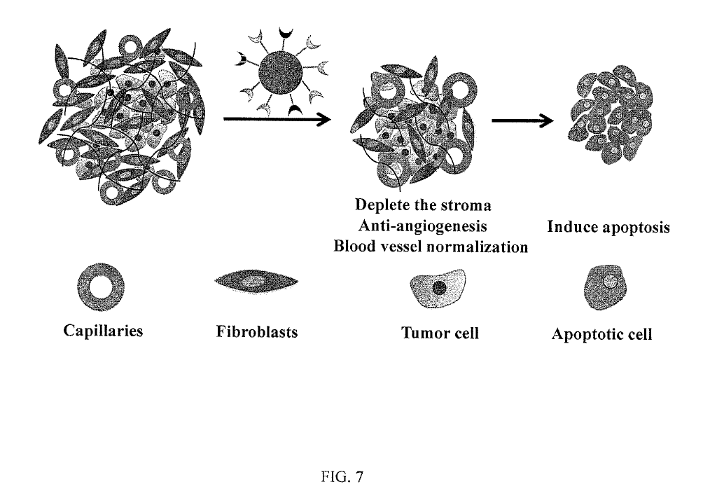

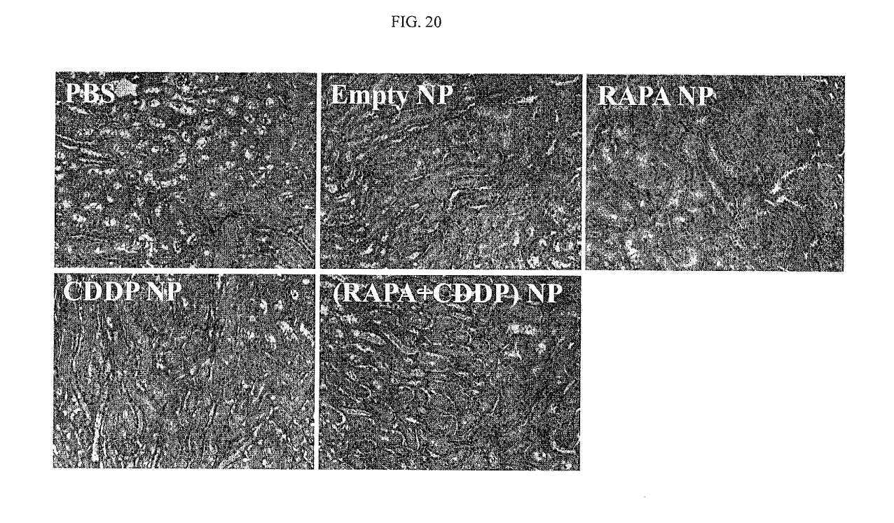

We tested the anti-cancer activity of (RAPA+CDDP) NP. Both RAPA NP and CDDP NP alone had minimal effect (FIG. 5A). (RAPA+CDDP) NP exhibited significant anticancer activity without reducing body weight of treated animals (FIG. 5). H&E staining confirmed the absence of observable nephrotoxicity (FIG. 20), supporting the likelihood that (RAPA+CDDP) NPs are both effective and safe. We then studied how treatment affected the tumor microenvironment. First, collagen, an extracellular matrix component, was stained with Masson Trichrome. CDDP NP and (RAPA+CDDP) NP treatment significantly reduced the amount of collagen (FIG. 6), possibly facilitating the perfusion of NP into the tumor and enhancing therapeutic effects (FIG. 7).

In sum, there are synergistic effects of RAPA and CDDP on viability of cultured A375M cells using (CDDP+RAPA) NPs (4.5 wt %/2.2 wt %) with a CDDP:RAPA molar ratio of about 5.5:1. Free CDDP and RAPA alone (FIG. 4B) each had an IC₅₀ of 10 μM and 16 μM, respectively. In combination, CDDP and RAPA had a much lower IC₅₀ of 0.82 μM (the concentration of CDDP). The Chou-Talalay combination index (CI) was 0.36 for free drug in combination, indicating synergism. Use of targeted NPs significantly enhanced the effect of combined CDDP and Rapamycin on cell viability (FIG. 4). Empty NPs had no effect, even at concentrations up to 10 mg/ml. The IC₅₀ of CDDP-alone NPs and RAPA-alone NPs were 2.1 μM and 1.2 μM, respectively, a five and thirteen-fold decrease compared to free drug. The IC₅₀ was approximately 0.3 μM (concentration of CDDP) for CDDP+RAPA NPs and the CI at the IC₅₀ was 0.50. These results confirm that any synergistic effects between CDDP and RAPA are maintained when combined in a single PLGA NP.

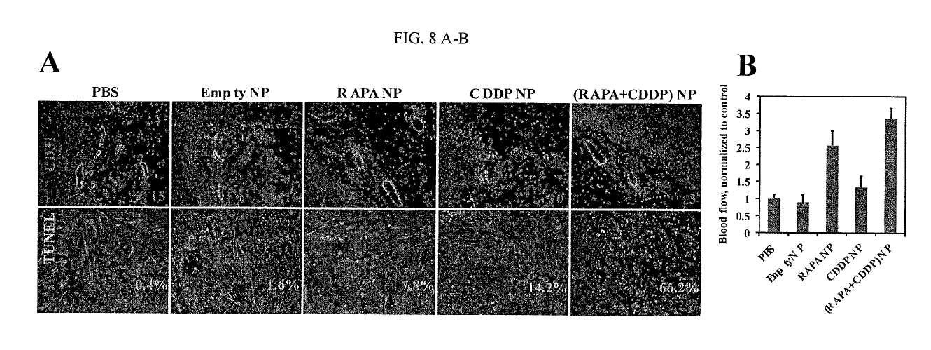

We further studied the effect of treatment on tumor fibroblasts by staining for α-smooth muscle actin (SMA) and apoptosis by TUNEL (FIG. 8A). CDDP NP and (RAPA+CDDP) NP induced apoptosis in tumor cells and also reduced the fibroblast population, probably resulting in lower levels of collagen. Five randomly selected microscopic fields contained 0.4% TUNEL-positive cells on average in the control group, 7.8% in the RAPA-treated group, 14.2% in the CDDP-treated group, and 66.2% in the combined therapy group. 25.1% of control group cells were SMA⁺. (RAPA+CDDP) NP almost completely depleted these cells with only 2.2% SMA after (RAPA+CDDP) NP treatment. (RAPA+CDDP) NP substantially changed the tumor microenvironment through depletion of cancer associated fibroblasts and reduction of collagen expression. Thus, (RAPA+CDDP) NP not just targeted to tumor cells, but also fibroblasts in the microenvironment.

MTT assays were carried out to test anticancer effects in cultured UMUC-3 cells. After 48 h of treatment, free CDDP, free CDDP and GMP combo, single MBA-PEG-PLGA NPs loaded with DOPA-GMP core and DOPA-CDDP core separately, MBA-PEG-PLGA NPs co-loaded with DOPA-GMP core and DOPA-CDDP core caused a dose dependent reduction in the number of viable UMUC-3 cells, although free GMP reached a plateau at higher concentration. MBA-PEG-PLGA NPs containing anisamide likely enter cells through sigma receptor-mediated endocytosis, while free GMP is metabolized to gemcitabine before entering cells through nucleoside transporters. The saturation of nucleoside transporters may explain the dose-independent response of free GMP at higher concentration. Although, there is a subtle difference between the IC₅₀ of free CDDP and MBA-PEG-PLGA NPs loaded with DOPA-CDDP cores, NP delivery resulted in a much lower IC₅₀ of 17.8 μM compared with free drug (IC₅₀ of 34.8 μM The Chou-Talalay combination index (CI) was determined to be 0.41 at IC₅₀, confirming synergistic combination therapy and supporting further tumor inhibition studies in vivo.

### Example 4

### In-Vivo

RAPA has known anti-angiogenic properties. Blood vessels were stained with an anti CD31 antibody (FIG. 8A), showing a marked decrease in the number of vessels while mice treated with CDDP alone were unaffected Inhibition of angiogenesis was more pronounced in the combined therapy group. Blood vessel normalization was also observed, consistent with previous reports in breast cancers. NVP-BEZ235, a dual inhibitor of phosphoinositide-3-kinase (PI3K) and mTOR, also had a normalization effect on tumor vasculature. Normalization may enhance transvascular flux and improve delivery of NP to the tumor. To characterize blood flow, vessel area was quantified by ImageJ and normalized to data from the control group. (RAPA+CDDP) NP increased approximately 3-fold vessel area over the PBS control group (FIG. 8).

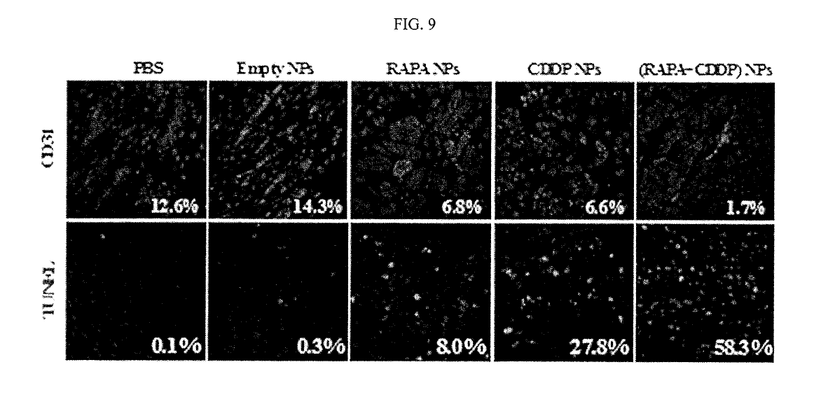

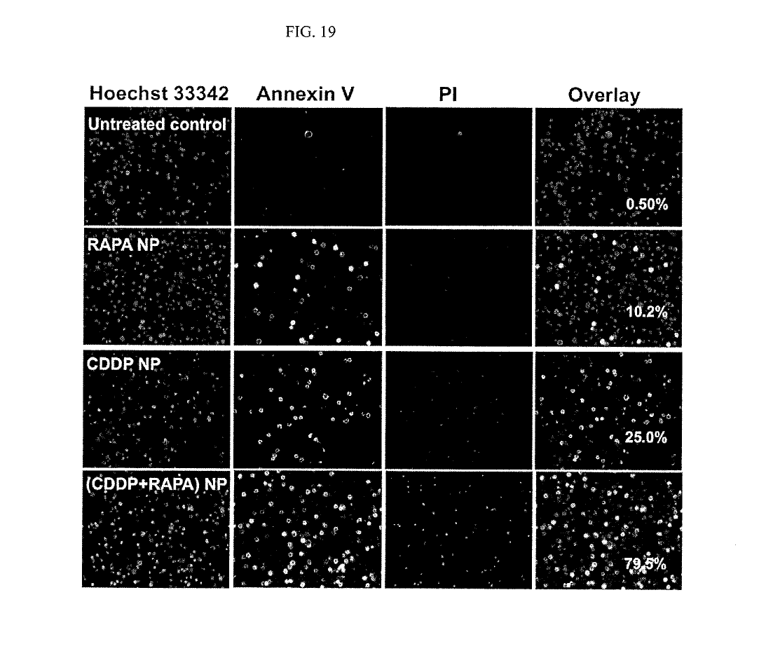

Apoptosis of A375M cells treated with CDDP and RAPA was assayed using FITC-Annexin V/PI staining using an IC50 dose of CDDP (FIGS. 4 and 19). The percentage of apoptotic cells was measured by flow cytometry and was consistent with microscopy results (FIG. 4). It is clear that RAPA sensitized A375M melanoma cells to CDDP. The anti-cancer efficacy of (RAPA+CDDP) NPs was tested in tumor bearing mice. Both the RAPA NPs and CDDP NPs alone were minimally effective while RAPA+CDDP NPs most effectively reduced tumor volume. The NPs had no effect onbody weight of treated animals (FIG. 9) and there was no evidence of nephrotoxicity seen in H&E stains of kidney tissue from treated mice (FIG. 20). RAPA exhibits known anti-angiogenic properties. To investigate the effect of RAPA, immunohistologic staining of tumor blood vessel endothelial cells was conducted with anti CD31 antibody. RAPA-treatment decreased in the density of tumor blood vessels while CDDP had no effect. Combined therapy had a more pronounced effect than RAPA alone.

We compared the effect of RAPA alone to RAPA+CDDP combination therapy on tumor cell apoptosis by TUNEL (FIG. 5C). In five randomly selected microscopic fields, the average percentage of TUNEL-positive cells was 0.1% in the control group, 18% in the RAPA-treated group, 28% in the CDDP-treated group, and 58% in the combined therapy group, suggesting that RAPA inhibited tumor growth by inhibiting angiogenesis and through synergistic effects with CDDP.

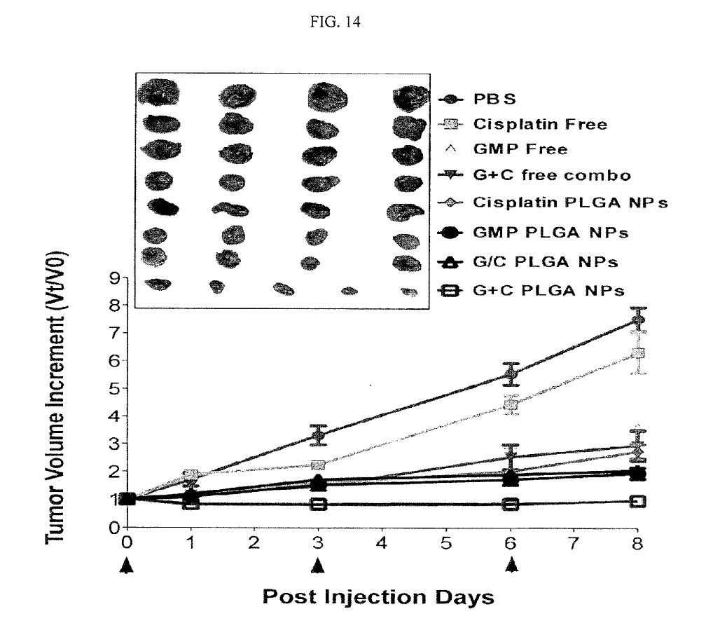

Studies were performed to test the anti-cancer efficacy of MBA-PEG-PLGA NPs co-loaded with DOPA-GMP and DOPA-CDDP cores in tumor bearing mice. UMUC-3 cells were subcutaneously co-inoculated along with NIH 3T3 fibroblasts in matrigel. Tumors developed for five days until volumes reached 150-200 mm3 Mice were then treated with a total of 3 injections of combined 8 mg/kg GMP and 1.6 mg/kg CDDP in PLGA (FIG. 14A). NPs loaded with a single drug were less effective, while combination therapy resulted in potent tumor inhibition.

The efficacy of delivered drugs was considerably enhanced by MBA-PEG-PLGA NP based delivery. DOPA-CDDP cores and RAPA acted synergistically to induce apoptosis of cancer cells both in vitro and in vivo. We also co-encapsulated DOPA-GMP and DOPA-CDDP cores into MBA-PEG-PLGA NPs with a controlled ratio and high efficiency. These NPs showed a controlled release profile for both GMP and CDDP. Preliminary data show that these NPs exhibit synergistic effects against bladder cancer in vitro and in vivo. Additionally, hydrophobic and lipid-coated drugs can be efficiently loaded together, creating the opportunity to combine bioactive compounds having diverse physiochemical properties.

### Examples 5-15

### Chemicals and Materials

Gemcitabine monophosphate disodium salt (GMP, purity ≧97%) was obtained. Cisplatin was purchased from Sigma-Aldrich (Dorset, UK). Dioleoyl phosphatidic acid (DOPA) was obtained from Avanti Polar Lipids, Inc. (Alabaster, Ala.). mPEG₃₀₀₀-NH₂.HCl and tBOC-PEG₃₅₀₀-NH₂.TFA were purchased from JenKem Technology USA Inc. (Allen, Tex.). Acid terminated PLGA was ordered from DURECT Corporation (Cupertino, Calif.). N,N-diisopropylethylamine (DIPEA), 1-ethyl-3-(3-(dimethylamino)-propyl)carbodiimide (EDC), N-hydroxysuccinimide (NHS), dichloromethane, Triton™ X-100, Igepal® CO-520, p-Anisic acid, silver nitrate and cyclohexane were purchased from Sigma-Aldrich (St Louis, Mo.) without further purification.

### Cell Culture:

The mouse embryonic fibroblast cell line (NIH 3T3) was obtained from UNC Tissue Culture Facility. The human bladder transitional cell line (UMUC3) was generously provided by Dr. William Kim (University of North Carolina at Chapel Hill, N.C.). These two cell lines were cultured in Dulbecco's Modified Eagle's Media (DMEM) (Invitrogen, Carlsbad, Calif.), supplemented with streptomycin (100 μg/mL) (Invitrogen), penicillin (100 U/mL), and 10% Bovine calf serum (Hyclone, Logan, Utah) or 10% fetal bovine serum (Sigma, St. Louis, Mo.) respectively. Cells were cultured in a humidified incubator at 37° C. with 5% CO₂.

### Experimental Animals:

Female athymic nude mice used in all the studies weighed between 28-22 g and were 6-8 weeks of age.

### Synthesis of PLGA-PEG-MBA and PLGA-mPEG:

For the synthesis of PLGA-PEG-MBA, tBoc-PEG₃₀₀₀-NH₂.HCl (1 eq), anisic acid (8 eq) and DIPEA (4 eq) were dissolved in DCM and added with DIC (8 eq) to react for 26 h to obtain MBA-PEG-Boc. After purification and structure confirmation by NMR, Boc protecting group was removed using a TFA/DCM (1:2, v/v) mixture to achieve MBA-PEG-NH₂.TFA. Afterwards, MBA-PEG-NH₂.TFA was conjugated to PLGA (15 kDa, 0.1 mmol) in the presence of DIPEA and DIC for 26 h and purified. PLGA-PEG-MBA structure was confirmed by NMR. In the synthesis of PLGA-mPEG, mPEG-NH₂.TFA (3000, 0.126 mmol), PLGA (15 kDa, 0.1 mmol) and DIPEA (0.5 mmol) were dissolved in 6 ml DCM and reacted with DIC (1.0 mmol) for 24 h.

### Preparation of CP Cores:

CP cores were prepared as previously mentioned with a little adjustment. (S. Guo, Y. Wang, L. Miao, Z. Xu, C. M. Lin, Y. Zhang, L. Huang, ACS Nano 2013, 7, 9896; S. Guo, L. Miao, Y. Wang, L. Huang, J. Control. Release 2014, 174, 137). First, 300 μL of 200 mM cis-[Pt(NH₃)₂(H₂O)₂](NO₃)₂ was dispersed in a mixed solution of cyclohexane/triton-X100/hexanol (75:15:10, V:V:V) and cyclohexane/Igepal CO-520 (71:29, V:V) to form a well-dispersed reversed micro-emulsion. Another reversed micro-emulsion containing KCl was prepared by adding 300 μL of 800 mM KCl aqueous solution to a separate oil phase. Then, 500 μL of DOPA (20 mM) was added to the cisplatin precursor phase and the mixture was stirred. Twenty minutes later, the two emulsions were mixed and reacted for another 20 min. Forty mL of ethanol was then added to break the micro-emulsion and the mixture was centrifuged at 10,000 g for at least 15 min. The pellets were washed with ethanol 2 more times to ensure the complete removal of the surfactants and cyclohexane, and then re-dispersed in 2.0 mL of chloroform for storage.

### Preparation of GMP Cores:

GMP cores were synthesized according to our previous work with a little adjustment. (Y. Zhang, L. Peng, R. J. Mumper, L. Huang, Biomaterials 2013, 34, 8459). Briefly, 100 μL of 60 mM GMP was mixed with 500 μL of 25 mM Na₂HPO₄ and then dispersed in 20 mL of oil phase containing Igepal CO-520/cyclohexane (29:71, V:V). The other emulsion was prepared by adding 600 μL of 2.5 M CaCl₂ into a separate oil phase. Six-hundred mL of 20 mM DOPA was added to the phosphate phase before mixing of the two separate emulsions. Another 400 μL of 20 mM DOPA was added to the combined emulsion 5 min after mixture. The emulsion was stirred for another 20 min and then 40 mL of ethanol was added. Next, the mixture was centrifuged at 10,000 g for 15 min to remove the surfactants and cyclohexane. After being washed with ethanol 2-3 times, the pellets were re-dispersed in 2.0 ml of chloroform for storage.

Preparation of PLGA/PLGA-PEG/PLGA-PEG-MBA (4:4:2) NP (PLGA NP) Loaded with Cores:

Drug encapsulated cores were loaded into PLGA NP using a single step solvent dispersion method as previously described with little adjustment. (S. Guo, C. M. Lin, Z. Xu, L. Miao, Y. Wang, L. Huang, ACS Nano., DOI: 10.1021/nn5010815). Briefly, 2 mg of polymers and the cores were dissolved in 200 μl of THF and added dropwise into 2 ml of water with continuous stirring at room temperature. Then, the NP suspension was stirred uncovered for 6 h at room temperature in order to remove the residual THF. The resulting NP were further purified by ultrafiltration (3000×g, 15 min, Amicon Ultra, Ultracel membrane with 50,000 NMWL, Millipore, Billerica, Mass.). The obtained PLGA NP were then re-suspended, washed twice with water, centrifuged at 14,000 rpm for 20 min to further remove free lipids and micelles. And then re-suspended again and centrifuged at 800 rpm to remove nanocore aggregations.

### Characterization of PLGA NP:

DL and EE of cisplatin were measured using Inductively Coupled Plasma-Mass Spectroscopy (ICP-MS, NexION™ 300, Perkin Elmer Inc); LE and EE of GMP were both measured by Ultraviolet-Visible Spectroscopy (UV, DU®800, Beckman Coulter) and ³H labeled cytidine 5′ monophosphate (CMP) [5-³H] disodium salt (Moravek Bio Inc, 1 mCi/mL) incorporation using a Liquid Scintillation Analyzer (TRI-CARB 2900 TR, Packard Bioscience Co). The size distribution of particles was determined using a Malvern ZetaSizer Nano series (Westborough, Mass.). TEM images of NP were obtained using a JEOL 100CX II TEM (JEOL, Japan). For NP imaging, the NP were negatively stained with 2% uranyl acetate. The composition of PLGA combo NP was studied using Electron Dispersive Spectroscopy (EDS) (Oxford instruments, INCA PentaFET-×3) and X-ray photoelectron spectroscopy (XPS) (Kratos Axis Ultra DLD X-ray Photoelectron Spectrometer).

### Cellular Uptake Study in UMUC3 Cell Lines:

UMUC3 cells were seeded into a 12-well plate (1.5×10⁵ cells/well) containing 1 ml of media. Twenty-four hours later, 1 ml of the free drug combination, targeted PLGA Combo NP, targeted PLGA Sepa NP, 20%-targeted PLGA Combo NP or non targeted PLGA Combo at a concentration of 20 μM GMP and 3.8 μM cisplatin were incubated with cells in a serum-free medium. Four hours later, cells were treated with RIPA buffer (Sigma-Aldrich). The concentration of cisplatin was measured using ICP-MS and GMP was measured as ³H-CMP using a scintillation counter as previously mentioned.

In Vitro Release and Intracellular Release of Cisplatin and GMP from PLGA NP:

The dialysis technique was employed to study the in vitro release of GMP and cisplatin from PLGA in phosphate buffered saline (PBS) (pH 7.4 or pH 5.6) at 37° C. Five hundred μL of PLGA NP loaded with 100 μg/mL of GMP and cisplatin separately or co-loaded with 100 μg/mL of GMP and cisplatin at a ratio of 5.33:1 were added into the dialysis tube with a molecular weight cut off of 3000 Da and dialyzed against 15 mL of PBS (pH 7.4 or pH 5.6) in a thermo-controlled shaker with a stirring speed of 200 rpm at 37° C. for 96 h. In the preparation of GMP cores, a trace amount of radioactive ³H-CMP was mixed with GMP to serve as a marker for the entrapped GMP. At each predetermined time point, 400 μL samples were taken and replaced with fresh media. Platinum and GMP concentrations were then determined by ICP-MS and scintillation analyzer respectively at specified times. All experiments were performed in triplicate and the data were reported as mean±SD of the three individual experiments. Measuring of intracellular release of free drugs from the nanoparticles was carried out according to a previous protocol. (S. Guo, Y. Wang, L. Miao, Z. Xu, C. M. Lin, Y. Zhang, L. Huang, ACS Nano 2013, 7, 9896). Briefly, a 12-well plate of UMUC3 cells was prepared as mentioned in the uptake study and incubated with 20 μM of GMP and 3.8 μM of cisplatin encapsulated into PLGA Combo NP. After 1, 4, and 16 hours, the cells were treated with 50 μL of RIPA buffer (Sigma-Aldrich) at 4° C. for 10 min and the cell lysate was centrifuged at 14,000 rpm for 20 min at 4° C. to separate nanoparticle and cell lysate from free drugs. Free drugs and nanoparticles were measured using ICP-MS and ³H-labeled scintillation. All experiments were performed in four replicates and the data reported as mean±SD.

### In Vitro Cell Viability on UMUC3 Cells and Analysis of Synergistic Effects of Drug Combinations:

MTT (3-[4,5-dimethylthiazol-2-yl]-2,5-diphenyltetrazolium bromide) assay was conducted to detect in vitro viability of free GMP, cisplatin and their combinations as well as PLGA GMP NP, PLGA cisplatin NP and PLGA Combo NP. In Brief, cells were seeded in 96-well plates with a density of 3,000 cells per well 24 h prior to drug treatment. On the second day, cells were treated with free drugs or the drug combination at a series of dilutions with various molar ratios. Forty-eight h post treatment, 20 μL of MTT (5 mg/mL) reagent was added for another 4 h at 37° C. The medium was then discarded and the formed formazan salt was dissolved in 150 μL of DMSO. The absorbance in each well was read at the wavelength of 570 nm using a multidetection microplate reader (Plate CHAMELEON™ V-Hidex). Each concentration was tested in five wells and data presented as mean±SD. The mean drug concentration required for 50% growth inhibition (IC₅₀) was calculated using CompuSyn software (Version 1.0, Combo-Syn Inc., U.S.) with the median effect equation: Fa=[1+(IC₅₀/D)^{m}]⁻¹, where, m is the Hill slope, D is drug concentration and Fa is the fraction of affected cells.

Combination Index (CI) Analysis of free drug combination based on the Chou and Talalay method (T. C. Chou, P. Talalay, Adv. Enzyme Regul. 1984, 22, 27) was conducted using CompuSyn software. Briefly, for each level of Fa, the CI values of cisplatin and GMP combinations were calculated according to the following equation: CI=(D)₁/(Dₓ)₁+(D)₂/(Dₓ)₂, where (Dx)₁ and (Dx)₂ are the concentrations of the drugs alone resulting in Fa×100% growth inhibition, while (D)₁ and (D)₂ are the concentrations of each drug in the combination leading to Fa×100% growth inhibition. CI values of the drug combinations were drawn as a function of Fa. CI values more than 1 or less than 1 indicate antagonism or synergism of drug combinations, respectively. Notably, CI values between Fa 0.2 to 0.8 are considered validate. (Y. Han, Z. He, A. Schulz, T. K. Bronich, R. Jordan, R. Luxenhofer, A. V. Kabanov, Mol. Pharm. 2012, 9, 2302).

### Tumor Accumulation of GMP and Cisplatin in Stroma-Rich Xenograft Bladder Tumor Model:

A stroma-rich subcutaneous xenograft bladder tumor model was established previously in our lab. (J. Zhang, L. Miao, S. Guo, Y. Zhang, L. Zhang, A. Satterlee, W. Y. Kim, L. Huang, J. Control. Release, DOI 10.1016/j.jconre1.2014.03.016). Briefly, UMUC3 (5×10⁶) and NIH 3T3 cells (2×10⁶) in 100 μL of PBS were subcutaneously co-injected into the right flank of mice along with Matrigel (BD Biosciences, CA) at a ratio of 3:1 (v/v). When the tumor reached 100-150 mm² in size, animals were randomly divided into three groups (n=8) and intravenously injected with free GMP and cisplatin (Combo Free), PLGA Combo NP and PLGA Sepa NP at a dose of 12 mg/kg GMP and 1.9 mg/kg cisplatin respectively. Trace fraction of ³H-CMP was added to the GMP related groups for the measurement of tumor accumulation of GMP. Four mice from each group were sacrificed at each predestined time point and tumor tissues were collected. Tumor uptake of GMP and cisplatin was expressed as the percentage of the injected dose per gram tumor. For measurement of GMP, 10-20 mg of tumor tissue was immediately mixed with 10×NCS® II Tissue Solubilizer (Amersham Biosciences, Inc) and digested at 60° C. overnight. Three hundred μL of hydrogen peroxide (30% in water, Fisher) was then added to the samples and vortexed to bleach the color. The sample was then mixed with 4 mL of scintillation cocktail (Fisher Inc). The radioactivity of ³H in the tumor samples was counted using a liquid scintillation analyzer (TRI-CARB 2900 TR, Packard Bioscience Co.). For the measurement of cisplatin, 25-35 mg of tumor tissue was digested with 400 μL 60% nitric acid (Acros Organic) at 70° C. overnight and the amount of platinum was measured by ICP-MS.

### Biodistribution of Dual Drug in Major Organs:

Mice were administered a single dose of Combo Free, PLGA Sepa NP and PLGA Combo NP respectively at a dose of 1.9 mg/kg cisplatin and 12 mg/kg GMP. Each group contained four mice, which were sacrificed 10 h following injection. Tissue samples were digested as previously mentioned in the tumor accumulation study. Cisplatin was quanified via ICP-MS and GMP via scintillation counter.

### Anti-Tumor Efficacy in Stroma-Rich Xenografts:

When the inoculated tumor reached 100-150 mm² in size, mice were randomized into eight groups (n=5) as follows: Untreated Control (PBS), free GMP (GMP free), free cisplatin (Cisplatin Free), combination of free GMP and cisplatin (Combo free), PLGA GMP NP, PLGA cisplatin NP, GMP and cisplatin PLGA NP mixtures (PLGA Sepa NP) as well as PLGA Combo NP. IV injections were performed every three days for a total of three injections with a GMP dose of 12 mg/kg and a cisplatin dose of 1.9 mg/kg. Tumor volume was measured every day. Body weight was also recorded. Mice were sacrificed two days after the last injection and tumor tissues were collected for further study.

### TUNEL Assay:

All the immunofluorescence detections mentioned in this manuscript on UMUC tumor bearing mice were prepared using paraffin embedded sections (prepared by the UNC Tissue Procurement Core). Slides were deparaffinized through xylene and a graded alcohol series and prefixed with 4% formaldehyde. Apoptosis was then detected by TdT-dependent dUTP-biotin nick end labeling (TUNEL) assay using an apoptosis detection kit (Promega, Madison, Wis.) following the manufacturer's instructions. Slides were then coverslipped using VECTASHIELD with DAPI (Vector laboratories, Burlingame, Calif.). All staining was evaluated and digital images were acquired by an Eclipse Ti-U inverted microscope (Nikon Corp., Tokyo, Japan) at 20× magnification. Five randomly selected microscopic fields were quantitatively analyzed using Image J (National Institutes of Health).

### PCNA Assay:

Proliferation of tumor cells after the aforementioned treatments was detected by antibody against proliferating cell nuclear antigen (PCNA) (1:200 dilution, Santa Cruz). (Y. Zhang, W. Y. Kim, L. Huang, Biomaterials 2013, 34, 3447) The paraffin embedded tissue sections were deparaffinized, antigen recovered, blocked and probed with PCNA antibody overnight at 4° C., and then detected using a mouse-specific HRP/DAB detection IHC kit as recommended by the manufacturer (Abcam, Cambridge, Mass.). Cell nuclei were counter-stained with hematoxylin. The percentage of proliferative cells was calculated by dividing the number of PCNA positive cells (shown as brown dots) by the number of total cells (blue nuclei stained by hematoxylin). Five representative microscopic fields were randomly selected in each treatment group for quantification.

### Platinum Adduct Staining:

The platinum-DNA adducts were detected using anti-cisplatin modified DNA antibodies [CP9/19] (Abcam, Cambridge, Mass.). (S. Guo, Y. Wang, L. Miao, Z. Xu, C. M. Lin, Y. Zhang, L. Huang, ACS Nano 2013, 7, 9896). The tumor sections were deparaffinized, antigen recovered, blocked with 1% BSA/PBS for 1 h at room temperature, incubated with a 1:250 dilution of anti-cisplatin modified DNA antibody [CP9/19] at 4° C. overnight, and then incubated with FITC-labeled goat anti-rat IgG antibody (1:200, Santa Cruz, Calif.). The sections were also counter-stained with VECTASHIELD mounting media with DAPI (Vector laboratories, Burlingame, Calif.). The tumor sections were observed and quantified using a Nikon light microscope (Nikon Corp., Tokyo, Japan).

### Western-Blot Analysis:

Two days after three daily IV injections, UMUC tumor bearing mice were sacrificed and tumor tissues were collected and lysed using radioimmunoprecipitation assay (RIPA) buffer (Sigma-Aldrich). The concentration of total protein in the tumor lysate was quantified using bicinchoninic acid (BCA) protein assay reagent following the manufacturer's instruction (Invitrogen). After dilution with 4× sample buffer containing reducing agent and heating at 95° C. for 5 min, forty μg of protein per lane was separated by 4-12% SDS-PAGE electrophoresis (Invitrogen). The proteins were then transferred to polyvinylidene difluoride (PVDF) membranes (Bio-Rad). The membranes were blocked with 5% skim milk for 1 h and incubated overnight at 4° C. with mouse monoclonal poly(ADP-ribose) polymerase-1 (PARP-1) antibodies, mouse monoclonal ERCC1, mouse monoclonal XPA (12F5). GAPDH antibody (1:4000 dilution; Santa Cruz biotechnology, Inc.) was used as the internal loading control. The membranes were washed three times and then incubated with a secondary antibody (1:4000 dilution; Santa Cruz biotechnology, Inc.) at room temperature for 1 h. Goat anti-mouse secondary antibody was used for PARP, XPA and ERCC-1 primary antibody. Goat anti-rabbit secondary antibody was used for GAPDH primary antibody. Finally, the membranes were washed four times and detected using the Pierce ECL Western Blotting Substrate according to the manufacturer's instructions (Thermo Fisher Scientific).

### Serum Biochemical Value Analysis and Hematology Assay:

After three injections, blood was collected and centrifuged at 4000 rpm for 5 min to obtain the serum. Blood urea nitrogen (BUN), creatinine, serum aspartate aminotransferase (AST) and alanine aminotransferase (ALT) levels, were assayed as indicators of renal and hepatic function. Whole blood was collected from healthy nude mice after three repeated treatments. Red blood cells (RBC), white blood cells (WBC), platelets (PLT), hemoglobin (HGB) and hematocrits (HCT) were counted for the detection of myelosuppression. Organs (heart, liver, spleen, lung, and kidney) were fixed and sectioned for H&E staining as to evaluate the organ-specific toxicity.

### Statistical Analysis:

Quantitative results were expressed as mean±SD. The analysis of variance was completed using student's t-test and one-way analysis of variance (ANOVA). A p value of p<0.05 was considered statistically significant.

### Example 5

### Preparation and Characterization of Single Drug Loaded PLGA NP

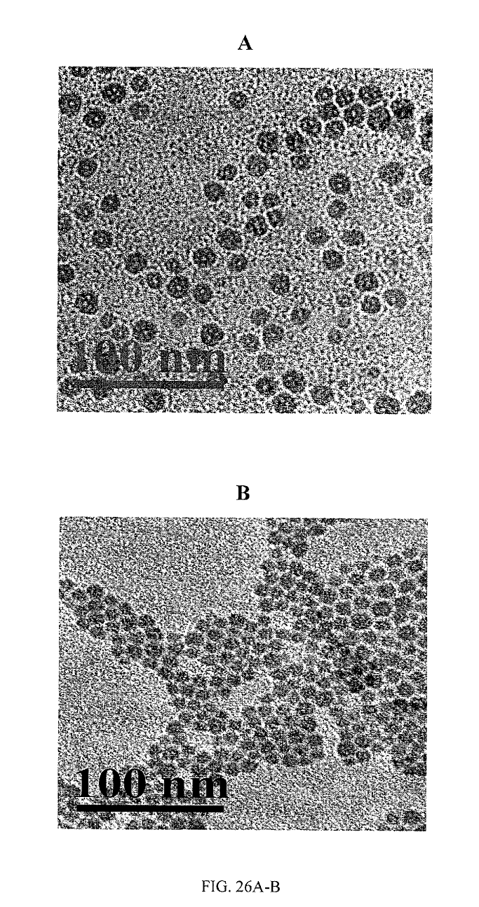

GMP cores and CP cores were prepared as previously mentioned (S. Guo, et al., ACS Nano 2013, 7, 9896; Y. Zhang, et al., Biomaterials 2013, 34, 3447) and characterized as 8-12 nm in diameter as determined by transmission electron microscopy (TEM) (FIG. 26). Encapsulation efficiency (EE) of GMP in the GMP core was 60.6±4.3% (n=5) as measured by absorbance of GMP at 273 nm CP cores were also prepared with an EE of 40.4±1.4% (n=5) as measured by inductively coupled plasma mass spectrometry (ICP-MS). Both GMP cores and CP cores could be well dispersed into organic solvent, such as tetrahydrofuran (THF). The above results show that hydrophilic GMP and cisplatin have been successfully loaded into hydrophobic cores respectively and these cores were ready to be further incorporated into PLGA NP.

High and comparable encapsulation efficiency of each component is a proposed prerequisite for controlled loading of several modalities in the same nanoparticle. Therefore, single drug loaded PLGA NP was characterized. PLGA NP were conjugated with polyethylene glycol (PEG) to prolong systemic circulation time and then self-assembled with PLGA and DOPA coated cores into PLGA NP via single step solvent displacement (FIG. 21).

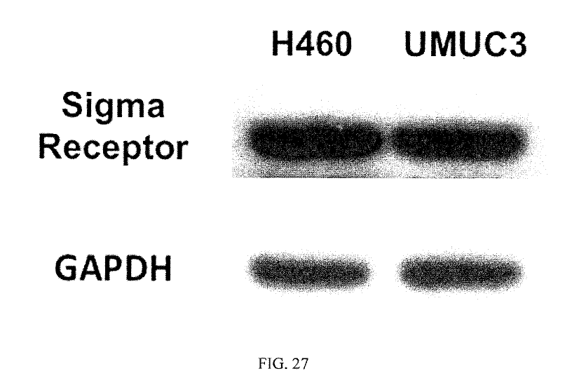

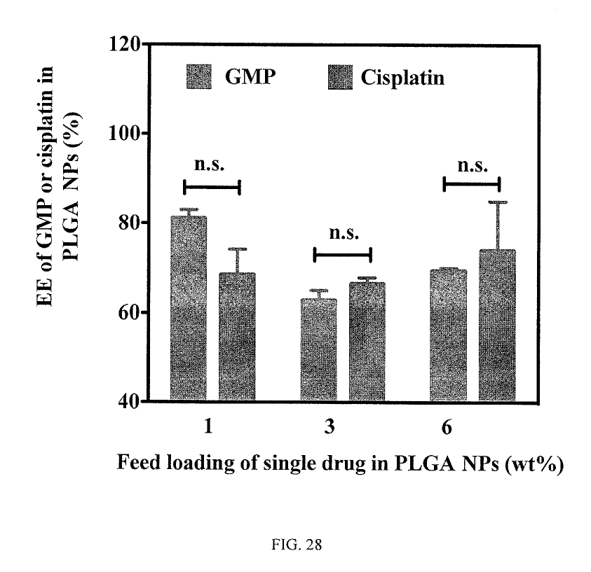

Polymer and drug containing cores were dissolved in THF, a water-miscible solvent, and poured drop wise into water. NP was formed instantaneously during this rapid solvent diffusion process. Anisamide, an agonist of the sigma receptor, was also introduced into PLGA NP as a ligand to enhance internalization in epithelium-derived cancer cells, which overexpress the sigma-receptor (FIG. 27). (a) J. L. Vivero-Escoto, K. M. Taylor-Pashow, R. C. Huxford, J. Della Rocca, C. Okoruwa, H. An, W. Lin, W. Lin, Small 2011, 7, 3519; b) O. Nakagawa, X. Ming, L. Huang, R. L. Juliano, J. Am. Chem. Soc. 2010, 132, 8848). Results in FIG. 28 indicate that both GMP and cisplatin in DOPA coated core structures can be encapsulated into PLGA NP separately with high EE (70.6±2.5% and 74.0±10.1%, respectively, n=5) at drug loading (DL) of up to approximately 5 wt %. As described herein, GMP and cisplatin have been engineered into PLGA NP using solvent displacement method.

This method is significantly more efficient than loading free gemcitabine and cisplatin into PLGA NP via the double emulsion method, whose maximum loading is only around 1 wt %. (a) K. Avgoustakis, A. Beletsi, Z. Panagi, P. Klepetsanis, A. G. Karydas, D. S. Ithakissios, J. Control. Release 2002, 79, 123; b) S. Aggarwal, S. Yadav, S. Gupta, J. Biomed. Nanotechnol. 2011, 7, 137). Notably, free cisplatin and GMP are quite polar and cannot be loaded into PLGA NP using the solvent displacement method; and thus, DOPA coated cores not only provide an approach to load different types of drugs, especially hydrophilic drugs, into PLGA NP using solvent displacement, but also facilitate hydrophilic drugs to be loaded into PLGA NP with higher DL and EE. More importantly, the EE for single free drugs in PLGA NP using this novel preparation method described herein can facilitate loading different drug moieties simultaneously into the same NP at similar EE but different dual-drug ratios, which is one indispensable parameter for ratiometric loading.

### Example 6

### Precise Ratiometric Control Over Dual-Drug Loading in Combo NP

Loading GMP cores and CP cores into PLGA NP provides a means to encapsulate two different drug-containing cores into a single NP in a ratiometric manner. The following studies further confirm that CP cores and GMP cores can be ratiometrically co-loaded into PLGA NP (Combo NP).

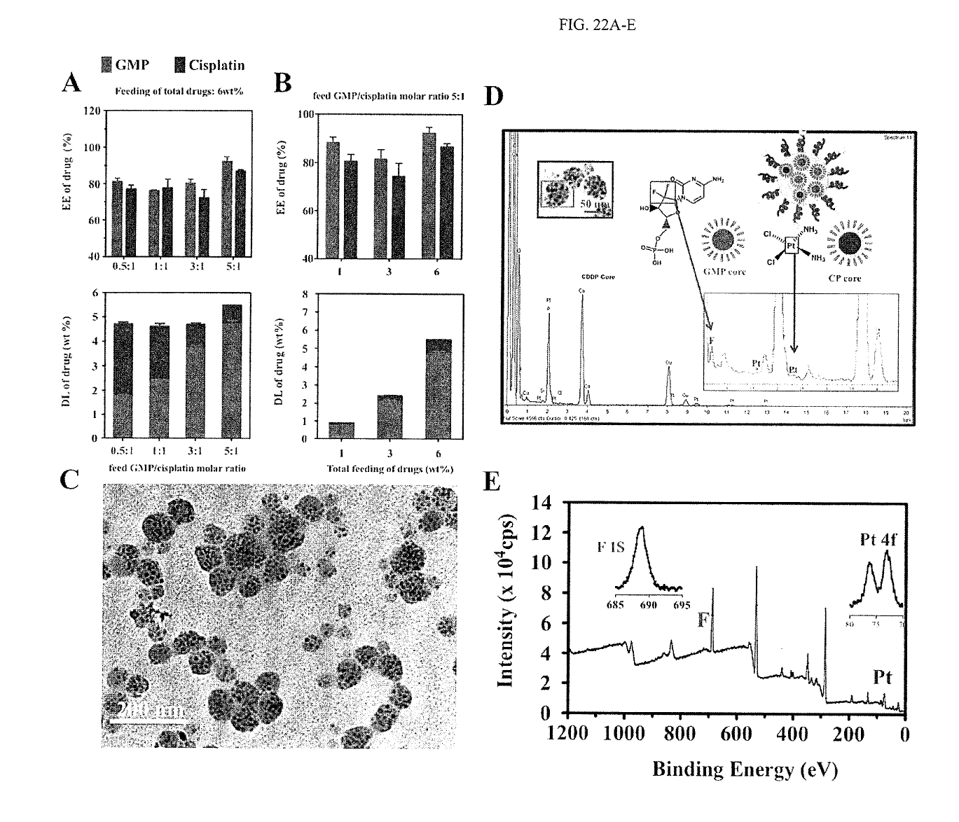

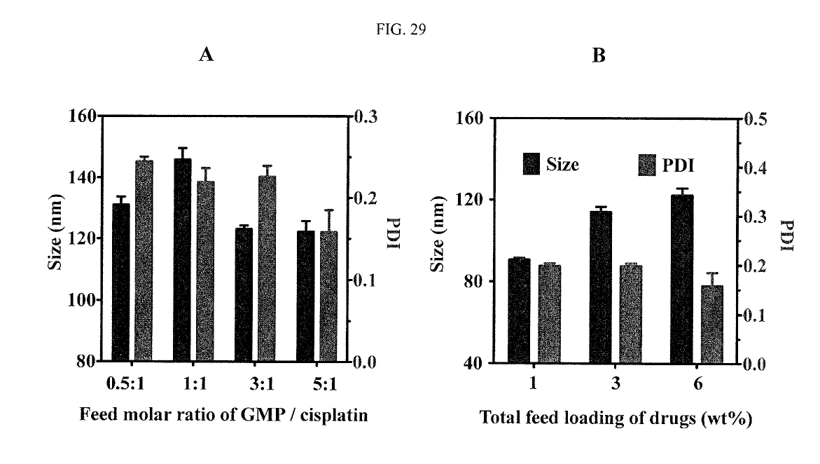

Firstly, total feed loading of GMP and cisplatin in Combo NP was fixed at 6 wt % while the feed molar ratio between GMP and cisplatin was altered from 0.5:1 to 5:1 (FIG. 22A). Results indicated that the measured molar ratio between the two drugs in Combo NP was almost the same as the feed molar ratio (0.52 vs 0.5; 0.97 vs 1; 3.3 vs 3, 5.3 vs 5) and the EE of both drugs, which all remained above 70% with subtle fluctuation, was almost identical as well. Next, the feed molar ratio of GMP to cisplatin was set at 5 (FIG. 22B). It was found that the measured molar ratio of GMP to cisplatin in Combo NP was approximately 5 when the total loading of the two drugs was below 6 wt %. Additionally, greater than 80% EE was achieved. In both experiments, particle size measured by dynamic light scattering (DLS) was under 120 nm and polydispersity of the dual drug particles was around 0.2 (FIG. 29). Thus, these results further demonstrated that ratiometric loading of distinct types of drugs in DOPA coated cores could be achieved over a wide dual drug ratio range and loading efficiency.

### Example 7

### Characterization of Dual-Drug Loaded Combo NP Using TEM and XPS

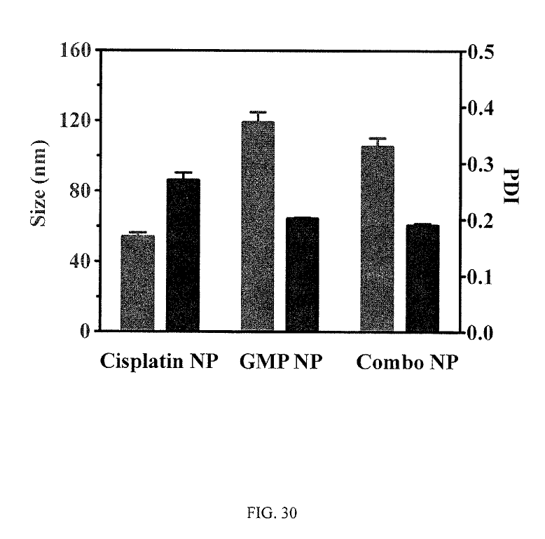

To demonstrate that GMP cores and CP cores are homogenously distributed in each Combo NP, tests further characterized the Combo NP with total drug feeding ratio of 6 wt % and feed GMP/cisplatin ratio of 5, whose determined loading was 5.5±0.8 wt % (n=5) and molar ratio between GMP and cisplatin was 5.3. TEM revealed Combo NP as spherical and mono-dispersed with a diameter of approximately 90-120 nm (FIG. 22C), which is consistent with the value measured by DLS (average 120 nm) (FIG. 30). In addition, large quantities of well-dispersed cores were clearly clustered in each NP, further confirming the hypothesis of a nanocapsule-like structure with high and efficient drug loading (FIG. 22C). Notably, each NP contained a similar amount of cores. However, TEM result alone cannot show the homogeneous distribution of cores in NP. Therefore, we further characterized the Combo NP using high resolution TEM with energy dispersive spectroscopy (EDS) analysis and x-ray photoelectron spectroscopy (XPS). Chemical element analysis using EDS indicated that both fluorine (characteristic element of GMP) and platinum (characteristic element of cisplatin) were present in single NP (FIG. 22D). Over 20 particles were analyzed to determine the average molar ratio of GMP and cisplatin inside each NP. The ratio of fluorine to platinum, representing the ratio of GMP to cisplatin, was approximately 4.9±1.9, which is comparable to the feed ratio of 5 and the determined ratio in bulk solution of 5.3. This result demonstrated that the two distinct cores were present in single NP and their ratio was precisely controlled. To avoid disturbance of neighboring oxygen on fluorine quantification, XPS was carried out to further confirm the ratiometric distribution of the two drugs. Combo NP was dissolved in THF, and a 5 nm layer of particle lysates were analyzed by XPS. The spectrum in FIG. 22E indicates that fluorine could be separated well from oxygen, and the calculated molar ratio of GMP to cisplatin was approximately 5.6, similar to the results determined using other techniques. Therefore, quantifications from the single particle nano-layer of particle lysate as well as the bulk solution strongly suggest the fact that the dual-drug combination has been successfully, homogenously loaded into single Combo NP with relatively precise ratiometric control.

### Example 8

### In Vitro Ratiometric Control Over Dual-Drug Cellular Uptake

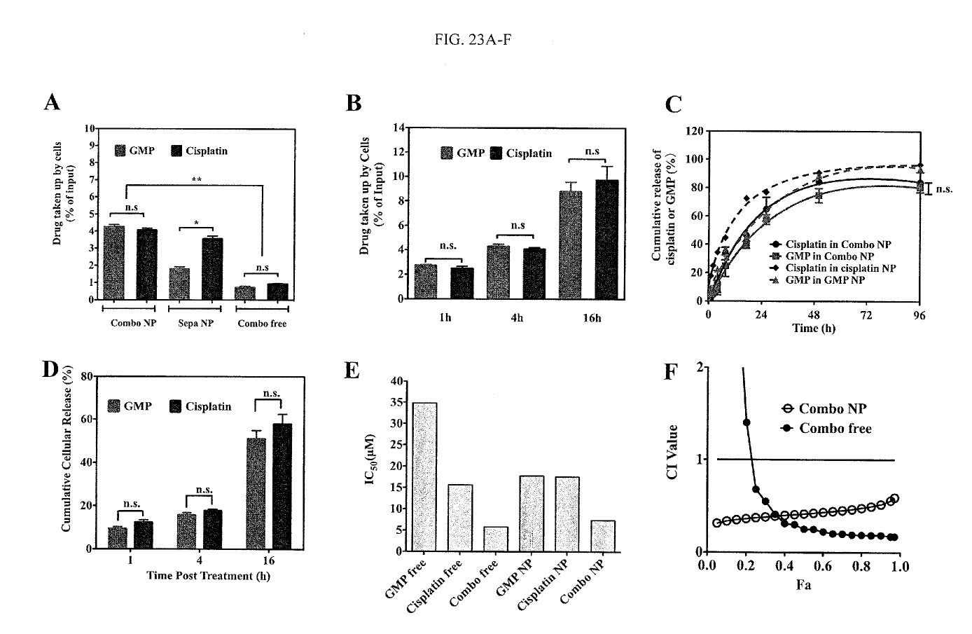

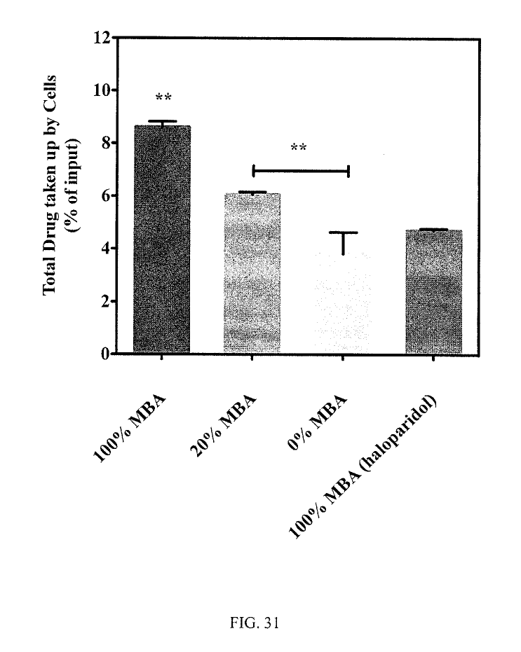

In vitro synergy studies of free cisplatin and GMP (Combo free) using Chou-Talalay method (T. C. Chou, P. Talalay, Adv. Enzyme Regul. 1984, 22, 27) indicated that Combo free exhibited the strongest synergy at a GMP/cisplatin ratio of 5 in human urinary bladder carcinoma UMUC3 cell line. (J. Zhang, L. Miao, S. Guo, Y. Zhang, L. Zhang, A. Satterlee, W. Y. Kim, L. Huang, J. Control. Release, DOI 10.1016/j.jconre1.2014.03.016). Herein, incorporation of ³H-labeled CMP (cytidine monophosphate) into single GMP cores in PLGA NP (GMP NP) was used a marker to detect the concentration of GMP. In vitro cellular uptake (FIG. 31) of free GMP and free cisplatin indicated that UMUC3 cells exhibited an equivalent uptake of GMP and cisplatin, suggesting that the feed ratio and the actual intracellular ratio of the drug combination was almost identical in the in vitro assay (FIG. 23A). However, the uptake of drugs in the tumor cells in vivo may be much different due to differing PK profiles and the complicated tumor microenvironment. In order to maintain the ratio of drugs in vivo and reveal the desired synergy of Combo free at a GMP/cisplatin ratio of 5, PLGA NP with a total drug loading of 5.5±0.8 wt % (n=5) and molar ratio between GMP and cisplatin of 5.3 were further investigated in the following studies. Single drug PLGA NP with a feed ratio of 6 wt % was used for comparison (Table 1). Notably, the size of CP cores in single PLGA NP (cisplatin NP) was smaller (approximately 60 nm) than that of GMP NP and Combo NP. CP cores are denser than GMP cores which are mainly composed of calcium phosphate. Ratiometric cellular uptake of both GMP and cisplatin by UMUC3 cells is a proposed prerequisite to evaluating synergistic effects. Cellular uptake of GMP and cisplatin in separate NP was compared with that of the dual-drug combination in Combo NP (FIG. 23A). Results indicated that Combo NP ratiometrically transported drugs into cells, which is consistent with the results from Combo free, while a mixture of separate NP (Sepa NP) cannot maintain the predetermined ratio of drugs because smaller cisplatin NP deliver their cargo into cells more efficiently than the larger GMP NP. This ratiometric uptake of Combo NP was also observed over a longer incubation of NP with cells (FIG. 23B).

TABLE 1
Characteristic features of the optimized single
drug PLGA NP and dual Drug PLGA Combo NP

| | CDDP PLGA | GMP PLGA | GMP & CDDP PLGA |
|---|---|---|---|
| Optimal | NP | NP | Combo NP |
| DL (wt %) | 4.4 ± 0.6 | 4.2 ± 0.3 | 5.5 ± 0.8 |
| EE (%) | 74.0 ± 10.0 | 69.5 ± 1.6 | 86.6 ± 1.9 & 92.4 ± 1.6 |

(n = 3)

### Example 9

### In Vitro Ratiometric Control Over Dual-Drug Release from PLGA NP

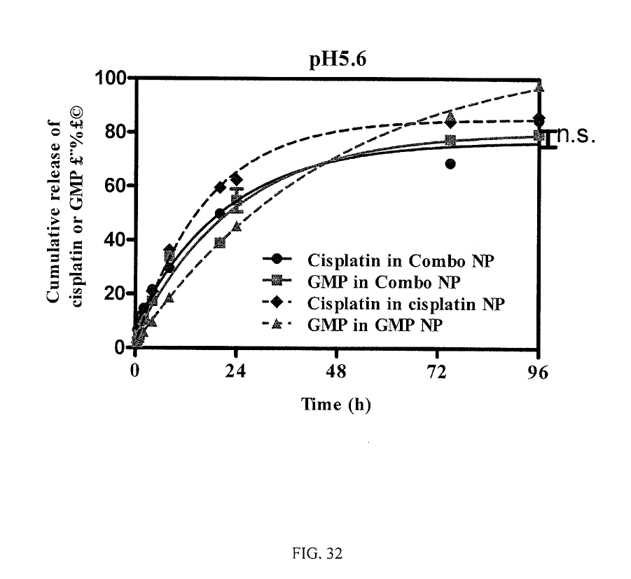

After verifying that Combo NP can ratiometrically transport the drugs into cells, studies were performed to determine the extracellular and intracellular release of Combo NP. The in vitro release kinetics of cisplatin and GMP from Combo NP, cisplatin NP and GMP NP were first investigated via dialysis in PBS (pH=7.4) at 37° C. for 96 h. The amount of platinum released from NP was measured by ICP-MS, while ³H-labeled CMP served as a marker for the measurement of GMP. It is notable that only negligible burst release was observed when the drugs inside DOPA-coated cores were encapsulated in PLGA NP (FIG. 23C), although burst release phenomenon is well known and commonly observed for hydrophilic drugs in PLGA nanoparticulate formulation. (a) K. Avgoustakis, A. Beletsi, Z. Panagi, P. Klepetsanis, A. G. Karydas, D. S. Ithakissios, J. Control. Release 2002, 79, 123; b) S. Aggarwal, S. Yadav, S. Gupta, J. Biomed. Nanotechnol. 2011, 7, 137; P. Pantazis, K. Dimas, J. H. Wyche, S. Anant, C. W. Houchen, J. Panyam, R P Ramanujam, Methods Mol. Biol. 2012, 906, 311). For example, cisplatin incorporated PLGA15K-PEG₅₀₀₀ NP have shown a burst release in the initial 4 h with a release fraction of approximately 50% and gemcitabine encapsulated PLGA NP have shown 60% liberated drug in the initial 6 h (Avgoustakis, et al., 2002; L. Martin-Banderas, E. Saez-Fernandez, M. A. Holgado, M. M. Duran-Lobato, J. C. Prados, C. Melguizo, J. L. Arias, Int. J. Pharm. 2013, 443, 103). This suggests that the DOPA layer prevents burst release of GMP and cisplatin from PLGA NP. Release kinetics of these two drugs in combination was further analyzed by grouped t-tests, which showed that there was no significant difference between these two drugs (p=0.78). This observation suggests that dual drugs in Combo NP followed a ratiometric release profile. The subtle difference in release rate may be due to the different composition of CP cores and GMP cores, yet the difference can be neglected when compared to the release rate of drugs from PLGA NP, which is a key rate-limiting step of the procedure. This indicates that release of cisplatin and GMP can be controlled at a similar rate and in a ratiometric manner when co-encapsulated into single PLGA NP. In order to further mimic the acidic endosome microenvironment (Z. Tong, W. Luo, Y. Wang, F. Yang, Y. Han, H. Li, H. Luo, B. Duan, T. Xu, Q. Maoying, H. Tan, J. Wang, H. Zhao, F. Liu, Y. Wan, PloS one 2010, 5, e10234), a release kinetics study was also carried out in pH 5.6 PBS for 96 h. There were subtle changes in the release kinetics of the drugs and the ratio-controlled release of the dual drugs in Combo NP was still well-maintained (FIG. 32).

Intracellular release of drugs from Combo NP was then studied. UMUC3 Cells were first incubated with Combo NP for 1, 4, or 16 h and subsequently washed. At each time points, cells were lysed with RIPA buffer, followed by separation of NP and free drugs via centrifugation at 16,000 g for 20 min. This method can extract more than 98% of NP and free drugs from cells with little destruction of NP. Results in FIG. 23D indicated that a controlled and ratiometric release of cisplatin and GMP were also observed in the UMUC3 at the cellular level.

### Example 10

### In Vitro Synergistic Effect of Combo NP

The in vitro cytotoxicity of free drugs and drug-loaded PLGA NP were evaluated by using the 3-(4,5-dimethylthiazol-2-yl)-2,5-diphenyltetrazolium bromide (MTT) assay. Results showed that although subtle differences between the half-maximal inhibitory concentration (IC₅₀) of free cisplatin and cisplatin NP existed, GMP NP resulted in a much lower IC₅₀ of 17.8 μM compared with GMP free drug (IC₅₀ of 34.8 μM), indicating that targeted NP delivery can maintain or enhance the cytotoxicity in vitro (FIG. 23E). In addition, data revealed blank PLGA NP containing CaP core with negligible toxicity (data not shown). To validate the in vitro synergistic effect of Combo NP with dual-drug molar ratio of 5.3:1 (GMP:cisplatin), the combination index (CI) was further determined using the isobologram equation of Chou-Talalay. (T. C. Chou, P. Talalay, Adv. Enzyme Regal. 1984, 22, 27). As shown in FIG. 23F, Combo NP displayed an overall CI value <1 when Fa value was in the validated range of 0.2 to 0.8, indicating the pronounced and clear synergy of PLGA combo therapy in vitro.

### Example 11

### In Vivo Anti-Cancer Efficacy of Combo NP on Stroma-Riched Bladder Xenograft Tumor Model

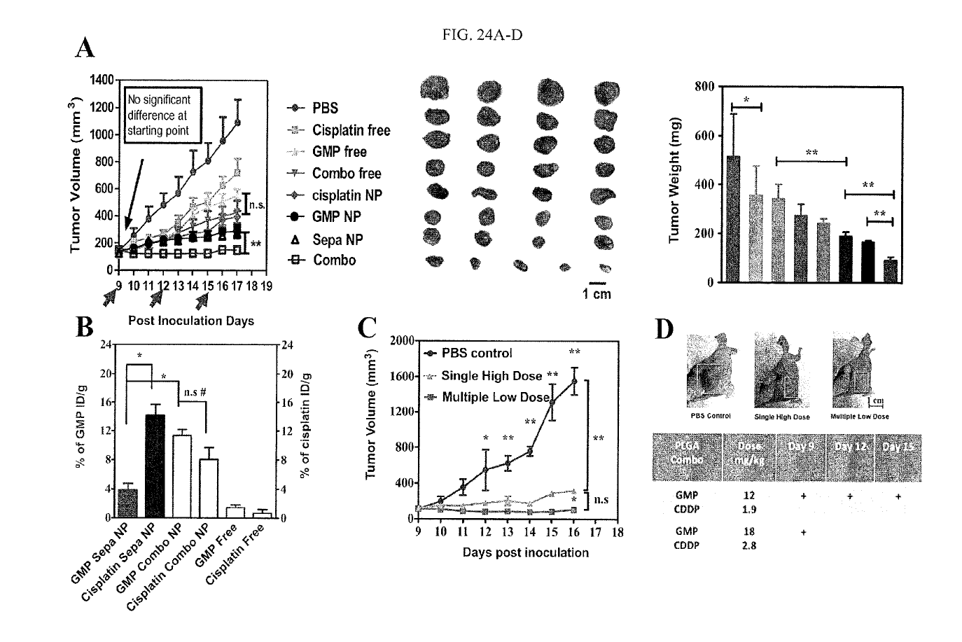

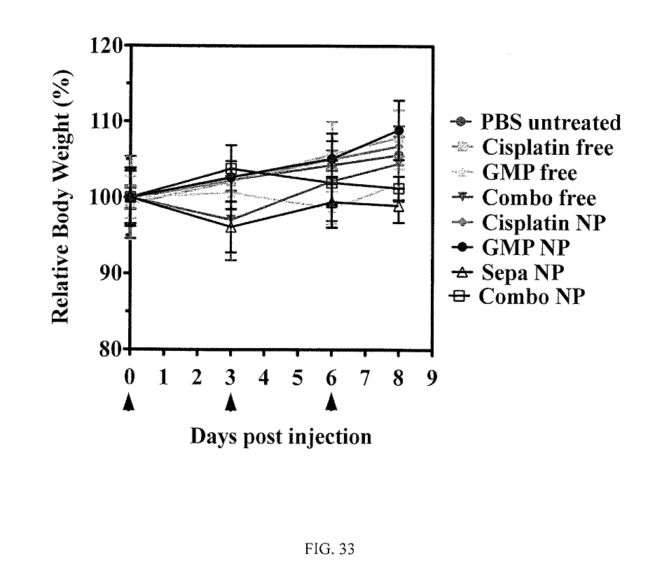

As previously mentioned, one of the most fundamental principles behind this formulation is to controllably deliver dual drugs into the tumor with an optimized ratio so as to achieve an enhanced anti-tumor efficacy in vivo. Therefore, different treatments were evaluated in an aggressive stroma-rich bladder cancer model, which was established by subcutaneously co-inoculating UMUC3 cells along with fibroblast NIH 3T3 cells in matrigel. Tumors were allowed to develop until their volume reached 100˜150 mm³. Tumor bearing mice were then treated with a total of 3 injections at a dose of 12 mg/kg GMP and 1.9 mg/kg cisplatin in Combo NP. Cisplatin and GMP prepared in separate PLGA NP (Sepa NP) were administrated simultaneously in a mixture for comparison. Blank PLGA NP have no tumor inhibition effect. (S. Guo, C. M. Lin, Z. Xu, L. Miao, Y. Wang, L. Huang, ACS Nano., DOI: 10.1021/nn5010815). As shown in FIG. 24A, free drugs showed little inhibitory effect at the same dose and dose schedule, possibly due to low tumor accumulation; while single drugs in PLGA NP demonstrated an enhanced therapeutic efficacy compared with free drugs. This is due to the EPR effect and receptor mediated endocytosis mentioned earlier. Dual drugs in Combo NP inhibited the growth of UMUC3 tumors most significantly without reducing the body weight (FIG. 24A and FIG. 33), indicating the enhanced anti-cancer effect and the safety of cisplatin and GMP in combination compared to single drugs. However, when the dual drugs were dosed together in a mixture (i.e., Sepa NP), tumor inhibition seemed to be compromised and the tumor weight on the last day of measurement was significantly higher than that of the Combo NP (FIG. 24A). To further confirm the potent anti-cancer efficacy of Combo NP in the aggressive UMUC3 tumor model, a single injection of high dose Combo NP was administered and compared with low dose at regular dosing intervals. Results indicated that GMP and cisplatin in single high dose Combo NP showed potent efficacy, which is comparable to the effect of low dose at regular dosing intervals. Thus, only single injection could inhibit tumor growth in the aggressive stroma-rich tumor model (FIG. 24C).

### Example 12

### In Vivo Ratiometric Control Over Dual-Drug Tumor Accumulation in Xenograft Tumor Model

Combo NP were more efficient in inhibiting growth of the tumor than Sepa NP potentially due to the fact that Combo NP may deliver cisplatin and GMP into the tumor at the predetermined optimized synergistic ratio and dose. Tumor accumulation data indicated ratiometric accumulation of GMP and cisplatin from Combo NP over 10 h post injection (FIG. 24B). However, higher uptake of cisplatin NP and lower uptake of GMP NP was observed after dosing with Sepa NP. On one hand, smaller particle size (around 60 nm) can account for higher tumor accumulation of cisplatin in single PLGA NP, while on the other hand, compared with 5.5 wt % loading of dual drugs in single PLGA NP, the same dose of 4.4 wt % cisplatin and 4.2 wt % GMP in separate PLGA NP doubles the amount of injected anisamide modified PLGA NP, which can result in saturation of sigma receptors and subsequently reduce the accumulation of GMP in tumors. This observation suggests advantages in controlling the ratio of drugs in DOPA coated cores in single PLGA NP over a mixture of separate NPs, which have variant physicochemical properties and distinct pharmacokinetics. Variations in the loaded ratio and actual amount of drug taken up by tumor tissues can directly affect the anti-tumor efficacy induced by synergy. In addition, nanoparticles also increased the tumor accumulation of free drugs from 2% ID/g to more than 10% ID/g due to the EPR effect and enhanced internalization into tumor cells through a receptor mediated pathway.

### Example 13

### Combo NP Triggered Significant Tumor Cell Apoptosis and Inhibited Tumor Cell Proliferation Effectively In Vivo in Stroma-Rich UMUC3 Xenografts

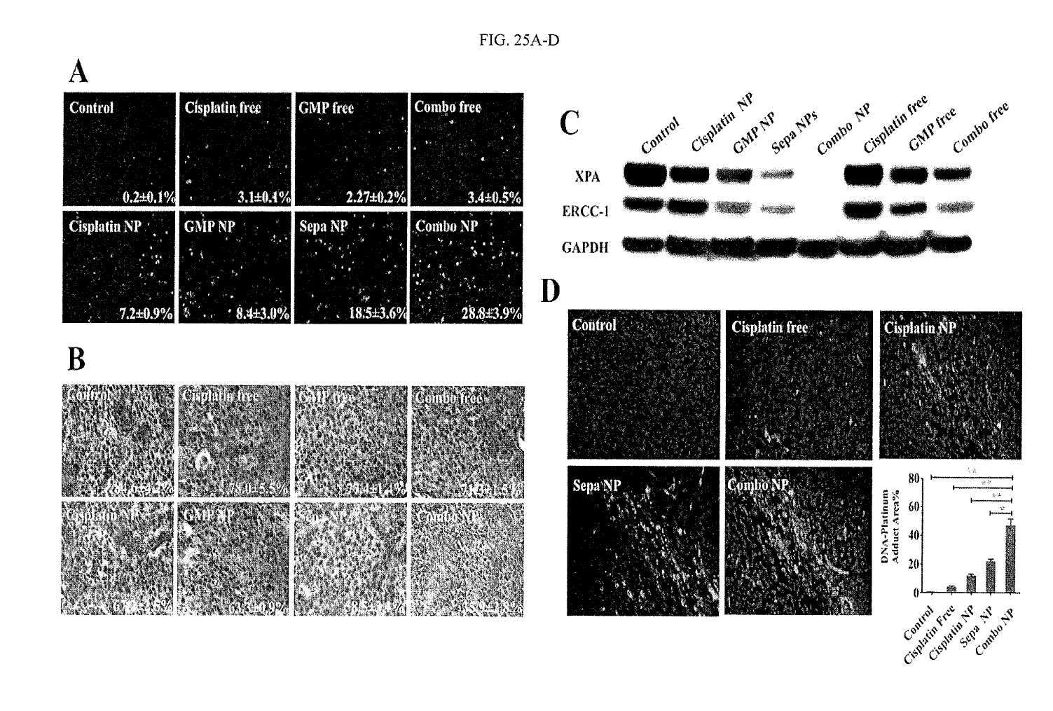

Enhanced antitumor efficacy of Combo NP was confirmed via analysis of apoptosis and proliferation. Tumor tissues after treatment were further sectioned for TUNEL (terminal deoxynucleotidyl transferase dUTP nick end labeling) assay and PCNA (proliferating cell nuclear antigen) immunohistochemistry (FIG. 25A and FIG. 25B). Results indicated that Combo NP induced apoptosis in 28.8% of cells in UMUC3 xenograft tumors. Dual drugs in Sepa NP caused more cell apoptosis compared with cisplatin NP and GMP NP treatment, but were still significantly less efficient in inducing apoptosis than Combo NP. Free drugs induced few apoptotic cells in vivo, probably because the majority of the free drugs were metabolized and cleared before they accumulated in the tumor. In addition, the inhibition of tumor cell proliferation was investigated using PCNA assay. PCNA is expressed in the cell nuclei during DNA synthesis and can be used as a marker for cell proliferation. PCNA results were consistent with those of TUNEL assay. Combo NP showed minimal amounts of PCNA positive cells. These data further illustrated that combined drugs in a single NP inhibited the growth of the tumor through enhanced induction of apoptosis and reduced cell proliferation.

### Example 14

### Mechanism of Synergistic Effect of the Dual-Drug Combo NP

In order to validate the observed enhanced antitumor effect of Combo NP is a synergistic effect imposed by GMP and cisplatin in the NP, subsequent studies were designed accordingly from a mechanistic basis. It is reported that gemcitabine potentiates the accumulation of cisplatin damage by suppressing the expression of key proteins involved in nucleotide excision repair (NER) and mismatch repair (MMR), leading to a decreased repair of Pt-DNA adducts, and thereby suppressed repair of cisplatin-induced DNA lesions. (O. G. Besancon, G. A. Tytgat, R. Meinsma, R. Leen, J. Hoebink, G. V. Kalayda, U. Jaehde, H. N. Caron, A. B. van Kuilenburg, Cancer Lett. 2012, 319, 23; a) H. P. C J A van Moorsel, G Veerman, A M Bergman, C M Kuiper, J B Vermorken, W J F van der Vijgh, Br. J. Cancer 1999, 80, 10; b) B. Liedert, D. Pluim, J. Schellens, J. Thomale, Nucleic Acids Res. 2006, 34, e47). Therefore, intensified inhibition of DNA repair and Pt-DNA adduct removal are two signs of synergistic interaction. The effect of combination therapy on ERCC1 and XPA (Beasancon et al., 2012), two major proteins with key roles in NER was first examined by western blotting and showed that down-regulation of ERCC1 and XPA was induced by GMP free drug and enhanced by GMP NP treatment (FIG. 25C). Combo NP almost completely depleted the expression of ERCC1 and XPA and was more efficient than Sepa NP. To study the effect of down-regulation of ERCC1 and XPA on Pt-DNA repair, Pt-DNA adducts were stained with FITC-labeled anti Pt-DNA adduct antibody. As shown in FIG. 25D, a significant increase in the amount of Pt-DNA adducts was observed when tumors were treated with Combo NP, compared with that of Sepa NP.

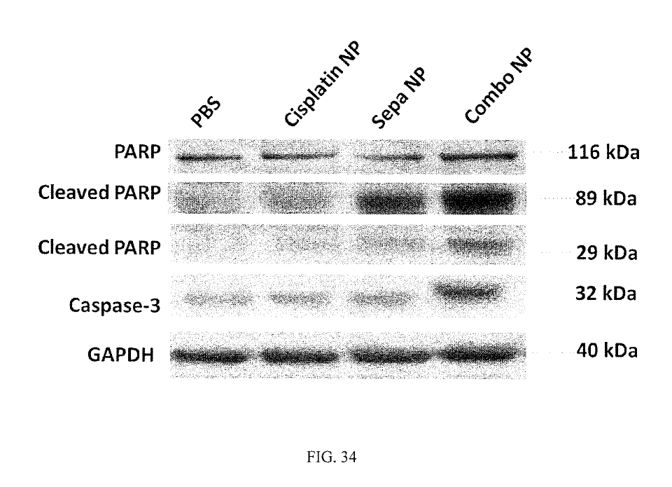

The level of cleaved PARP and Caspase-3 were observed in order to further investigate the relationship of the suppressed DNA repair proteins and apoptosis. During the execution phase of apoptosis, intact PARP is mainly cleaved by caspase-3 or caspase-7 to a larger fragment and a smaller fragment. Therefore, PARP cleavage serves as a reliable marker of apoptosis. (Y. Zhang, W. Y. Kim, L. Huang, Biomaterials 2013, 34, 3447; Y. Zhang, L. Peng, R. J. Mumper, L. Huang, Biomaterials 2013, 34, 8459). FIG. 34 indicated that cleaved PARP was significantly elevated after treatment with Combo NP, which is consistent with the results of the DNA repair proteins and Pt-DNA adduct formation. Caspase-3 was also elevated after Combo NP treatment. Conclusively, Combo NP exhibited greater efficacy in inhibiting DNA repair and suppressing the removal of Pt-DNA adducts, leading to intensified apoptosis compared to dual drugs in separate NP in vivo. These results further verify that Combo NP acted in a synergistic fashion rather than only additive fashion to induce the enhanced anti-cancer effect in the stroma-rich bladder cancer xenograft model.

### Example 15

### Evaluation of Systemic Toxicity of Combo NP

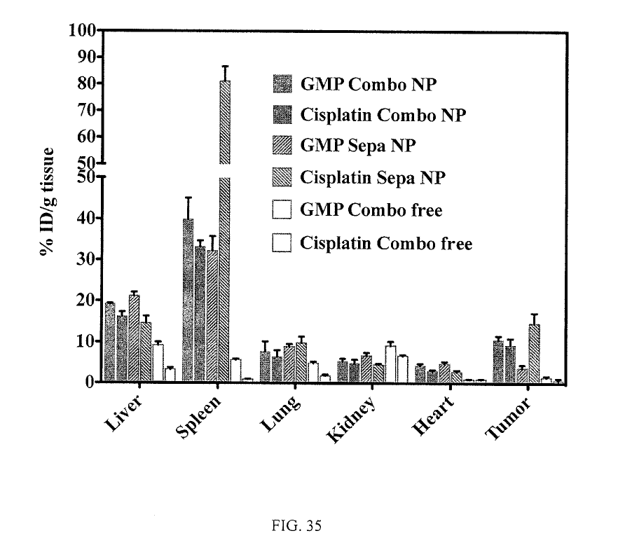

Another important issue involved with combination therapy is the dual-drug distribution and ratio in major organs, as well as, the association of synergistic effects with toxicity in these organs. Quantitative biodistribution analyses of GMP and cisplatin in Combo NP indicated that the ratio of dual drugs remained constant in almost all organs (FIG. 35) Similar to other nano-platforms, the major particle uptake organs were the liver (approximately 20% ID/g tissue) and the spleen (approximately 40% ID/g tissue) 10 h post injection. However, free drugs were eliminated rapidly from the body leaving the kidney as the major accumulation organ, which also explains the common nephrotoxicity of free cisplatin. Due to different particle size, cisplatin and GMP in separate nanoparticles presented very different distribution behaviors in vivo. Notably, cisplatin NP showed significantly higher accumulation in spleen, which might be a potential factor for inducing spleen toxicity.

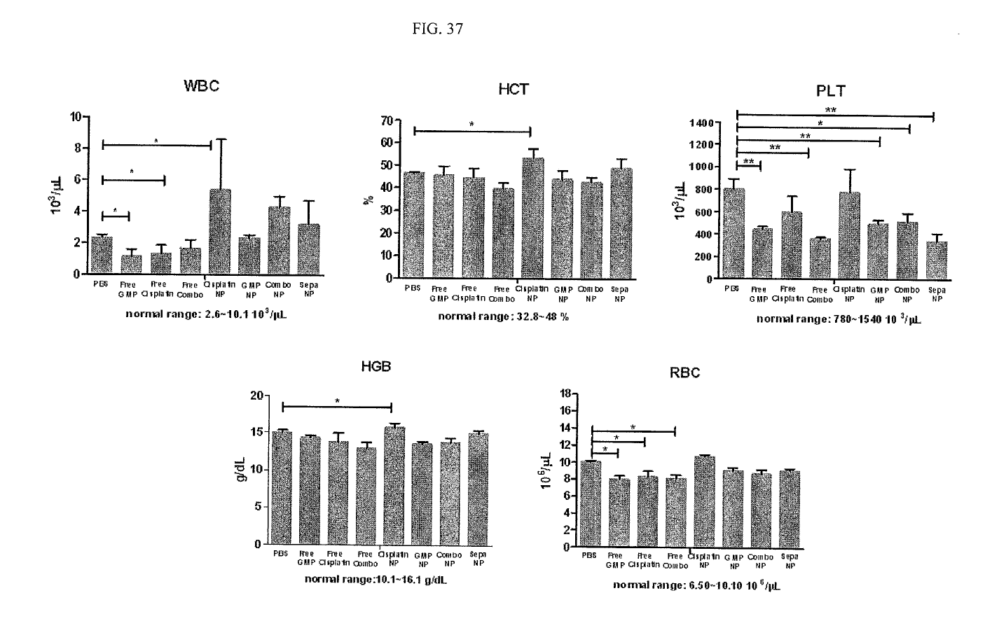

Since the major side effect of GMP is myelosuppression and cisplatin can also induce an accumulated decrease in hematopoietic cell counts, a blood routine test was performed on healthy nude mice with three dosages of the 8 treatment groups. Both free GMP and cisplatin significantly reduced the levels of red blood cells (RBC), platelets (PLT) and white blood cells (WBC) compared to untreated control (FIG. 37). Combination of these free drugs slightly potentiates the toxicity. Although there was an inevitable amount of accumulation of NP in the liver and kidney, blood biochemistry tests showed that NP coating can slightly alleviate the chemo-drug induced myelosuppression. There is no noticeable aggravation of blood toxicity in Combo NP. WBC, RBC, hematocrit (HCT), hemoglobin (HGB) of Combo NP were all close to the value of the untreated control (FIG. 37).

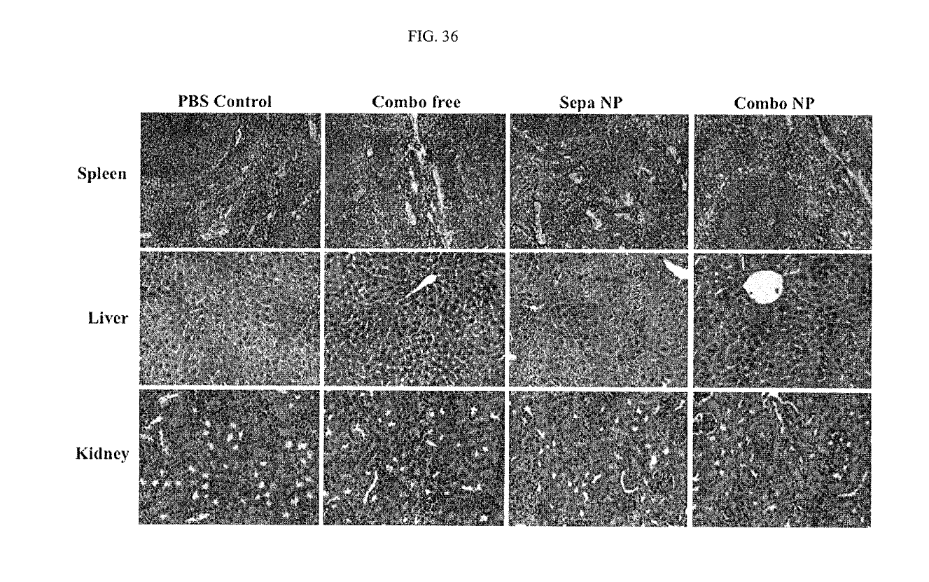

Other hematological parameters showed that no detectable damage was caused; aspartate aminotransferase (AST), alanine aminotransferase (ALT) and blood urea nitrogen (BUN) analyses were all within the normal range (Table 2). No noticeable histological changes were seen in H&E-stained tissue sections of the liver, kidney and spleen (FIG. 36). These studies demonstrated that Combo NP, with the most significant synergistic therapeutic efficacy, have elevated tumor uptake and low spleen accumulation, and as well exhibited no significant toxicity to major organs and tissues. Therefore, ratiometric synergistic combination therapy with non-overlapping toxicity is a promising strategy in overcoming drug resistance while enhancing anti-cancer effect.

TABLE 2
Effect of Different Treatments on serum
ALT, AST, BUN and creatinine levels

| Treatment | BUN mg/dL | Creatinine mg/dL | AST U/L | ALT U/L |
|---|---|---|---|---|
| PBS | 19 ± 1 | 0.2 | 228 ± 13 | 60 ± 14 |
| Cisplatin free | 25 ± 1 | 0.2 | 216 ± 15 | 59 ± 1 |
| GMP free | 24 ± 2 | 0.2 | 122 ± 20 | 47 ± 3 |
| Combo free | 22 ± 5 | 0.2 | 116 ± 18 | 60 ± 4 |
| Cisplatin NP | 28 ± 3 | 0.2 | 245 ± 22 | 58 ± 11 |
| GMP NP | 21 ± 1 | 0.2 | 86 ± 6 | 42 ± 2 |
| Sepa NP | 29 ± 2 | 0.3 | 238 ± 10 | 55 ± 8 |
| Combo NP | 18 ± 3 | 0.2 | 122 ± 12 | 52 ± 12 |
| Normal Range | 12-33 | 0.2-0.9 | 54-298 | 17-132 |

(n = 5)

Developing NP to simultaneously encapsulate drugs with different physicochemical properties with precise ratiometric loading and delivery is challenging but extremely desirable in the combination chemotherapy of malignant diseases. As disclosed herein, nanocapsule-like PLGA particles with payloads of GMP cores and cisplatin cores have been developed. These dual-drug loaded NP exhibited precise ratiometric control over drug loading, cellular uptake, in vitro release and in vivo tumor accumulation. Furthermore, this single NP with well-controlled optimal dual-drug ratio exhibited a more significant antitumor efficacy compared with dual drugs in a mixture of separate NPs. This provides a solution to the problems of formulating cisplatin and other groups of hydrophilic drugs for ratiometric combination therapy. Therefore, this single nanoparticulate delivery platform is an efficient and relatively safe candidate in particular for the treatment of human bladder cancer.

This nanomaterial-system with spatially separated modalities prevents functional interference between individual molecules. Also, this system provides a possible well controlled platform for co-delivery chemotherapy with other hydrophobic ligand coated inorganic NP (e.g. ion oxide NP, gold NP, quantum dots and upconversion NP) for photothermal and theranostic purposes.

It is to be noted that the term “a” or “an” entity refers to one or more of that entity; for example, “a nanoparticle” is understood to represent one or more nanoparticles. As such, the terms “a” (or “an”), “one or more,” and “at least one” can be used interchangeably herein.

Throughout this specification and the claims, the words “comprise,” “comprises,” and “comprising” are used in a non-exclusive sense, except where the context requires otherwise.

As used herein, the term “about,” when referring to a value is meant to encompass variations of, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the presently disclosed subject matter be limited to the specific values recited when defining a range.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the foregoing list of embodiments and appended claims. Although specific terms are employed herein, they are used in a generic and descriptive sense only and not for purposes of limitation.

That which is claimed:

1. A method of preparing a polymer nanoparticle comprising:
i. forming an organic phase by contacting a first lipid coated nano-precipitated core containing a first bioactive compound with, 1) a different hydrophobic bioactive compound in its free form and 2) a polymer; wherein said contacting is in an organic solvent that is miscible in water to form said organic phase; and
ii. contacting the organic phase with water to form said polymer nanoparticle comprising the first lipid coated core containing a first bioactive compound, and the hydrophobic bioactive compound.

2. The method of claim 1, wherein said first bioactive compound is a nano-precipitated salt.

3. The method of claim 1, wherein said first bioactive compound is a platinum coordination complex.

4. The method of claim 1, wherein said first bioactive compound is a cisplatin compound or derivative thereof.

5. The method of claim 1, wherein said first bioactive compound is a gemcitabine monophosphate-calcium phosphate.

6. The method of claim 1, wherein said hydrophobic bioactive compound is an anticancer compound.

7. The method of claim 1, wherein said hydrophobic bioactive compound is rapamycin.

8. The method of claim 1, wherein said first bioactive compound and said hydrophobic bioactive compound are present in ratiometric amounts.

9. The method of claim 1, wherein said first bioactive compound is cisplatin and said hydrophobic bioactive compound is rapamycin.

10. The method of claim 1, wherein said polymer is a biocompatible polymer.

11. The method of claim 10, wherein said polymer is PLGA.

12. The method of claim 1, wherein said organic solvent is miscible with water.

13. The method of claim 12, wherein said solvent is THF.

14. The method of claim 1, wherein said lipid is DOPA.

15. A method of preparing a polymer nanoparticle comprising:
i. forming an organic phase by contacting a first lipid coated nano-precipitated core containing a first bioactive compound with, 1) a second lipid coated nano-precipitated core containing a second bioactive compound that is different from the first bioactive compound and 2) a polymer; wherein said contacting is in an organic solvent that is miscible in water to form said organic phase; and
ii. contacting the organic phase with water to form said polymer nanoparticle comprising the lipid coated core containing a first bioactive compound, and the lipid coated core containing a second bioactive compound that is different from the first bioactive compound.

16. The method of claim 15, wherein said first bioactive compound is a nano-precipitated salt.

17. The method of claim 15, wherein said first bioactive compound is a platinum coordination complex.

18. The method of claim 15, wherein said first bioactive compound is a cisplatin compound or derivative thereof.

19. The method of claim 15, wherein said second bioactive compound is a gemcitabine monophosphate-calcium phosphate.

20. The method of claim 15, wherein said first bioactive compound and said hydrophobic bioactive compound are present in ratiometric amounts.

21. The method of claim 15, wherein said first bioactive compound is cisplatin and said second bioactive compound is gemcitabine monophosphate.

22. The method of claim 15, wherein said polymer is a biocompatible polymer.

23. The method of claim 22, wherein said polymer is PLGA.

24. The method of claim 15, wherein said organic solvent is miscible with water.

25. The method of claim 24, wherein said solvent is THF.

26. The method of claim 15, wherein said lipid is DOPA.

27. A PLGA nanoparticle comprising,
i. a lipid-coated nano-precipitate of a first bioactive compound; and
ii. a second bioactive compound that is different from said first bioactive compound, wherein the first and second bioactive compounds are present in controlled ratiometric proportions.

28. The nanoparticle of claim 27, wherein said first bioactive compound is CDDP.

29. The nanoparticle of claim 28, wherein said second bioactive compound is rapamycin.

30. The nanoparticle of claim 28, wherein said second bioactive compound is present as a lipid-coated nano-precipitate.

31. The nanoparticle of claim 30, wherein said second bioactive compound is gemcitabine phosphate.
